SEQUENCE LISTING (152 sequences)

SEQ ID NO: 1 - protein, 19 aa, Artificial Sequence
  Description of Artificial Sequence: Synthetic peptide
TFVYGGCRAKRNNFKSAED

SEQ ID NO: 2 - protein, 19 aa, Artificial Sequence
  Description of Artificial Sequence: Synthetic peptide
TFQYGGCMGNGNNFVTEKE

SEQ ID NO: 3 - protein, 19 aa, Artificial Sequence
  Description of Artificial Sequence: Synthetic peptide
PFFYGGCGGNRNNFDTEEY

SEQ ID NO: 4 - protein, 19 aa, Artificial Sequence
  Description of Artificial Sequence: Synthetic peptide
SFYYGGCLGNKNNYLREEE

SEQ ID NO: 5 - protein, 19 aa, Artificial Sequence
  Description of Artificial Sequence: Synthetic peptide
TFFYGGCRAKRNNFKRAKY

SEQ ID NO: 6 - protein, 19 aa, Artificial Sequence
  Description of Artificial Sequence: Synthetic peptide
TFFYGGCRGKRNNFKRAKY

SEQ ID NO: 7 - protein, 19 aa, Artificial Sequence
  Description of Artificial Sequence: Synthetic peptide
TFFYGGCRAKKNNYKRAKY

SEQ ID NO: 8 - protein, 19 aa, Artificial Sequence
  Description of Artificial Sequence: Synthetic peptide
TFFYGGCRGKKNNFKRAKY

SEQ ID NO: 9 - protein, 19 aa, Artificial Sequence
  Description of Artificial Sequence: Synthetic peptide
TFQYGGCRAKRNNFKRAKY

SEQ ID NO: 10 - protein, 19 aa, Artificial Sequence
  Description of Artificial Sequence: Synthetic peptide
TFQYGGCRGKKNNFKRAKY

SEQ ID NO: 11 - protein, 19 aa, Artificial Sequence
  Description of Artificial Sequence: Synthetic peptide
TFFYGGCLGKRNNFKRAKY

SEQ ID NO: 12 - protein, 19 aa, Artificial Sequence
  Description of Artificial Sequence: Synthetic peptide
TFFYGGSLGKRNNFKRAKY

SEQ ID NO: 13 - protein, 19 aa, Artificial Sequence
  Description of Artificial Sequence: Synthetic peptide
PFFYGGCGGKKNNFKRAKY

SEQ ID NO: 14 - protein, 19 aa, Artificial Sequence
  Description of Artificial Sequence: Synthetic peptide
TFFYGGCRGKGNNYKRAKY

SEQ ID NO: 15 - protein, 19 aa, Artificial Sequence
  Description of Artificial Sequence: Synthetic peptide
PFFYGGCRGKRNNFLRAKY

SEQ ID NO: 16 - protein, 19 aa, Artificial Sequence
  Description of Artificial Sequence: Synthetic peptide
TFFYGGCRGKRNNFKREKY

SEQ ID NO: 17 - protein, 19 aa, Artificial Sequence
  Description of Artificial Sequence: Synthetic peptide
PFFYGGCRAKKNNFKRAKE

SEQ ID NO: 18 - protein, 19 aa, Artificial Sequence
  Description of Artificial Sequence: Synthetic peptide
TFFYGGCRGKRNNFKRAKD

SEQ ID NO: 19 - protein, 19 aa, Artificial Sequence
  Description of Artificial Sequence: Synthetic peptide
TFFYGGCRAKRNNFDRAKY

SEQ ID NO: 20 - protein, 19 aa, Artificial Sequence
  Description of Artificial Sequence: Synthetic peptide
TFFYGGCRGKKNNFKRAEY

SEQ ID NO: 21 - protein, 19 aa, Artificial Sequence
  Description of Artificial Sequence: Synthetic peptide
PFFYGGCGANRNNFKRAKY

SEQ ID NO: 22 - protein, 19 aa, Artificial Sequence
  Description of Artificial Sequence: Synthetic peptide
TFFYGGCGGKKNNFKTAKY

SEQ ID NO: 23 - protein, 19 aa, Artificial Sequence
  Description of Artificial Sequence: Synthetic peptide
TFFYGGCRGNRNNFLRAKY

SEQ ID NO: 24 - protein, 19 aa, Artificial Sequence
  Description of Artificial Sequence: Synthetic peptide
TFFYGGCRGNRNNFKTAKY

SEQ ID NO: 25 - protein, 19 aa, Artificial Sequence
  Description of Artificial Sequence: Synthetic peptide
TFFYGGSRGNRNNFKTAKY

SEQ ID NO: 26 - protein, 19 aa, Artificial Sequence
  Description of Artificial Sequence: Synthetic peptide
TFFYGGCLGNGNNFKRAKY

SEQ ID NO: 27 - protein, 19 aa, Artificial Sequence
  Description of Artificial Sequence: Synthetic peptide
TFFYGGCLGNRNNFLRAKY

SEQ ID NO: 28 - protein, 19 aa, Artificial Sequence
  Description of Artificial Sequence: Synthetic peptide
TFFYGGCLGNRNNFKTAKY

SEQ ID NO: 29 - protein, 19 aa, Artificial Sequence
  Description of Artificial Sequence: Synthetic peptide
TFFYGGCRGNGNNFKSAKY

SEQ ID NO: 30 - protein, 19 aa, Artificial Sequence
  Description of Artificial Sequence: Synthetic peptide
TFFYGGCRGKKNNFDREKY

SEQ ID NO: 31 - protein, 19 aa, Artificial Sequence
  Description of Artificial Sequence: Synthetic peptide
TFFYGGCRGKRNNFLREKE

SEQ ID NO: 32 - protein, 19 aa, Artificial Sequence
  Description of Artificial Sequence: Synthetic peptide
TFFYGGCRGKGNNFDRAKY

SEQ ID NO: 33 - protein, 19 aa, Artificial Sequence
  Description of Artificial Sequence: Synthetic peptide
TFFYGGSRGKGNNFDRAKY

SEQ ID NO: 34 - protein, 19 aa, Artificial Sequence
  Description of Artificial Sequence: Synthetic peptide
TFFYGGCRGNGNNFVTAKY

SEQ ID NO: 35 - protein, 19 aa, Artificial Sequence
  Description of Artificial Sequence: Synthetic peptide
PFFYGGCGGKGNNYVTAKY

SEQ ID NO: 36 - protein, 19 aa, Artificial Sequence
  Description of Artificial Sequence: Synthetic peptide
TFFYGGCLGKGNNFLTAKY

SEQ ID NO: 37 - protein, 19 aa, Artificial Sequence
  Description of Artificial Sequence: Synthetic peptide
SFFYGGCLGNKNNFLTAKY

SEQ ID NO: 38 - protein, 19 aa, Artificial Sequence
  Description of Artificial Sequence: Synthetic peptide
TFFYGGCGGNKNNFVREKY

SEQ ID NO: 39 - protein, 19 aa, Artificial Sequence
  Description of Artificial Sequence: Synthetic peptide
TFFYGGCMGNKNNFVREKY

SEQ ID NO: 40 - protein, 19 aa, Artificial Sequence
  Description of Artificial Sequence: Synthetic peptide
TFFYGGSMGNKNNFVREKY

SEQ ID NO: 41 - protein, 19 aa, Artificial Sequence
  Description of Artificial Sequence: Synthetic peptide
PFFYGGCLGNRNNYVREKY

SEQ ID NO: 42 - protein, 19 aa, Artificial Sequence
  Description of Artificial Sequence: Synthetic peptide
TFFYGGCLGNRNNFVREKY

SEQ ID NO: 43 - protein, 19 aa, Artificial Sequence
  Description of Artificial Sequence: Synthetic peptide
TFFYGGCLGNKNNYVREKY

SEQ ID NO: 44 - protein, 19 aa, Artificial Sequence
  Description of Artificial Sequence: Synthetic peptide
TFFYGGCGGNGNNFLTAKY

SEQ ID NO: 45 - protein, 19 aa, Artificial Sequence
  Description of Artificial Sequence: Synthetic peptide
TFFYGGCRGNRNNFLTAEY

SEQ ID NO: 46 - protein, 19 aa, Artificial Sequence
  Description of Artificial Sequence: Synthetic peptide
TFFYGGCRGNGNNFKSAEY

SEQ ID NO: 47 - protein, 19 aa, Artificial Sequence
  Description of Artificial Sequence: Synthetic peptide
PFFYGGCLGNKNNFKTAEY

SEQ ID NO: 48 - protein, 19 aa, Artificial Sequence
  Description of Artificial Sequence: Synthetic peptide
TFFYGGCRGNRNNFKTEEY

SEQ ID NO: 49 - protein, 19 aa, Artificial Sequence
  Description of Artificial Sequence: Synthetic peptide
TFFYGGCRGKRNNFKTEED

SEQ ID NO: 50 - protein, 19 aa, Artificial Sequence
  Description of Artificial Sequence: Synthetic peptide
PFFYGGCGGNGNNFVREKY

SEQ ID NO: 51 - protein, 19 aa, Artificial Sequence
  Description of Artificial Sequence: Synthetic peptide
SFFYGGCMGNGNNFVREKY

SEQ ID NO: 52 - protein, 19 aa, Artificial Sequence
  Description of Artificial Sequence: Synthetic peptide
PFFYGGCGGNGNNFLREKY

SEQ ID NO: 53 - protein, 19 aa, Artificial Sequence
  Description of Artificial Sequence: Synthetic peptide
TFFYGGCLGNGNNFVREKY

SEQ ID NO: 54 - protein, 19 aa, Artificial Sequence
  Description of Artificial Sequence: Synthetic peptide
SFFYGGCLGNGNNYLREKY

SEQ ID NO: 55 - protein, 19 aa, Artificial Sequence
  Description of Artificial Sequence: Synthetic peptide
TFFYGGSLGNGNNFVREKY

SEQ ID NO: 56 - protein, 19 aa, Artificial Sequence
  Description of Artificial Sequence: Synthetic peptide
TFFYGGCRGNGNNFVTAEY

SEQ ID NO: 57 - protein, 19 aa, Artificial Sequence
  Description of Artificial Sequence: Synthetic peptide
TFFYGGCLGKGNNFVSAEY

SEQ ID NO: 58 - protein, 19 aa, Artificial Sequence
  Description of Artificial Sequence: Synthetic peptide
TFFYGGCLGNRNNFDRAEY

SEQ ID NO: 59 - protein, 19 aa, Artificial Sequence
  Description of Artificial Sequence: Synthetic peptide
TFFYGGCLGNRNNFLREEY

SEQ ID NO: 60 - protein, 19 aa, Artificial Sequence
  Description of Artificial Sequence: Synthetic peptide
TFFYGGCLGNKNNYLREEY

SEQ ID NO: 61 - protein, 19 aa, Artificial Sequence
  Description of Artificial Sequence: Synthetic peptide
PFFYGGCGGNRNNYLREEY

SEQ ID NO: 62 - protein, 19 aa, Artificial Sequence
  Description of Artificial Sequence: Synthetic peptide
PFFYGGSGGNRNNYLREEY

SEQ ID NO: 63 - protein, 19 aa, Artificial Sequence
  Description of Artificial Sequence: Synthetic peptide
MRPDFCLEPPYTGPCVARI

SEQ ID NO: 64 - protein, 21 aa, Artificial Sequence
  Description of Artificial Sequence: Synthetic peptide
ARIIRYFYNAKAGLCQTFVYG

SEQ ID NO: 65 - protein, 22 aa, Artificial Sequence
  Description of Artificial Sequence: Synthetic peptide
YGGCRAKRNNYKSAEDCMRTCG

SEQ ID NO: 66 - protein, 22 aa, Artificial Sequence
  Description of Artificial Sequence: Synthetic peptide
PDFCLEPPYTGPCVARIIRYFY

SEQ ID NO: 67 - protein, 19 aa, Artificial Sequence
  Description of Artificial Sequence: Synthetic peptide
TFFYGGCRGKRNNFKTEEY

SEQ ID NO: 68 - protein, 19 aa, Artificial Sequence
  Description of Artificial Sequence: Synthetic peptide
KFFYGGCRGKRNNFKTEEY

SEQ ID NO: 69 - protein, 19 aa, Artificial Sequence
  Description of Artificial Sequence: Synthetic peptide
TFYYGGCRGKRNNYKTEEY

SEQ ID NO: 70 - protein, 19 aa, Artificial Sequence
  Description of Artificial Sequence: Synthetic peptide
TFFYGGSRGKRNNFKTEEY

SEQ ID NO: 71 - protein, 20 aa, Artificial Sequence
  Description of Artificial Sequence: Synthetic peptide
CTFFYGCCRGKRNNFKTEEY

SEQ ID NO: 72 - protein, 20 aa, Artificial Sequence
  Description of Artificial Sequence: Synthetic peptide
TFFYGGCRGKRNNFKTEEYC

SEQ ID NO: 73 - protein, 20 aa, Artificial Sequence
  Description of Artificial Sequence: Synthetic peptide
CTFFYGSCRGKRNNFKTEEY

SEQ ID NO: 74 - protein, 20 aa, Artificial Sequence
  Description of Artificial Sequence: Synthetic peptide
TFFYGGSRGKRNNFKTEEYC

SEQ ID NO: 75 - protein, 19 aa, Artificial Sequence
  Description of Artificial Sequence: Synthetic peptide
PFFYGGCRGKRNNFKTEEY

SEQ ID NO: 76 - protein, 19 aa, Artificial Sequence
  Description of Artificial Sequence: Synthetic peptide
TFFYGGCRGKRNNFKTKEY

SEQ ID NO: 77 - protein, 19 aa, Artificial Sequence
  Description of Artificial Sequence: Synthetic peptide
TFFYGGKRGKRNNFKTEEY

SEQ ID NO: 78 - protein, 19 aa, Artificial Sequence
  Description of Artificial Sequence: Synthetic peptide
TFFYGGCRGKRNNFKTKRY

SEQ ID NO: 79 - protein, 19 aa, Artificial Sequence
  Description of Artificial Sequence: Synthetic peptide
TFFYGGKRGKRNNFKTAEY

SEQ ID NO: 80 - protein, 19 aa, Artificial Sequence
  Description of Artificial Sequence: Synthetic peptide
TFFYGGKRGKRNNFKTAGY

SEQ ID NO: 81 - protein, 19 aa, Artificial Sequence
  Description of Artificial Sequence: Synthetic peptide
TFFYGGKRGKRNNFKREKY

SEQ ID NO: 82 - protein, 19 aa, Artificial Sequence
  Description of Artificial Sequence: Synthetic peptide
TFFYGGKRGKRNNFKRAKY

SEQ ID NO: 83 - protein, 19 aa, Artificial Sequence
  Description of Artificial Sequence: Synthetic peptide
TFFYGGCLGNRNNFKTEEY

SEQ ID NO: 84 - protein, 19 aa, Artificial Sequence
  Description of Artificial Sequence: Synthetic peptide
TFFYGCGRGKRNNFKTEEY

SEQ ID NO: 85 - protein, 19 aa, Artificial Sequence
  Description of Artificial Sequence: Synthetic peptide
TFFYGGRCGKRNNFKTEEY

SEQ ID NO: 86 - protein, 19 aa, Artificial Sequence
  Description of Artificial Sequence: Synthetic peptide
TFFYGGCLGNGNNFDTEEE

SEQ ID NO: 87 - protein, 19 aa, Artificial Sequence
  Description of Artificial Sequence: Synthetic peptide
TFQYGGCRGKRNNFKTEEY

SEQ ID NO: 88 - protein, 19 aa, Artificial Sequence
  Description of Artificial Sequence: Synthetic peptide
YNKEFGTFNTKGCERGYRF

SEQ ID NO: 89 - protein, 19 aa, Artificial Sequence
  Description of Artificial Sequence: Synthetic peptide
RFKYGGCLGNMNNFETLEE

SEQ ID NO: 90 - protein, 19 aa, Artificial Sequence
  Description of Artificial Sequence: Synthetic peptide
RFKYGGCLGNKNNFLRLKY

SEQ ID NO: 91 - protein, 19 aa, Artificial Sequence
  Description of Artificial Sequence: Synthetic peptide
RFKYGGCLGNKNNYLRLKY

SEQ ID NO: 92 - protein, 22 aa, Artificial Sequence
  Description of Artificial Sequence: Synthetic peptide
KTKRKRKKQRVKIAYEEIFKNY

SEQ ID NO: 93 - protein, 15 aa, Artificial Sequence
  Description of Artificial Sequence: Synthetic peptide
KTKRKRKKQRVKIAY

SEQ ID NO: 94 - protein, 17 aa, Artificial Sequence
  Description of Artificial Sequence: Synthetic peptide
RGGRLSYSRRFSTSTGR

SEQ ID NO: 95 - protein, 10 aa, Artificial Sequence
  Description of Artificial Sequence: Synthetic peptide
RRLSYSRRRF

SEQ ID NO: 96 - protein, 16 aa, Artificial Sequence
  Description of Artificial Sequence: Synthetic peptide
RQIKIWFQNRRMKWKK

SEQ ID NO: 97 - protein, 19 aa, Artificial Sequence
  Description of Artificial Sequence: Synthetic peptide
TFFYGGSRGKRNNFKTEEY

SEQ ID NO: 98 - protein, 59 aa, Artificial Sequence
  Description of Artificial Sequence: Synthetic polypeptide
MRPDFCLEPPYTGPCVARIIRYFYNAKAGLCQTFVYGGCRAKRNNFKSAEDCMRTCGGA

SEQ ID NO: 99 - protein, 19 aa, Artificial Sequence
  Description of Artificial Sequence: Synthetic peptide
TFFYGGCRGKRNNFKTKEY

SEQ ID NO: 100 - protein, 19 aa, Artificial Sequence
  Description of Artificial Sequence: Synthetic peptide
RFKYGGCLGNKNNYLRLKY

SEQ ID NO: 101 - protein, 19 aa, Artificial Sequence
  Description of Artificial Sequence: Synthetic peptide
TFFYGGCRAKRNNFKRAKY

SEQ ID NO: 102 - protein, 35 aa, Artificial Sequence
  Description of Artificial Sequence: Synthetic polypeptide
NAKAGLCQTFVYGGCLAKRNNFESAEDCMRTCGGA

SEQ ID NO: 103 - protein, 24 aa, Artificial Sequence
  Description of Artificial Sequence: Synthetic peptide
YGGCRAKRNNFKSAEDCMRTCGGA

SEQ ID NO: 104 - protein, 22 aa, Artificial Sequence
  Description of Artificial Sequence: Synthetic peptide
GLCQTFVYGGCRAKRNNFKSAE

SEQ ID NO: 105 - protein, 20 aa, Artificial Sequence
  Description of Artificial Sequence: Synthetic peptide
LCQTFVYGGCEAKRNNFKSA

SEQ ID NO: 106 - skipped

SEQ ID NO: 107 - protein, 19 aa, Artificial Sequence
  Description of Artificial Sequence: Synthetic peptide
TFFYGGSRGKRNNFKTEEY

SEQ ID NO: 108 - protein, 19 aa, Artificial Sequence
  Description of Artificial Sequence: Synthetic peptide
RFFYGGSRGKRNNFKTEEY

SEQ ID NO: 109 - protein, 19 aa, Artificial Sequence
  Description of Artificial Sequence: Synthetic peptide
RFFYGGSRGKRNNFKTEEY

SEQ ID NO: 110 - protein, 19 aa, Artificial Sequence
  Description of Artificial Sequence: Synthetic peptide
RFFYGGSRGKRNNFRTEEY

SEQ ID NO: 111 - protein, 19 aa, Artificial Sequence
  Description of Artificial Sequence: Synthetic peptide
TFFYGGSRGKRNNFRTEEY

SEQ ID NO: 112 - protein, 19 aa, Artificial Sequence
  Description of Artificial Sequence: Synthetic peptide
TFFYGGSRGRRNNFRTEEY

SEQ ID NO: 113 - protein, 20 aa, Artificial Sequence
  Description of Artificial Sequence: Synthetic peptide
CTFFYGGSRGKRNNFKTEEY

SEQ ID NO: 114 - protein, 20 aa, Artificial Sequence
  Description of Artificial Sequence: Synthetic peptide
TFFYGGSRGKRNNFKTEEYC

SEQ ID NO: 115 - protein, 20 aa, Artificial Sequence
  Description of Artificial Sequence: Synthetic peptide
CTFFYGGSRGRRNNFRTEEY

SEQ ID NO: 116 - protein, 20 aa, Artificial Sequence
  Description of Artificial Sequence: Synthetic peptide
TFFYGGSRGRRNNFRTEEYC

SEQ ID NO: 117 - protein, 19 aa, Artificial Sequence
  Description of Artificial Sequence: Synthetic peptide
YEETKFNNRKGRSGGYFFT

SEQ ID NO: 118 - protein, 19 aa, Artificial Sequence
  Description of Artificial Sequence: Synthetic peptide
  MOD_RES (1)..(19): Xaa is Ala, Arg, Asn, Asp, Cys, Gln, Glu, Gly, His, Ile, Leu, Lys, Met, Phe, Pro, Ser, Thr, Trp, Tyr, and Val or absent
  MOD_RES (1)..(1): Xaa is Arg
  MOD_RES (7)..(7): Xaa is Ser or Cys
  MOD_RES (10)..(10): Xaa is Arg or Lys
  MOD_RES (15)..(15): Xaa is Arg or Lys
XXXXXXXXXXXXXXXXXXX

SEQ ID NO: 119 - protein, 6 aa, Artificial Sequence
  Description of Artificial Sequence: Synthetic peptide
  MOD_RES (3)..(3): Xaa is Asn or Gln
  MOD_RES (4)..(4): Xaa is Asn or Gln
  MOD_RES (5)..(5): Xaa is Phe, Tyr, or Trp
KRXXXK

SEQ ID NO: 120 - protein, 21 aa, Artificial Sequence
  Description of Artificial Sequence: Synthetic peptide
  MOD_RES (1)..(1): Xaa is Cys or absent
  MOD_RES (2)..(2): Xaa is Cys, Thr, or absent
  MOD_RES (3)..(3): Xaa is Cys, Phe, or absent
  MOD_RES (4)..(4): Xaa is Cys, Phe, or absent
  MOD_RES (5)..(5): Xaai s Cys, Tyr, or absent
  MOD_RES (6)..(6): Xaa is Cys, Gly, or absent
  MOD_RES (7)..(7): Xaa is Cys, Gly, or absent
  MOD_RES (8)..(8): Xaa is Cys, Ser, or absent
  MOD_RES (9)..(9): Xaa is Cys, Gly or absent
  MOD_RES (10)..(10): Xaa is Cys, Gly or absent
  MOD_RES (13)..(13): Xaa is Asn or Gln
  MOD_RES (14)..(14): Xaa is Asn or Gln
  MOD_RES (15)..(15): Xaa is Phe, Tyr, or Trp
  MOD_RES (17)..(17): Xaa is Cys, Tyr, Thr, or absent
  MOD_RES (18)..(18): Xaa is Cys, Tyr, Glu or absent
  MOD_RES (19)..(19): Xaa is Glu or absent
  MOD_RES (20)..(20): Xaa is Tyr or absent
  MOD_RES (21)..(21): Xaa is Cys or absent
XXXXXXXXXXKRXXXKXXXXX

SEQ ID NO: 121 - protein, 6 aa, Artificial Sequence
  Description of Artificial Sequence: Synthetic peptide
KRNNFK

SEQ ID NO: 122 - protein, 7 aa, Artificial Sequence
  Description of Artificial Sequence: Synthetic peptide
  MOD_RES (1)..(1): Lys or D-Lys
  MOD_RES (1)..(6): at least one of X1, X2, X5, or X6 is a D-amino acid
  MOD_RES (2)..(2): Arg or D-Arg
  MOD_RES (5)..(5): Phe or D-Phe
  MOD_RES (6)..(6): Lys or D-Lys
KRNNFKY

SEQ ID NO: 123 - protein, 8 aa, Artificial Sequence
  Description of Artificial Sequence: Synthetic peptide
  MOD_RES (1)..(1): Lys or D-Lys
  MOD_RES (1)..(7): at least one of X1, X2, X5, X6, or X7 is a D-amino acid
  MOD_RES (2)..(2): Arg or D-Arg
  MOD_RES (5)..(5): Phe or D-Phe
  MOD_RES (6)..(6): Lys or D-Lys
  MOD_RES (7)..(7): Tyr or D-Tyr
KRNNFKYC

SEQ ID NO: 124 - protein, 6 aa, Artificial Sequence
  Description of Artificial Sequence: Synthetic peptide
  MOD_RES (1)..(1): Xaa is Lys or D-Lys
  MOD_RES (2)..(2): Xaa is Arg or D-Arg
  MOD_RES (5)..(5): Xaa is Phe or D-Phe
  MOD_RES (6)..(6): Xaa is Lys or D-Lys
XXNNXX

SEQ ID NO: 125 - protein, 7 aa, Artificial Sequence
  Description of Artificial Sequence: Synthetic peptide
  MOD_RES (1)..(1): Xaa is Lys or D-Lys
  MOD_RES (2)..(2): Xaa is Arg or D-Arg
  MOD_RES (5)..(5): Xaa is Phe or D-Phe
  MOD_RES (6)..(6): Xaa is Lys or D-Lys
  MOD_RES (7)..(7): Xaa is Tyr or D-Tyr
XXNNXXX

SEQ ID NO: 126 - protein, 22 aa, Artificial Sequence
  Description of Artificial Sequence: Synthetic peptide
  MOD_RES (1)..(1): Xaa is Cys or absent
  MOD_RES (2)..(2): Xaa is Cys, Thr or absent
  MOD_RES (3)..(3): Xaa is Cys, Phe, or absent
  MOD_RES (4)..(4): Xaa is Cys, Phe, or absent
  MOD_RES (5)..(5): Xaa is Cys, Tyr, or absent
  MOD_RES (6)..(6): Xaa is Cys, Gly, or absent
  MOD_RES (7)..(7): Xaa is Cys, Gly, or absent
  MOD_RES (8)..(8): Xaa is Cys, Ser, or absent
  MOD_RES (9)..(9): Xaa is Cys, Arg, or absent
  MOD_RES (10)..(10): Xaa is Cys, Gly, or absent
  MOD_RES (11)..(11): Xaa is Lys or D-Lys
  MOD_RES (12)..(12): Xaa is Arg or D-Arg
  MOD_RES (15)..(15): Xaa is Phe or D-Phe
  MOD_RES (16)..(16): Xaa is Lys or D-Lys
  MOD_RES (17)..(17): Xaa is Tyr or D-Tyr
  MOD_RES (18)..(18): Xaa is Cys, Tyr, Thr, or absent
  MOD_RES (19)..(19): Xaa is Cys, Tyr, Glu, or absent
  MOD_RES (20)..(20): Xaa is Glu or absent
  MOD_RES (21)..(21): Xaa is Tyr or absent
  MOD_RES (22)..(22): Xaa is Cys or absent
XXXXXXXXXXXXNNXXXXXXXX

SEQ ID NO: 127 - protein, 19 aa, Artificial Sequence
  Description of Artificial Sequence: Synthetic peptide
  MOD_RES (8)..(8): Xaa is D-Arg
  MOD_RES (10)..(10): Xaa is D-Lys
  MOD_RES (11)..(11): Xaa is D-Arg
TFFYGGSXGXXNNFKTEEY

SEQ ID NO: 128 - protein, 18 aa, Artificial Sequence
  Description of Artificial Sequence: Synthetic peptide
FYGGSRGKRNNFKTEEYC

SEQ ID NO: 129 - protein, 18 aa, Artificial Sequence
  Description of Artificial Sequence: Synthetic peptide
  MOD_RES (8)..(8): Xaa is D-Lys
  MOD_RES (9)..(9): Xaa is D-Arg
  MOD_RES (12)..(12): Xaa is D-Phe
FYGGSRGXXNNXKTEEYC

SEQ ID NO: 130 - protein, 18 aa, Artificial Sequence
  Description of Artificial Sequence: Synthetic peptide
  MOD_RES (8)..(8): Xaa is D-Lys
  MOD_RES (9)..(9): Xaa is D-Arg
  MOD_RES (12)..(12): Xaa is D-Phe
  MOD_RES (13)..(13): Xaa is D-Lys
FYGGSRGXXNNXXTEEYC

SEQ ID NO: 131 - protein, 18 aa, Artificial Sequence
  Description of Artificial Sequence: Synthetic peptide
  MOD_RES (8)..(8): Xaa is D-Lys
  MOD_RES (9)..(9): Xaa is D-Arg
  MOD_RES (12)..(12): Xaa is D-Phe
  MOD_RES (13)..(13): Xaa is D-Lys
  MOD_RES (17)..(17): Xaa is D-Tyr
FYGGSRGXXNNXXTEEXC

SEQ ID NO: 132 - protein, 18 aa, Artificial Sequence
  Description of Artificial Sequence: Synthetic peptide
  MOD_RES (1)..(1): Xaa is D-Phe
  MOD_RES (2)..(2): Xaa is D-Tyr
  MOD_RES (6)..(6): Xaa is D-Arg
  MOD_RES (8)..(8): Xaa is D-Lys
  MOD_RES (9)..(9): Xaa is D-Arg
  MOD_RES (12)..(12): Xaa is D-Phe
  MOD_RES (13)..(13): Xaa is D-Lys
  MOD_RES (16)..(16): Xaa is D-Glu
  MOD_RES (17)..(17): Xaa is D-Tyr
XXGGSXGXXNNXXTEXXC

SEQ ID NO: 133 - protein, 16 aa, Artificial Sequence
  Description of Artificial Sequence: Synthetic peptide
GGSRGKRNNFKTEEYC

SEQ ID NO: 134 - protein, 14 aa, Artificial Sequence
  Description of Artificial Sequence: Synthetic peptide
SRGKRNNFKTEEYC

SEQ ID NO: 135 - protein, 12 aa, Artificial Sequence
  Description of Artificial Sequence: Synthetic peptide
GKRNNFKTEEYC

SEQ ID NO: 136 - protein, 11 aa, Artificial Sequence
  Description of Artificial Sequence: Synthetic peptide
KRNNFKTEEYC

SEQ ID NO: 137 - protein, 11 aa, Artificial Sequence
  Synthetic Construct
  MOD_RES (1)..(1): Xaa is D-Lys
  MOD_RES (2)..(2): Xaa is D-Arg
  MOD_RES (5)..(5): Xaa is D-Phe
XXNNXKTEEYC

SEQ ID NO: 138 - protein, 11 aa, Artificial Sequence
  Synthetic Construct
  MOD_RES (1)..(1): Xaa is D-Lys
  MOD_RES (2)..(2): Xaa is D-Arg
  MOD_RES (5)..(5): Xaa is D-Phe
  MOD_RES (6)..(6): Xaa is D-Lys
XXNNXXTEEYC

SEQ ID NO: 139 - protein, 11 aa, Artificial Sequence
  Synthetic Construct
  MOD_RES (1)..(1): Xaa is D-Lys
  MOD_RES (2)..(2): Xaa is D-Arg
  MOD_RES (5)..(5): Xaa is D-Phe
  MOD_RES (6)..(6): Xaa is D-Lys
  MOD_RES (10)..(10): Xaa is D-Tyr
XXNNXXTEEXC

SEQ ID NO: 140 - protein, 8 aa, Artificial Sequence
  Synthetic Construct
  MOD_RES (1)..(1): Xaa is D-Lys
  MOD_RES (2)..(2): Xaa is D-Arg
  MOD_RES (5)..(5): Xaa is D-Phe
XXNNXKYC

SEQ ID NO: 141 - protein, 9 aa, Artificial Sequence
  Synthetic Construct
  MOD_RES (1)..(1): Xaa is D-Lys
  MOD_RES (2)..(2): Xaa is D-Arg
  MOD_RES (5)..(5): Xaa is D-Phe
  MOD_RES (6)..(6): Xaa is D-Lys
XXNNXXKYC

SEQ ID NO: 142 - protein, 19 aa, Artificial Sequence
  Synthetic Construct
  MOD_RES (8)..(8): Xaa is D-Arg
  MOD_RES (10)..(10): Xaa is D-Lys
  MOD_RES (11)..(11): Xaa is D-Arg
  MOD_RES (15)..(15): Xaa is D-Lys
TFFYGGSXGXXNNFXTEEY

SEQ ID NO: 143 - protein, 8 aa, Artificial Sequence
  Synthetic Construct
  MOD_RES (1)..(1): Xaa is D-Lys
  MOD_RES (2)..(2): Xaa is D-Arg
  MOD_RES (5)..(5): Xaa is D-Phe
  MOD_RES (6)..(6): Xaa is D-Lys
  MOD_RES (7)..(7): Xaa is D-Tyr
XXNNXXXC

SEQ ID NO: 144 - protein, 16 aa, Artificial Sequence
  Synthetic Construct
FYGGSRGKRNNFKTEE

SEQ ID NO: 145 - protein, 14 aa, Artificial Sequence
  Synthetic Construct
GGSRGKRNNFKTEE

SEQ ID NO: 146 - protein, 12 aa, Artificial Sequence
  Synthetic Construct
SRGKRNNFKTEE

SEQ ID NO: 147 - protein, 10 aa, Artificial Sequence
  Synthetic Construct
GKRNNFKTEE

SEQ ID NO: 148 - protein, 9 aa, Artificial Sequence
  Synthetic Construct
KRNNFKTEE

SEQ ID NO: 149 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 150 - protein, 343 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGFLMRTNT

SEQ ID NO: 151 - protein, 460 aa, Homo sapiens
MPLRRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 152 - protein, 653 aa, Homo sapiens
MRPLRPRAALLALLASLLAAPPVAPAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

(19) United States
(12) Patent Application Publication
(10) Pub. No.: US 2015/0037311 A1
(43) Pub. Date: Feb. 5, 2015

(54) TARGETED LYSOSOMAL ENZYME COMPOUNDS

(71) Applicant: Angiochem Inc., Montreal (CA)

(72) Inventors: Dominique Boivin, Sainte-Marthe-Sur-Le-Lac (CA); Jean-Paul Castaigne, Mont-Royal (CA); Michel Demeule, Beaconsfield (CA); Sasmita Tripathy, Pierrefonds (CA); Jean-Christophe Currie, Repentigny (CA); Simon Lord-Dufour, Montreal (CA)

(73) Assignee: ANGIOCHEM INC., Montreal, QC (CA)

(21) Appl. No.: 14/362,034

(22) PCT Filed: Nov. 30, 2012
(86) PCT No.: PCT/CA2012/050867

§ 371 Date: May 30, 2014

Related U.S. Application Data
(60) Provisional application No. 61/660,564, filed on Jun. 15, 2012.
(60) Provisional application No. 61/596,515, filed on Feb. 8, 2012.
(60) Provisional application No. 61/565,764, filed on Dec. 1, 2011.

(51) Int. Cl. A61K 47/48; A61K 38/46

(52) U.S. Cl. CPC A61K 47/48246; A61K 38/465; C12Y 301/06013
USPC 424/94.6; 435/196; 435/188

(57) ABSTRACT

The present invention is related to a compound that includes a lysosomal enzyme and a targeting moiety, for example, where compound is a fusion protein including iduronate-2-sulfatase and Angiopep-2. In certain embodiments, these compounds, owning to the presence of the targeting moiety can crossing the blood-brain barrier or accumulate in the lysosome more effectively than the enzyme alone. The invention also features methods for treating lysosomal storage disorders (e.g., mucopolysaccharidosis Type II) using such compounds.

### BACKGROUND OF THE INVENTION

The invention relates to compounds including a lysosomal enzyme and a targeting moiety and the use of such conjugates in the treatment of disorders that result from a deficiency of such enzymes.

Lysosomal storage disorders are group of about 50 rare genetic disorders in which a subject has a defect in a lysosomal enzyme that is required for proper metabolism. These diseases typically result from autosomal or X-linked recessive genes. As a group, the incidence of these disorders is about 1:5000 to 1:10,000.

Hunter syndrome or mucopolysaccharidosis Type II (MPS-II) results from a deficiency of iduronate-2-sulfatase (IDS; also known as idursulfase), an enzyme that is required for lysosomal degradation of heparin sulfate and dermatan sulfate. Because the disorder is X-linked recessive, it primarily affects males. Those with the disorder are unable to break down and recycle these mucopolysaccharides, which are also known as glycosaminoglycans or GAG. This deficiency results in the buildup of GAG throughout the body, which has serious effects on the nervous system, joints, various organ systems including heart, liver, and skin. There are also a number of physical symptoms, including coarse facial features, enlarged head and abdomen, and skin lesions. In the most severe cases, the disease can be fatal in teen years and is accompanied by severe mental retardation.

There is no cure for MPS-II. In addition to palliative measures, therapeutic approaches have included bone marrow grafts and enzyme replacement therapy. Bone marrow grafts have been observed to stabilize the peripheral symptoms of MPS-II, including cardiovascular abnormalities, hepatosplenomegaly (enlarged liver and spleen), joint stiffness. This approach, however, did not stabilize or resolve the neuropsychological symptoms associated with this disease (Guffon et al., J. Pediatr. 154:733-7, 2009).

Enzyme replacement therapy by intravenous administration of IDS has also been shown to have benefits, including improvement in skin lesions (Marin et al., [published online ahead of print] Pediatr. Dermatol. Oct. 13, 2011), visceral organ size, gastrointestinal functioning, and reduced need for antibiotics to treat upper airway infections (Hoffman et al., Pediatr. Neurol. 45:181-4, 2011). Like bone marrow grafts, this approach does not improve the central nervous system deficits associated with MPS-II because the enzyme is not expected to cross the blood-brain barrier (BBB; Wraith et al., Eur. J. Pediatr. 1676:267-7, 2008).

Methods for increasing delivery of IDS to the brain have been and are being investigated, including intrathecal delivery (Felice et al., Toxicol. Pathol. 39:879-92, 2011). Intrathecal delivery, however, is a highly invasive technique.

Less invasive and more effective methods of treating MPS-II that address the neurological disease symptoms, in addition to the other symptoms, would therefore be highly desirable.

### SUMMARY OF THE INVENTION

The present invention is directed to compounds that include a targeting moiety and a lysosomal enzyme. These compounds are exemplified by IDS-Angiopep-2 conjugates and fusion proteins which can be used to treat MPS-II. Because these conjugates and fusion proteins are capable of crossing the BBB, they can treat not only the peripheral disease symptoms, but may also be effective in treating CNS symptoms. In addition, because targeting moieties such as Angiopep-2 are capable of targeting enzymes to the lysosomes, it is expected that these conjugates and fusion proteins are more effective than the enzymes by themselves.

Accordingly, in a first aspect, the invention features a compound including (a) a targeting moiety (e.g., a peptide or peptidic targeting moiety that may be less than 200, 150, 125, 100, 80, 60, 50, 40, 35, 30, 25, 24, 23, 22, 21, 20, or 19 amino acids) and (b) a lysosomal enzyme, an active fragment thereof, or an analog thereof, where the targeting moiety and the enzyme, fragment, or analog are joined by a linker. The lysosomal enzyme may be iduronate-2-sulfatase (IDS), an IDS fragment having IDS activity, or an IDS analog. In certain embodiments, the IDS enzyme or the IDS fragment has the amino acid sequence of human IDS isoform a or a fragment thereof (e.g., amino acids 26-550 of isoform a) or the IDS analog is substantially identical (e.g., at least 60%, 70%, 80%, 85%, 90%, 95%, 96%, 97%, 98%, or 99% identical) to the sequence of human IDS isoform a, isoform b, isoform c, or to amino acids 26-550 of isoform a. In a particular embodiment, the IDS enzyme has the sequence of human IDS isoform a or the mature form of isoform a (amino acids 26-550 of isoform a).

In the first aspect, the targeting moiety may include an amino acid sequence that is substantially identical to any of SEQ ID NOS:1-105 and 107-117 (e.g., Angiopep-2 (SEQ ID NO:97)). In other embodiments, the targeting moiety includes the formula Lys-Arg-X3-X4-X5-Lys (formula Ia), where X3 is Asn or Gln; X4 is Asn or Gln; and X5 is Phe, Tyr, or Trp, where the targeting moiety optionally includes one or more D-isomers of an amino acid recited in formula Ia. In other embodiments, the targeting moiety includes the formula Z1-Lys-Arg-X3-X4-X5-Lys-Z2 (formula Ib), where X3 is Asn or Gln; X4 is Asn or Gln; X5 is Phe, Tyr, or Trp; Z1 is absent, Cys, Gly, Cys-Gly, Arg-Gly, Cys-Arg-Gly, Ser-Arg-Gly, Cys-Ser-Arg-Gly, Gly-Ser-Arg-Gly, Cys-Gly-Ser-Arg-Gly, Gly-Gly-Ser-Arg-Gly, Cys-Gly-Gly-Ser-Arg-Gly, Tyr-Gly-Gly-Ser-Arg-Gly, Cys-Tyr-Gly-Gly-Ser-Arg-Gly, Phe-Tyr-Gly-Gly-Ser-Arg-Gly, Cys-Phe-Tyr-Gly-Gly-Ser-Arg-Gly, Phe-Phe-Tyr-Gly-Gly-Ser-Arg-Gly, Cys-Phe-Phe-Tyr-Gly-Gly-Ser-Arg-Gly, Thr-Phe-Phe-Tyr-Gly-Gly-Ser-Arg-Gly, or Cys-Thr-Phe-Phe-Tyr-Gly-Gly-Ser-Arg-Gly; and Z2 is absent, Cys, Tyr, Tyr-Cys, Cys-Tyr, Thr-Glu-Glu-Tyr, or Thr-Glu-Glu-Tyr-Cys; and where the targeting moiety optionally includes one or more D-isomers of an amino acid recited in formula Ib, Z1, or Z2. In other embodiments, the targeting moiety includes the formula X1-X2-Asn-Asn-X5-X6 (formula IIa), where X1 is Lys or D-Lys; X2 is Arg or D-Arg; X5 is Phe or D-Phe; and X6 is Lys or D-Lys; and where at least one of X1, X2, X5, or X6 is a D-amino acid. In other embodiments, the targeting moiety includes the formula X1-X2-Asn-Asn-X5-X6-X7 (formula IIb), where X1 is Lys or D-Lys; X2 is Arg or D-Arg; X5 is Phe or D-Phe; X6 is Lys or D-Lys; and X7 is Tyr or D-Tyr; and where at least one of X1, X2, X5, X6, or X7 is a D-amino acid. In other embodiments, the targeting moiety includes the formula Z1-X1-X2-Asn-Asn-X5-X6-X7-Z2 (formula IIc), where X1 is Lys or D-Lys; X2 is Arg or D-Arg; X5 is Phe or D-Phe; X6 is Lys or D-Lys; X7 is Tyr or D-Tyr; Z1 is absent, Cys, Gly, Cys-Gly, Arg-Gly, Cys-Arg-Gly, Ser-Arg-Gly, Cys-Ser-Arg-Gly, Gly-Ser-Arg-Gly, Cys-Gly-Ser-Arg-Gly, Gly-Gly-Ser-Arg-Gly, Cys-Gly-Gly-Ser-Arg-Gly, Tyr-Gly-Gly-Ser-Arg-Gly, Cys-Tyr-Gly-Gly-Ser-Arg-Gly, Phe-Tyr-Gly-Gly-Ser-Arg-Gly, Cys-Phe-Tyr-Gly-Gly-Ser-Arg-Gly, Phe-Phe-Tyr-Gly-Gly-Ser-Arg-Gly, Cys-Phe-Phe-Tyr-Gly-Gly-Ser-Arg-Gly, Thr-Phe-Phe-Tyr-Gly-Gly-Ser-Arg-Gly, or Cys-Thr-Phe-Phe-Tyr-Gly-Gly-Ser-Arg-Gly; and Z2 is absent, Cys, Tyr, Tyr-Cys, Cys-Tyr, Thr-Glu-Glu-Tyr, or Thr-Glu-Glu-Tyr-Cys; where at least one of X1, X2, X5, X6, or X7 is a D-amino acid; and where the polypeptide optionally includes one or more D-isomers of an amino acid recited in Z1 or Z2.

In the first aspect, the linker may be a covalent bond (e.g., a peptide bond) or one or more amino acids. The compound may be a fusion protein (e.g., Angiopep-2-IDS, IDS-Angiopep-2, or Angiopep-2-IDS-Angiopep-2, or has the structure shown in FIG. 1). The compound may further include a second targeting moiety that is joined to the compound by a second linker.

The invention also features a pharmaceutical composition including a compound of the first aspect and a pharmaceutically acceptable carrier.

In another aspect, the invention features a method of treating or treating prophylactically a subject having a lysosomal storage disorder (e.g., MPS-II). The method includes administering to the subject a compound of the first aspect or a pharmaceutical composition described herein. The lysosomal enzyme in the compound may be IDS. The subject may have either the severe form of MPS-II or the attenuated form of MPS-II. The subject may be experiencing neurological symptoms (e.g., mental retardation). The method may be performed on or started on a subject that is less than six months, or 1, 2, 3, 4, 5, 6, 7, 8, 9, 10, 11, 12, 13, 15, or 18 years of age. The subject may be an infant (e.g., less than 1 year old).

In certain embodiments, the targeting moiety is not an antibody (e.g., an antibody or an immunoglobulin that is specific for an endogenous BBB receptor such as the insulin receptor, the transferrin receptor, the leptin receptor, the lipoprotein receptor, and the IGF receptor).

In any of the above aspects, the targeting moiety may be substantially identical to any of the sequences of Table 1, or a fragment thereof. In certain embodiments, the peptide vector has a sequence of Angiopep-1 (SEQ ID NO:67), Angiopep-2 (SEQ ID NO:97) (An2), Angiopep-3 (SEQ ID NO:107), Angiopep-4a (SEQ ID NO:108), Angiopep-4b (SEQ ID NO:109), Angiopep-5 (SEQ ID NO:110), Angiopep-6 (SEQ ID NO:111), Angiopep-7 (SEQ ID NO:112)) or reversed Angiopep-2 (SEQ ID NO:117). The targeting moiety or compound may be efficiently transported into a particular cell type (e.g., any one, two, three, four, or five of liver, lung, kidney, spleen, and muscle) or may cross the mammalian BBB efficiently (e.g., Angiopep-1, -2, -3, -4a, -4b, -5, and -6). In another embodiment, the targeting moiety or compound is able to enter a particular cell type (e.g., any one, two, three, four, or five of liver, lung, kidney, spleen, and muscle) but does not cross the BBB efficiently (e.g., a conjugate including Angiopep-7). The targeting moiety may be of any length, for example, at least 6, 7, 8, 9, 10, 11, 12, 13, 14, 15, 16, 17, 18, 19, 20, 21, 25, 35, 50, 75, 100, 200, or 500 amino acids, or any range between these numbers. In certain embodiments, the targeting moiety is less than 200, 150, 125, 100, 90, 80, 70, 60, 50, 40, 30, 25, 24, 23, 22, 21, 20, 19, 18, 17, 16, 15, 14, 13, 12, 11, 10, 9, 8, 7, or 6 amino acids (e.g., 10 to 50 amino acids in length). The targeting moiety may be produced by recombinant genetic technology or chemical synthesis.

TABLE 1
Exemplary targeting moieties

| SEQ ID | | | | | | | | | | | | | | | | | | | | | | |
|---|---|---|---|---|---|---|---|---|---|---|---|---|---|---|---|---|---|---|---|---|---|---|
| NO: | | | | | | | | | | | | | | | | | | | | | | |
| 1 | T | F | V | Y | G | G | C | R | A | K | R | N | N | F | K | S | A | E | D | | | |
| 2 | T | F | Q | Y | G | G | C | M | G | N | G | N | N | F | V | T | E | K | E | | | |
| 3 | P | F | F | Y | G | G | C | G | G | N | R | N | N | F | D | T | E | E | Y | | | |
| 4 | S | F | Y | Y | G | G | C | L | G | N | K | N | N | Y | L | R | E | E | E | | | |
| 5 | T | F | F | Y | G | G | C | R | A | K | R | N | N | F | K | R | A | K | Y | | | |
| 6 | T | F | F | Y | G | G | C | R | G | K | R | N | N | F | K | R | A | K | Y | | | |
| 7 | T | F | F | Y | G | G | C | R | A | K | K | N | N | Y | K | R | A | K | Y | | | |
| 8 | T | F | F | Y | G | G | C | R | G | K | K | N | N | F | K | R | A | K | Y | | | |
| 9 | T | F | Q | Y | G | G | C | R | A | K | R | N | N | F | K | R | A | K | Y | | | |
| 10 | T | F | Q | Y | G | G | C | R | G | K | K | N | N | F | K | R | A | K | Y | | | |
| 11 | T | F | F | Y | G | G | C | L | G | K | R | N | N | F | K | R | A | K | Y | | | |
| 12 | T | F | F | Y | G | G | S | L | G | K | R | N | N | F | K | R | A | K | Y | | | |
| 13 | P | F | F | Y | G | G | C | G | G | K | K | N | N | F | K | R | A | K | Y | | | |
| 14 | T | F | F | Y | G | G | C | R | G | K | G | N | N | Y | K | R | A | K | Y | | | |
| 15 | P | F | F | Y | G | G | C | R | G | K | R | N | N | F | L | R | A | K | Y | | | |
| 16 | T | F | F | Y | G | G | C | R | G | K | R | N | N | F | K | R | E | K | Y | | | |
| 17 | P | F | F | Y | G | G | C | R | A | K | K | N | N | F | K | R | A | K | E | | | |
| 18 | T | F | F | Y | G | G | C | R | G | K | R | N | N | F | K | R | A | K | D | | | |
| 19 | T | F | F | Y | G | G | C | R | A | K | R | N | N | F | D | R | A | K | Y | | | |
| 20 | T | F | F | Y | G | G | C | R | G | K | K | N | N | F | K | R | A | E | Y | | | |
| 21 | P | F | F | Y | G | G | C | G | A | N | R | N | N | F | K | R | A | K | Y | | | |
| 22 | T | F | F | Y | G | G | C | G | G | K | K | N | N | F | K | T | A | K | Y | | | |
| 23 | T | F | F | Y | G | G | C | R | G | N | R | N | N | F | L | R | A | K | Y | | | |
| 24 | T | F | F | Y | G | G | C | R | G | N | R | N | N | F | K | T | A | K | Y | | | |
| 25 | T | F | F | Y | G | G | S | R | G | N | R | N | N | F | K | T | A | K | Y | | | |
| 26 | T | F | F | Y | G | G | C | L | G | N | G | N | N | F | K | R | A | K | Y | | | |
| 27 | T | F | F | Y | G | G | C | L | G | N | R | N | N | F | L | R | A | K | Y | | | |
| 28 | T | F | F | Y | G | G | C | L | G | N | R | N | N | F | K | T | A | K | Y | | | |
| 29 | T | F | F | Y | G | G | C | R | G | N | G | N | N | F | K | S | A | K | Y | | | |
| 30 | T | F | F | Y | G | G | C | R | G | K | K | N | N | F | D | R | E | K | Y | | | |
| 31 | T | F | F | Y | G | G | C | R | G | K | R | N | N | F | L | R | E | K | E | | | |
| 32 | T | F | F | Y | G | G | C | R | G | K | G | N | N | F | D | R | A | K | Y | | | |
| 33 | T | F | F | Y | G | G | S | R | G | K | G | N | N | F | D | R | A | K | Y | | | |
| 34 | T | F | F | Y | G | G | C | R | G | N | G | N | N | F | V | T | A | K | Y | | | |
| 35 | P | F | F | Y | G | G | C | G | G | K | G | N | N | Y | V | T | A | K | Y | | | |
| 36 | T | F | F | Y | G | G | C | L | G | K | G | N | N | F | L | T | A | K | Y | | | |
| 37 | S | F | F | Y | G | G | C | L | G | N | K | N | N | F | L | T | A | K | Y | | | |
| 38 | T | F | F | Y | G | G | C | G | G | N | K | N | N | F | V | R | E | K | Y | | | |
| 39 | T | F | F | Y | G | G | C | M | G | N | K | N | N | F | V | R | E | K | Y | | | |
| 40 | T | F | F | Y | G | G | S | M | G | N | K | N | N | F | V | R | E | K | Y | | | |
| 41 | P | F | F | Y | G | G | C | L | G | N | R | N | N | Y | V | R | E | K | Y | | | |
| 42 | T | F | F | Y | G | G | C | L | G | N | R | N | N | F | V | R | E | K | Y | | | |
| 43 | T | F | F | Y | G | G | C | L | G | N | K | N | N | Y | V | R | E | K | Y | | | |
| 44 | T | F | F | Y | G | G | C | G | G | N | G | N | N | F | L | T | A | K | Y | | | |
| 45 | T | F | F | Y | G | G | C | R | G | N | R | N | N | F | L | T | A | E | Y | | | |
| 46 | T | F | F | Y | G | G | C | R | G | N | G | N | N | F | K | S | A | E | Y | | | |
| 47 | P | F | F | Y | G | G | C | L | G | N | K | N | N | F | K | T | A | E | Y | | | |
| 48 | T | F | F | Y | G | G | C | R | G | N | R | N | N | F | K | T | E | E | Y | | | |
| 49 | T | F | F | Y | G | G | C | R | G | K | R | N | N | F | K | T | E | E | D | | | |
| 50 | P | F | F | Y | G | G | C | G | G | N | G | N | N | F | V | R | E | K | Y | | | |
| 51 | S | F | F | Y | G | G | C | M | G | N | G | N | N | F | V | R | E | K | Y | | | |
| 52 | P | F | F | Y | G | G | C | G | G | N | G | N | N | F | L | R | E | K | Y | | | |
| 53 | T | F | F | Y | G | G | C | L | G | N | G | N | N | F | V | R | E | K | Y | | | |
| 54 | S | F | F | Y | G | G | C | L | G | N | G | N | N | Y | L | R | E | K | Y | | | |
| 55 | T | F | F | Y | G | G | S | L | G | N | G | N | N | F | V | R | E | K | Y | | | |
| 56 | T | F | F | Y | G | G | C | R | G | N | G | N | N | F | V | T | A | E | Y | | | |
| 57 | T | F | F | Y | G | G | C | L | G | K | G | N | N | F | V | S | A | E | Y | | | |
| 58 | T | F | F | Y | G | G | C | L | G | N | R | N | N | F | D | R | A | E | Y | | | |
| 59 | T | F | F | Y | G | G | C | L | G | N | R | N | N | F | L | R | E | E | Y | | | |
| 60 | T | F | F | Y | G | G | C | L | G | N | K | N | N | Y | L | R | E | E | Y | | | |
| 61 | P | F | F | Y | G | G | C | G | G | N | R | N | N | Y | L | R | E | E | Y | | | |
| 62 | P | F | F | Y | G | G | S | G | G | N | R | N | N | Y | L | R | E | E | Y | | | |
| 63 | M | R | P | D | F | C | L | E | P | P | Y | T | G | P | C | V | A | R | I | | | |
| 64 | A | R | I | I | R | Y | F | Y | N | A | K | A | G | L | C | Q | T | F | V | Y | G | |
| 65 | Y | G | G | C | R | A | K | R | N | N | Y | K | S | A | E | D | C | M | R | T | C | G |
| 66 | P | D | F | C | L | E | P | P | Y | T | G | P | C | V | A | R | I | I | R | Y | F | Y |
| 67 | T | F | F | Y | G | G | C | R | G | K | R | N | N | F | K | T | E | E | Y | | | |
| 68 | K | F | F | Y | G | G | C | R | G | K | R | N | N | F | K | T | E | E | Y | | | |
| 69 | T | F | Y | Y | G | G | C | R | G | K | R | N | N | Y | K | T | E | E | Y | | | |
| 70 | T | F | F | Y | G | G | S | R | G | K | R | N | N | F | K | T | E | E | Y | | | |
| 71 | C | T | F | F | Y | G | C | C | R | G | K | R | N | N | F | K | T | E | E | Y | | |
| 72 | T | F | F | Y | G | G | C | R | G | K | R | N | N | F | K | T | E | E | Y | C | | |
| 73 | C | T | F | F | Y | G | S | C | R | G | K | R | N | N | F | K | T | E | E | Y | | |
| 74 | T | F | F | Y | G | G | S | R | G | K | R | N | N | F | K | T | E | E | Y | C | | |
| 75 | P | F | F | Y | G | G | C | R | G | K | R | N | N | F | K | T | E | E | Y | | | |
| 76 | T | F | F | Y | G | G | C | R | G | K | R | N | N | F | K | T | K | E | Y | | | |
| 77 | T | F | F | Y | G | G | K | R | G | K | R | N | N | F | K | T | E | E | Y | | | |
| 78 | T | F | F | Y | G | G | C | R | G | K | R | N | N | F | K | T | K | R | Y | | | |
| 79 | T | F | F | Y | G | G | K | R | G | K | R | N | N | F | K | T | A | E | Y | | | |
| 80 | T | F | F | Y | G | G | K | R | G | K | R | N | N | F | K | T | A | G | Y | | | |
| 81 | T | F | F | Y | G | G | K | R | G | K | R | N | N | F | K | R | E | K | Y | | | |
| 82 | T | F | F | Y | G | G | K | R | G | K | R | N | N | F | K | R | A | K | Y | | | |
| 83 | T | F | F | Y | G | G | C | L | G | N | R | N | N | F | K | T | E | E | Y | | | |
| 84 | T | F | F | Y | G | C | G | R | G | K | R | N | N | F | K | T | E | E | Y | | | |
| 85 | T | F | F | Y | G | G | R | C | G | K | R | N | N | F | K | T | E | E | Y | | | |
| 86 | T | F | F | Y | G | G | C | L | G | N | G | N | N | F | D | T | E | E | E | | | |
| 87 | T | F | Q | Y | G | G | C | R | G | K | R | N | N | F | K | T | E | E | Y | | | |
| 88 | Y | N | K | E | F | G | I | F | N | I | K | G | C | E | R | G | Y | R | F | | | |
| 89 | R | F | K | Y | G | G | C | L | G | N | M | N | N | F | E | T | L | E | E | | | |
| 90 | R | F | K | Y | G | G | C | L | G | N | K | N | N | F | L | R | L | K | Y | | | |
| 91 | R | F | K | Y | G | G | C | L | G | N | K | N | N | Y | L | R | L | K | Y | | | |
| 92 | K | T | K | R | K | R | K | K | Q | R | V | K | I | A | Y | E | E | I | F | K | N | Y |
| 93 | K | T | K | R | K | R | K | K | Q | R | V | K | I | A | Y | | | | | | | |
| 94 | R | G | G | R | L | S | Y | S | R | R | F | S | T | S | T | G | R | | | | | |
| 95 | R | R | L | S | Y | S | R | R | R | F | | | | | | | | | | | | |
| 96 | R | Q | I | K | I | W | F | Q | N | R | R | M | K | W | K | K | | | | | | |
| 97 | T | F | F | Y | G | G | S | R | G | K | R | N | N | F | K | T | E | E | Y | | | |
| 98 | M | R | P | D | F | C | L | E | P | P | Y | T | G | P | C | V | A | R | I | | | |
| | I | R | Y | F | Y | N | A | K | A | G | L | C | Q | T | F | V | Y | G | G | | | |
| | C | R | A | K | R | N | N | F | K | S | A | E | D | C | M | R | T | C | G | G | A | |
| 99 | T | F | F | Y | G | G | C | R | G | K | R | N | N | F | K | T | K | E | Y | | | |
| 100 | R | F | K | Y | G | G | C | L | G | N | K | N | N | Y | L | R | L | K | Y | | | |
| 101 | T | F | F | Y | G | G | C | R | A | K | R | N | N | F | K | R | A | K | Y | | | |
| 102 | N | A | K | A | G | L | C | Q | T | F | V | Y | G | G | C | L | A | K | R | N | N | F |
| | E | S | A | E | D | C | M | R | T | C | G | G | A | | | | | | | | | |
| 103 | Y | G | G | C | R | A | K | R | N | N | F | K | S | A | E | D | C | M | R | T | C | G |
| | G | A | | | | | | | | | | | | | | | | | | | | |
| 104 | G | L | C | Q | T | F | V | Y | G | G | C | R | A | K | R | N | N | F | K | S | A | E |
| 105 | L | C | Q | T | F | V | Y | G | G | C | E | A | K | R | N | N | F | K | S | A | | |
| 107 | T | F | F | Y | G | G | S | R | G | K | R | N | N | F | K | T | E | E | Y | | | |
| 108 | R | F | F | Y | G | G | S | R | G | K | R | N | N | F | K | T | E | E | Y | | | |
| 109 | R | F | F | Y | G | G | S | R | G | K | R | N | N | F | K | T | E | E | Y | | | |
| 110 | R | F | F | Y | G | G | S | R | G | K | R | N | N | F | R | T | E | E | Y | | | |
| 111 | T | F | F | Y | G | G | S | R | G | K | R | N | N | F | R | T | E | E | Y | | | |
| 112 | T | F | F | Y | G | G | S | R | G | R | R | N | N | F | R | T | E | E | Y | | | |
| 113 | C | T | F | F | Y | G | G | S | R | G | K | R | N | N | F | K | T | E | E | Y | | |
| 114 | T | F | F | Y | G | G | S | R | G | K | R | N | N | F | K | T | E | E | Y | C | | |
| 115 | C | T | F | F | Y | G | G | S | R | G | R | R | N | N | F | R | T | E | E | Y | | |
| 116 | T | F | F | Y | G | G | S | R | G | R | R | N | N | F | R | T | E | E | Y | C | | |
| 117 | Y | E | E | T | K | F | N | N | R | K | G | R | S | G | G | Y | F | F | T | | | |

Polypeptides Nos. 5, 67, 76, and 91, include the sequences of SEQ ID NOS: 5, 67, 76, and 91, respectively, and are amidated at the C-terminus
Polypeptides Nos. 107, 109, and 110 include the sequences of SEQ ID NOS: 97, 109, and 110, respectively, and are acetylated at the N-terminus

In any of the above aspects, the targeting moiety may include an amino acid sequence having the formula:

X1-X2-X3-X4-X5-X6-X7-X8-X9-X10-X11-X12-X13-X14-X15-X16-X17-X18-X19 where each of X1-X19 (e.g., X1-X6, X8, X9, X11-X14, and X16-X19) is, independently, any amino acid (e.g., a naturally occurring amino acid such as Ala, Arg, Asn, Asp, Cys, Gln, Glu, Gly, His, Ile, Leu, Lys, Met, Phe, Pro, Ser, Thr, Trp, Tyr, and Val) or absent and at least one (e.g., 2 or 3) of X1, X10, and X15 is arginine. In some embodiments, X7 is Ser or Cys; or X10 and X15 each are independently Arg or Lys. In some embodiments, the residues from X1 through X19, inclusive, are substantially identical to any of the amino acid sequences of any one of SEQ ID NOS:1-105 and 107-116 (e.g., Angiopep-1, Angiopep-2, Angiopep-3, Angiopep-4a, Angiopep-4b, Angiopep-5, Angiopep-6, and Angiopep-7). In some embodiments, at least one (e.g., 2, 3, 4, or 5) of the amino acids X1-X19 is Arg. In some embodiments, the polypeptide has one or more additional cysteine residues at the N-terminal of the polypeptide, the C-terminal of the polypeptide, or both.

In any of the above aspects, the targeting moiety may include the amino acid sequence Lys-Arg-X3-X4-X5-Lys (formula Ia), where X3 is Asn or Gln; X4 is Asn or Gln; and X5 is Phe, Tyr, or Trp; where the polypeptide is optionally fewer than 200 amino acids in length (e.g., fewer than 150, 100, 75, 50, 45, 40, 35, 30, 25, 20, 19, 18, 17, 16, 15, 14, 12, 10, 11, 8, or 7 amino acids, or any range between these numbers); where the polypeptide optionally includes one or more D-isomers of an amino acid recited in formula Ia (e.g., a D-isomer of Lys, Arg, X3, X4, X5, or Lys); and where the polypeptide is not a peptide in Table 2.

In any of the above aspects, the targeting moiety may include the amino acid sequence Lys-Arg-X3-X4-X5-Lys (formula Ia), where X3 is Asn or Gln; X4 is Asn or Gln; and X5 is Phe, Tyr, or Trp; where the polypeptide is fewer than 19 amino acids in length (e.g., fewer than 18, 17, 16, 15, 14, 12, 10, 11, 8, or 7 amino acids, or any range between these numbers); and where the polypeptide optionally includes one or more D-isomers of an amino acid recited in formula Ia (e.g., a D-isomer of Lys, Arg, X3, X4, X5, or Lys).

In any of the above aspects, the targeting moiety may include the amino acid sequence of Z1-Lys-Arg-X3-X4-X5-Lys-Z2 (formula Ib), where X3 is Asn or Gln; X4 is Asn or Gln; X5 is Phe, Tyr, or Trp; Z1 is absent, Cys, Gly, Cys-Gly, Arg-Gly, Cys-Arg-Gly, Ser-Arg-Gly, Cys-Ser-Arg-Gly, Gly-Ser-Arg-Gly, Cys-Gly-Ser-Arg-Gly, Gly-Gly-Ser-Arg-Gly, Cys-Gly-Gly-Ser-Arg-Gly, Tyr-Gly-Gly-Ser-Arg-Gly, Cys-Tyr-Gly-Gly-Ser-Arg-Gly, Phe-Tyr-Gly-Gly-Ser-Arg-Gly, Cys-Phe-Tyr-Gly-Gly-Ser-Arg-Gly, Phe-Phe-Tyr-Gly-Gly-Ser-Arg-Gly, Cys-Phe-Phe-Tyr-Gly-Gly-Ser-Arg-Gly, Thr-Phe-Phe-Tyr-Gly-Gly-Ser-Arg-Gly, or Cys-Thr-Phe-Phe-Tyr-Gly-Gly-Ser-Arg-Gly; and Z2 is absent, Cys, Tyr, Tyr-Cys, Cys-Tyr, Thr-Glu-Glu-Tyr, or Thr-Glu-Glu-Tyr-Cys; and where the polypeptide optionally comprises one or more D-isomers of an amino acid recited in formula Ib, Z1, or Z2.

In any of the above aspects, the targeting moiety may include the amino acid sequence Lys-Arg-Asn-Asn-Phe-Lys. In other embodiments, the targeting moiety has an amino acid sequence of Lys-Arg-Asn-Asn-Phe-Lys-Tyr. In still other embodiments, the targeting moiety has an amino acid sequence of Lys-Arg-Asn-Asn-Phe-Lys-Tyr-Cys.

In any of the above aspects, the targeting moiety may have the amino acid sequence of X1-X2-Asn-Asn-X5-X6 (formula IIa), where X1 is Lys or D-Lys; X2 is Arg or D-Arg; X5 is Phe or D-Phe; and X6 is Lys or D-Lys; and where at least one (e.g., at least two, three, or four) of X1, X2, X5, or X6 is a D-amino acid.

In any of the above aspects, the targeting moiety may have the amino acid sequence of X1-X2-Asn-Asn-X5-X6-X7 (formula IIb), where X1 is Lys or D-Lys; X2 is Arg or D-Arg; X5 is Phe or D-Phe; X6 is Lys or D-Lys; and X7 is Tyr or D-Tyr; and where at least one (e.g., at least two, three, four, or five) of X1, X2, X5, X6, or X7 is a D-amino acid.

In any of the above aspects, the targeting moiety may have the amino acid sequence of Z1-Lys-Arg-X3-X4-X5-Lys-Z2 (formula IIc), where X3 is Asn or Gln; X4 is Asn or Gln; X5 is Phe, Tyr, or Trp; Z1 is absent, Cys, Gly, Cys-Gly, Arg-Gly, Cys-Arg-Gly, Ser-Arg-Gly, Cys-Ser-Arg-Gly, Gly-Ser-Arg-Gly, Cys-Gly-Ser-Arg-Gly, Gly-Gly-Ser-Arg-Gly, Cys-Gly-Gly-Ser-Arg-Gly, Tyr-Gly-Gly-Ser-Arg-Gly, Cys-Tyr-Gly-Gly-Ser-Arg-Gly, Phe-Tyr-Gly-Gly-Ser-Arg-Gly, Cys-Phe-Tyr-Gly-Gly-Ser-Arg-Gly, Phe-Phe-Tyr-Gly-Gly-Ser-Arg-Gly, Cys-Phe-Phe-Tyr-Gly-Gly-Ser-Arg-Gly, Thr-Phe-Phe-Tyr-Gly-Gly-Ser-Arg-Gly, or Cys-Thr-Phe-Phe-Tyr-Gly-Gly-Ser-Arg-Gly; and Z2 is absent, Cys, Tyr, Tyr-Cys, Cys-Tyr, Thr-Glu-Glu-Tyr, or Thr-Glu-Glu-Tyr-Cys; where at least one of X1, X2, X5, X6, or X7 is a D-amino acid; and where the polypeptide optionally comprises one or more D-isomers of an amino acid recited in Z1 or Z2.

In any of the above aspects, the targeting moiety may have the amino acid sequence of Thr-Phe-Phe-Tyr-Gly-Gly-Ser-Arg-Gly-Lys-Arg-Asn-Asn-Phe-Lys-Thr-Glu-Glu-Tyr (An2), where any one or more amino acids are D-isomers. For example, the targeting moiety can have 1, 2, 3, 4, or 5 amino acids which are D-isomers. In a preferred embodiment, one or more or all of positions 8, 10, and 11 can be D-isomers. In yet another embodiment, one or more or all of positions 8, 10, 11, and 15 can have D-isomers.

In any of the above aspects, the targeting moiety may be Thr-Phe-Phe-Tyr-Gly-Gly-Ser-D-Arg-Gly-D-Lys-D-Arg-Asn-Asn-Phe-Lys-Thr-Glu-Glu-Tyr (3D-An2); Phe-Tyr-Gly-Gly-Ser-Arg-Gly-Lys-Arg-Asn-Asn-Phe-Lys-Thr-Glu-Glu-Tyr-Cys (P1); Phe-Tyr-Gly-Gly-Ser-Arg-Gly-D-Lys-D-Arg-Asn-Asn-D-Phe-Lys-Thr-Glu-Glu-Tyr-Cys (P1a); Phe-Tyr-Gly-Gly-Ser-Arg-Gly-D-Lys-D-Arg-Asn-Asn-D-Phe-D-Lys-Thr-Glu-Glu-Tyr-Cys (P1b); Phe-Tyr-Gly-Gly-Ser-Arg-Gly-D-Lys-D-Arg-Asn-Asn-D-Phe-D-Lys-Thr-Glu-Glu-D-Tyr-Cys (P1c); D-Phe-D-Tyr-Gly-Gly-Ser-D-Arg-Gly-D-Lys-D-Arg-Asn-Asn-D-Phe-D-Lys-Thr-Glu-D-Glu-D-Tyr-Cys (P1d); Gly-Gly-Ser-Arg-Gly-Lys-Arg-Asn-Asn-Phe-Lys-Thr-Glu-Glu-Tyr-Cys (P2); Ser-Arg-Gly-Lys-Arg-Asn-Asn-Phe-Lys-Thr-Glu-Glu-Tyr-Cys (P3); Gly-Lys-Arg-Asn-Asn-Phe-Lys-Thr-Glu-Glu-Tyr-Cys (P4); Lys-Arg-Asn-Asn-Phe-Lys-Thr-Glu-Glu-Tyr-Cys (P5); D-Lys-D-Arg-Asn-Asn-D-Phe-Lys-Thr-Glu-Glu-Tyr-Cys (P5a); D-Lys-D-Arg-Asn-Asn-D-Phe-D-Lys-Thr-Glu-Glu-Tyr-Cys (P5b); D-Lys-D-Arg-Asn-Asn-D-Phe-D-Lys-Thr-Glu-Glu-D-Tyr-Cys (P5c); Lys-Arg-Asn-Asn-Phe-Lys-Tyr-Cys (P6); D-Lys-D-Arg-Asn-Asn-D-Phe-Lys-Tyr-Cys (P6a); D-Lys-D-Arg-Asn-Asn-D-Phe-D-Lys-Tyr-Cys (P6b); Thr-Phe-Phe-Tyr-Gly-Gly-Ser-D-Arg-Gly-D-Lys-D-Arg-Asn-Asn-Phe-D-Lys-Thr-Glu-Glu-Tyr; and D-Lys-D-Arg-Asn-Asn-D-Phe-D-Lys-D-Tyr-Cys (P6c); or a fragment thereof. In other embodiments, the targeting moiety has a sequence of one of the aforementioned peptides having from 0 to 5 (e.g., from 0 to 4, 0 to 3, 0 to 2, 0 to 1, 1 to 5, 1 to 4, 1 to 3, 1 to 2, 2 to 5, 2 to 4, 2 to 3, 3 to 5, 3 to 4, or 4 to 5) substitutions, deletions, or additions of amino acids.

In any of the above aspects, the polypeptide may be Phe-Tyr-Gly-Gly-Ser-Arg-Gly-Lys-Arg-Asn-Asn-Phe-Lys-Thr-Glu-Glu; Gly-Gly-Ser-Arg-Gly-Lys-Arg-Asn-Asn-Phe-Lys-Thr-Glu-Glu; Ser-Arg-Gly-Lys-Arg-Asn-Asn-Phe-Lys-Thr-Glu-Glu; Gly-Lys-Arg-Asn-Asn-Phe-Lys-Thr-Glu-Glu; Lys-Arg-Asn-Asn-Phe-Lys-Thr-Glu-Glu; or Lys-Arg-Asn-Asn-Phe-Lys, or a fragment thereof.

In any of the above aspects, the polypeptide may be Thr-Phe-Phe-Tyr-Gly-Gly-Ser-D-Arg-Gly-D-Lys-D-Arg-Asn-Asn-Phe-Lys-Thr-Glu-Glu-Tyr (3D-An2); Phe-Tyr-Gly-Gly-Ser-Arg-Gly-Lys-Arg-Asn-Asn-Phe-Lys-Thr-Glu-Glu-Tyr-Cys (P1); Phe-Tyr-Gly-Gly-Ser-Arg-Gly-D-Lys-D-Arg-Asn-Asn-D-Phe-Lys-Thr-Glu-Glu-Tyr-Cys (P1a); Phe-Tyr-Gly-Gly-Ser-Arg-Gly-D-Lys-D-Arg-Asn-Asn-D-Phe-D-Lys-Thr-Glu-Glu-Tyr-Cys (P1b); Phe-Tyr-Gly-Gly-Ser-Arg-Gly-D-Lys-D-Arg-Asn-Asn-D-Phe-D-Lys-Thr-Glu-Glu-D-Tyr-Cys (P1c); D-Phe-D-Tyr-Gly-Gly-Ser-D-Arg-Gly-D-Lys-D-Arg-Asn-Asn-D-Phe-D-Lys-Thr-Glu-D-Glu-D-Tyr-Cys (P1d) or a fragment thereof (e.g., deletion of 1 to 7 amino acids from the N-terminus of P1, P1a, P1b, P1c, or P1d; a deletion of 1 to 5 amino acids from the C-terminus of P1, P1a, P1b, P1c, or P1d; or deletions of 1 to 7 amino acids from the N-terminus of P1, P1a, P1b, P1c, or P1d and 1 to 5 amino acids from the C-terminus of P1, P1a, P1b, P1c, or P1d).

In any of the targeting moieties described herein, the moiety may include additions or deletions of 1, 2, 3, 4, or 5 amino acids (e.g., from 1 to 3 amino acids) may be made from an amino acid sequence described herein (e.g., from Lys-Arg-X3-X4-X5-Lys).

In any of the targeting moieties described herein, the moiety may have one or more additional cysteine residues at the N-terminal of the polypeptide, the C-terminal of the polypeptide, or both. In other embodiments, the targeting moiety may have one or more additional tyrosine residues at the N-terminal of the polypeptide, the C-terminal of the polypeptide, or both. In yet further embodiments, the targeting moiety has the amino acid sequence Tyr-Cys and/or Cys-Tyr at the N-terminal of the polypeptide, the C-terminal of the polypeptide, or both.

In certain embodiments of any of the above aspects, the targeting moiety may be fewer than 15 amino acids in length (e.g., fewer than 10 amino acids in length).

In certain embodiments of any of the above aspects, the targeting moiety may have a C-terminus that is amidated. In other embodiments, the targeting moiety is efficiently transported across the BBB (e.g., is transported across the BBB more efficiently than Angiopep-2).

In certain embodiments of any of the above aspects, the fusion protein, targeting moiety, or lysosomal enzyme (e.g., IDS), fragment, or analog is modified (e.g., as described herein). The fusion protein, targeting moiety, or lysosomal enzyme, fragment, or analog may be amidated, acetylated, or both. Such modifications may be at the amino or carboxy terminus of the polypeptide. The fusion protein, targeting moiety, or lysosomal enzyme, fragment, or analog may also include or be a peptidomimetic (e.g., those described herein) of any of the polypeptides described herein. The fusion protein, targeting moiety, or lysosomal enzyme, fragment, or analog may be in a multimeric form, for example, dimeric form (e.g., formed by disulfide bonding through cysteine residues).

In certain embodiments, the targeting moiety, lysosomal enzyme (e.g., IDS), enzyme fragment, or enzyme analog has an amino acid sequence described herein with at least one amino acid substitution (e.g., 2, 3, 4, 5, 6, 7, 8, 9, 10, 11, or 12 substitutions), insertion, or deletion. The polypeptide may contain, for example, 1 to 12, 1 to 10, 1 to 5, or 1 to 3 amino acid substitutions, for example, 1 to 10 (e.g., to 9, 8, 7, 6, 5, 4, 3, 2) amino acid substitutions. The amino acid substitution(s) may be conservative or non-conservative. For example, the targeting moiety may have an arginine at one, two, or three of the positions corresponding to positions 1, 10, and 15 of the amino acid sequence of any of SEQ ID NO:1, Angiopep-1, Angiopep-2, Angiopep-3, Angiopep-4a, Angiopep-4b, Angiopep-5, Angiopep-6, and Angiopep-7.

In any of the above aspects, the compound may specifically exclude a polypeptide including or consisting of any of SEQ ID NOS:1-105 and 107-117 (e.g., Angiopep-1, Angiopep-2, Angiopep-3, Angiopep-4a, Angiopep-4b, Angiopep-5, Angiopep-6, and Angiopep-7). In some embodiments, the polypeptides and conjugates of the invention exclude the polypeptides of SEQ ID NOS:102, 103, 104, and 105.

In any of the above aspects, the linker (X) may be any linker known in the art or described herein. In particular embodiments, the linker is a covalent bond (e.g., a peptide bond), a chemical linking agent (e.g., those described herein), an amino acid or a peptide (e.g., 2, 3, 4, 5, 8, 10, or more amino acids).

In certain embodiments, the linker has the formula:

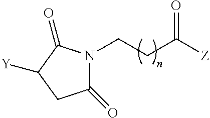

where n is an integer between 2 and 15 (e.g., 2, 3, 4, 5, 6, 7, 8, 9, 10, 11, 12, 13, 14, or 15); and either Y is a thiol on A and Z is a primary amine on B or Y is a thiol on B and Z is a primary amine on A. In certain embodiments, the linker is an N-Succinimidyl (acetylthio)acetate (SATA) linker or a hydrazide linker. The linker may be conjugated to the enzyme (e.g., IDS) or the targeting moiety (e.g., Angiopep-2), through a free amine, a cysteine side chain (e.g., of Angiopep-2-Cys or Cys-Angiopep-2), or through a glycosylation site.

In certain embodiments, the compound has the formula

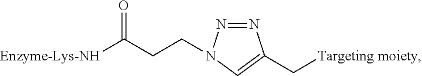

where the “Lys-NH” group represents either a lysine present in the enzyme or an N-terminal or C-terminal lysine. In another example, the compound has the structure:

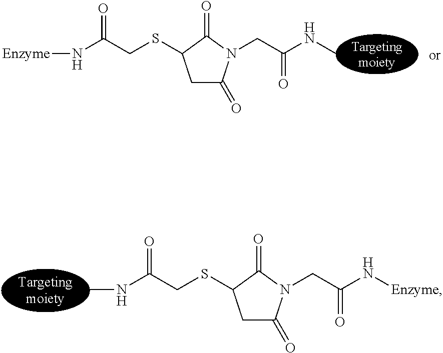

where each —NH— group represents a primary amino present on the targeting moiety and the enzyme, respectively. In particular embodiments, The enzyme may be IDS or the targeting moiety may be Angiopep-2.

In certain embodiments, the compound is a fusion protein including the targeting moiety (e.g., Angiopep-2) and the lysosomal enzyme (e.g., IDS), enzyme fragment, or enzyme analog.

In certain embodiments, the linker includes a click-chemistry reaction pair selected from the group consisting of a Huisgen 1,3-dipolar cycloaddition reaction between an alkynyl group and an azido group to form a triazole-containing linker; a Diels-Alder reaction between a diene having a 4π electron system (e.g., an optionally substituted 1,3-unsaturated compound, such as optionally substituted 1,3-butadiene, 1-methoxy-3-trimethylsilyloxy-1,3-butadiene, cyclopentadiene, cyclohexadiene, or furan) and a dienophile or heterodienophile having a 2π electron system (e.g., an optionally substituted alkenyl group or an optionally substituted alkynyl group); a ring opening reaction with a nucleophile and a strained heterocyclyl electrophile; and a splint ligation reaction with a phosphorothioate group and an iodo group; and a reductive amination reaction with an aldehyde group and an amino group. In one aspect of the invention, the linker is selected from the group consisting of monofluorocyclooctyne (MFCO), difluorocyclooctyne (DFCO), cyclooctyne (OCT), dibenzocyclooctyne (DIBO), biarylazacyclooctyne (BARAC), difluorobenzocyclooctyne (DIFBO), and bicyclo[6.1.0]nonyne (BCN). In another aspect, the linker is a maleimide group or an S-acetylthioacetate (SATA) group. The peptide targeting moiety is attached to the linker via an N-terminal azido group or a C-terminal azido group.

In one embodiment, the compound includes an Angiopep-2 joined to IDS via a BCN linker. This compound can have the general structure

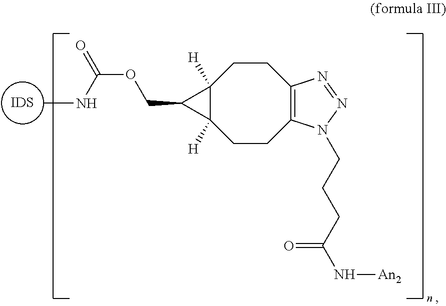

where n is the number of Angiopep-2 moieties attached to IDS via the linker and is between 1 to 6, An₂ is Angiopep-2, the NH group attached to An2 is the N-terminus amino group of Angiopep-2, and the NH group attached to IDS represents the side chain primary amino group from a lysine in IDS. The compound can also have the structure

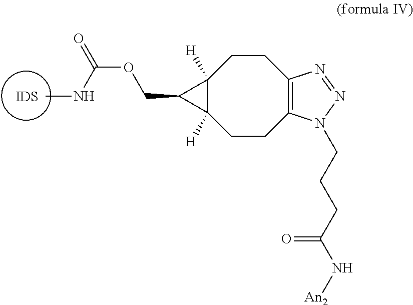

The compound can also have the structure

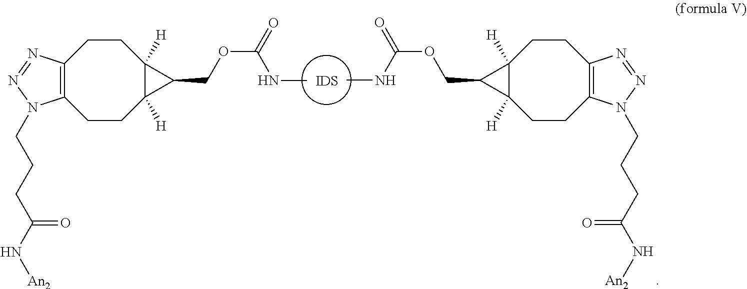

In each of the above formulae, An₂ is Angiopep-2, the NH group attached to An2 is the N-terminus amino group of Angiopep-2, and the NH group attached to IDS represents the side chain primary amino group from a lysine in IDS.

In any of the aspects of the compounds of the invention, Angiopep-2 can be derivatized with an azide group at the N- or C-terminus of the polypeptide, such that the azide group can be reacted with an alkyne derivatized linker, in a click-chemistry reaction, to attach the Angiopep-2 to the linker. The invention also features a composition comprising a compound of formula III where an average value of n is between 1 and 6 (e.g., 1, 1.5, 2, 2.5, 3, 3.5, 4, 4.5, 5, 5.5, or 6).

The compound with a BCN linker can also have the structure

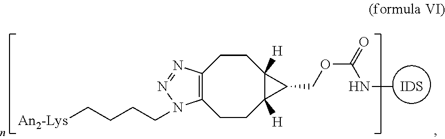

where n is the number of Angiopep-2 moieties attached to IDS via the linker and is between 1 to 6, An₂ is Angiopep-2 and is attached to the linker via the side chain primary amino group of a lysine at the C-terminus of Angiopep-2, and the NH group attached to IDS represents the side chain primary amino group from a lysine in IDS.

The invention features a composition including a compound of formula VI where an average value of n is between 1 and 6 (e.g., 1, 1.5, 2, 2.3, 2.5, 3, 3.5, 4, 4.5, 5, 5.5, or 6).

In one embodiment, the compound includes an Angiopep-2 joined to IDS via a MFCO linker. The Angiopep-2 can be joined to the MFCO linker via the N-terminus amino group of Angiopep-2. The compound can have the structure

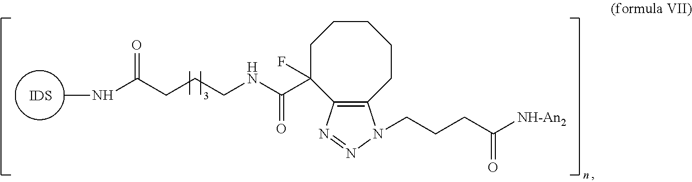

where n is the number of Angiopep-2 moieties attached to IDS via the linker and is between 1 to 6, An₂ is Angiopep-2, the NH group attached to An2 is the N-terminus amino group of Angiopep-2, and the NH group attached to IDS represents the side chain primary amino group from a lysine in IDS.

The invention also features a composition including the compound of formula VII where the average value of n is between 1 and 6 (e.g., 1, 1.5, 2, 2.5, 2.6, 3, 3.5, 4, 4.4, 4.5, 5, 5.3, 5.5, or 6).

In one aspect of the invention, Angiopep-2 is joined to the MFCO linker via the side chain primary amino group of an amino acid (e.g., a lysine) at the C-terminus of Angiopep-2 and the compound has the structure

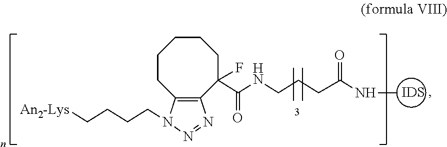

where n is the number of Angiopep-2 moieties attached to IDS via the linker and is between 1 to 6, An₂ is Angiopep-2 and is attached to the linker via the side chain primary amino group of a lysine at the C-terminus of Angiopep-2, and the NH group attached to IDS represents the side chain primary amino group from a lysine in IDS. The invention features a composition including the compound of formula VIII where the average value of n is between 1 and 6 (e.g., 1, 1.5, 2, 2.5, 3, 3.5, 4, 4.5, 4.9, 5, 5.5, or 6).

In another embodiment of the invention, the compound includes Angiopep-2 joined to IDS via a DBCO linker and has the structure

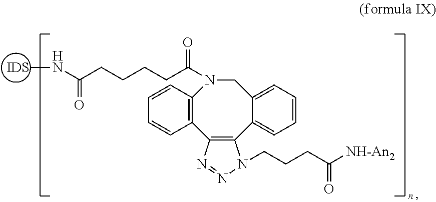

where n is the number of Angiopep-2 moieties attached to IDS via the linker and is between 1 to 6, An₂ is Angiopep-2, the NH group attached to An2 is the N-terminus amino group of Angiopep-2, and the NH group attached to IDS represents the side chain primary amino group from a lysine in IDS. The invention features a composition including the compound of formula IX where the average value of n is between 1 and 6 (e.g., 1, 1.3, 1.5, 2, 2.5, 3, 3.5, 4, 4.5, 5, 5.5, or 6).

The invention also features a compound where Angiopep-2-Cys is joined to IDS via a maleimide group and has the structure

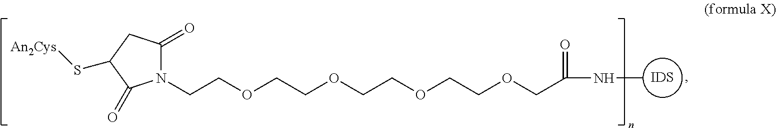

where n is the number of Angiopep-2 moieties attached to IDS via the linker and is between 1 to 6, wherein An₂Cys, the S moiety attached to An₂Cys represents the side chain sulfide on the cysteine in Angiopep-2-Cys, and the NH group attached to IDS represents the side chain primary amino group from a lysine in IDS. The invention features a composition including the compound of formula X where the average value of n is between 0.5 and 6 (e.g., 0.5, 0.8, 1, 1.5, 2, 2.5, 3, 3.5, 4, 4.5, 5, 5.5, or 6).

In an alternate embodiment, Cys-Angiopep-2 is joined to IDS via a maleimide group and has the structure

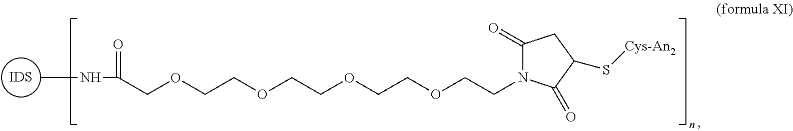

where n is the number of Angiopep-2 moieties attached to IDS via the linker and is between 1 to 6, wherein Cys-An₂ is Cys-Angiopep-2, the S moiety attached to Cys-An₂ represents the side chain sulfide on the cysteine in Cys-Angiopep-2, and the NH group attached to IDS represents the side chain primary amino group from a lysine in IDS. The invention features a composition including the compound of formula XI where the average value of n is between 0.5 and 6 (e.g., 0.5, 0.9, 1, 1.5, 2, 2.5, 3, 3.5, 4, 4.5, 5, 5.5, or 6).

In one aspect of the above embodiments, the linker can be a maleimide group functionalized with an alkyne group selected from the group consisting of monofluorocyclooctyne (MFCO), difluorocyclooctyne (DFCO), cyclooctyne (OCT), dibenzocyclooctyne (DIBO), biarylazacyclooctyne (BARAC), difluorobenzocyclooctyne (DIFBO), and bicyclo[6.1.0]nonyne (BCN) and the alkyne-functionalized maleimide is attached to an Angiopep-2 via an azido group attached to Angiopep-2.

In one embodiment of the invention, the compound includes Angiopep-2 joined to IDS via an S-acetylthioacetate (SATA) group and has the structure

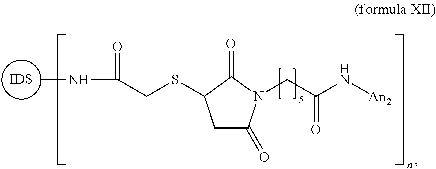

where n is the number of Angiopep-2 moieties attached to IDS via the linker and is between 1-6, An₂ is Angiopep-2, the NH group attached to An2 is the N-terminus amino group of Angiopep-2, and the NH group attached to IDS represents the side chain primary amino group from a lysine in IDS. The invention features a composition comprising the compound of formula XII where the average value of n is between 1 and 6 (e.g., 1, 1.5, 2, 2.5, 2.6, 3, 3.5, 4, 4.5, 5, 5.5, or 6).

The compounds described above can have 1, 2, 3, 4, 5, or more peptide targeting moieties attached to the enzyme via a linker, where the targeting moiety is Angiopep-2 and the enzyme is a lysosomal enzyme, e.g., IDS.

The invention also features compositions that include the compounds that are represented by the above formulae, where the average number of Angiopep-2 moieties attached to each IDS is between 1-6 (e.g., 1, 1.5, 2, 2.5, 3, 3.5, 4, 4.5, 5, 5.5, or 6), preferably, between 1.5-5, more preferably between 2-4. In some aspects of the above composition, the average number of Angiopep-2 moieties attached to each IDS can be about 2 (e.g., 1, 1.5, 2, 2.5, or 3). More preferably, the average number of Angiopep-2 moieties attached to each IDS can be about 4 (e.g., 2, 2.5, 3, 3.5, 4, 4.5, or 5). Alternatively, the average number of Angiopep-2 moieties attached to each IDS can be about 6 (e.g., 3.5, 4, 4.5, 5, 5.5, 6, 6.5, or 7).

The invention features a composition that includes nanoparticles which are conjugated to any of the compounds described above. The invention also features a liposome formulation of any of the compounds featured above.

The invention features a pharmaceutical composition that includes any one of the compounds described above and a pharmaceutically acceptable carrier. The invention also features a method of treating or treating prophylactically a subject having a lysosomal storage disorder, where the method includes administering to a subject any of the above described compounds or compositions. In one aspect of the method, the lysosomal storage disorder is mucopolysaccharidosis Type II (MPS-II) and the lysosomal enzyme is IDS. In another aspect of the method, the subject has the severe form of MPS-II or the the attenuated form of MPS-II. In yet another aspect of the method, the subject has neurological symptoms. the subject can start treatment at under five years of age, preferably under three years of age. The subject can be an infant. The methods of the invention also include parenteral administration of the compounds and compositions of the invention.

By “subject” is meant a human or non-human animal (e.g., a mammal).

By “lysosomal enzyme” is meant any enzyme that is found in the lysosome in which a defect in that enzyme can lead to a lysosomal storage disorder.

By “lysosomal storage disorder” is meant any disease caused by a defect in a lysosomal enzyme. Approximately fifty such disorders have been identified.

By “targeting moiety” is meant a compound or molecule such as a polypeptide or a polypeptide mimetic that can be transported into a particular cell type (e.g., liver, lungs, kidney, spleen, or muscle), into particular cellular compartments (e.g., the lysosome), or across the BBB. In certain embodiments, the targeting moiety may bind to receptors present on brain endothelial cells and thereby be transported across the BBB by transcytosis. The targeting moiety may be a molecule for which high levels of transendothelial transport may be obtained, without affecting the cell or BBB integrity. The targeting moiety may be a polypeptide or a peptidomimetic and may be naturally occurring or produced by chemical synthesis or recombinant genetic technology.

By “treating” a disease, disorder, or condition in a subject is meant reducing at least one symptom of the disease, disorder, or condition by administrating a therapeutic agent to the subject.

By “treating prophylactically” a disease, disorder, or condition in a subject is meant reducing the frequency of occurrence of or reducing the severity of a disease, disorder or condition by administering a therapeutic agent to the subject prior to the onset of disease symptoms.

By a polypeptide which is “efficiently transported across the BBB” is meant a polypeptide that is able to cross the BBB at least as efficiently as Angiopep-6 (i.e., greater than 38.5% that of Angiopep-1 (250 nM) in the in situ brain perfusion assay described in U.S. patent application Ser. No. 11/807,597, filed May 29, 2007, hereby incorporated by reference). Accordingly, a polypeptide which is “not efficiently transported across the BBB” is transported to the brain at lower levels (e.g., transported less efficiently than Angiopep-6).

By a polypeptide or compound which is “efficiently transported to a particular cell type” is meant that the polypeptide or compound is able to accumulate (e.g., either due to increased transport into the cell, decreased efflux from the cell, or a combination thereof) in that cell type to at least a 10% (e.g., 25%, 50%, 100%, 200%, 500%, 1,000%, 5,000%, or 10,000%) greater extent than either a control substance, or, in the case of a conjugate, as compared to the unconjugated agent. Such activities are described in detail in International Application Publication No. WO 2007/009229, hereby incorporated by reference.

By “substantial identity” or “substantially identical” is meant a polypeptide or polynucleotide sequence that has the same polypeptide or polynucleotide sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). For nucleic acids, the length of comparison sequences will generally be at least 5, 10, 11, 12, 13, 14, 15, 16, 17, 18, 19, 20, 21, 22, 23, 24, or 25 contiguous nucleotides (e.g., the full-length nucleotide sequence). Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

Other features and advantages of the invention will be apparent from the following Detailed Description, the drawings, and the claims.

### BRIEF DESCRIPTION OF THE DRAWINGS

FIG. 1 is a schematic diagram showing the IDS constructs that were generated.

FIG. 2 is an image showing a western blot of cell culture media from CHO—S cells transfected with the indicated constructs using an anti-IDS antibody.

FIG. 3 is a schematic diagram showing the fluorescence assay used to detect IDS activity in the examples described below.

FIG. 4 is a graph showing IDS activity in cell culture media from CHO—S cells transfected with the indicated constructs.

FIG. 5A is a graph showing IDS activity over a seven-day period following transfection of CHO—S cells with the indicated constructs.

FIG. 5B is a set of western blot images showing the expression of either IDS-His or IDS-An2-His over a seven-day period in CHO—S cells.

FIG. 6A is a graph showing reduction of ³⁵S-GAG accumulation in MPS-II fibroblasts upon treatment with media from CHO—S cells expressing the indicated construct.

FIG. 6B is a graph showing reduction in GAG accumulation in MPS-II fibroblasts upon treatment with purified IDS-An2-His.

FIGS. 7A-7C are sequences of isoforms of IDS (isoform a, FIG. 7A; isoform b; FIG. 7B; isoform c, FIG. 7C).

FIG. 8 is a set of images showing coomassie blue staining and western blot detection of IDS (JR-032) and IDS-Angiopep-2 conjugates.

FIG. 9 is a graph showing the enzyme activity of IDS-Angiopep-2 conjugates compared to JR-032. Enzyme activity is expressed as % JCR-032 control. For conjugates, number of determinations is between 4 and 8, for JR-032, each bar is the average of 15 determinations.

FIG. 10 is a graph showing GAG concentration measured in MPSII patient fibroblasts treated with unconjugated JR-032 or individual conjugates (4 ng/ml). GAG levels are expressed as % of GAG measured in healthy patient fibroblasts.

FIG. 11 is a graph showing that Angiopep-2-IDS conjugates reduce GAG concentration in MPSII fibroblasts with similar potency to unconjugated JR-032. GAG concentration was measured in MPSII patient fibroblasts treated with JR-032 of three conjugates at various concentrations. GAG levels are expressed as % of GAG measured in healthy patient fibroblasts.

FIGS. 12A-12B is a set of graphs showing the distribution of JR-032 in different parts of the brain.

FIG. 13 is a graph showing the brain distribution of unconjugated JR-032 and 15 conjugates respectively at a single time point (2 minutes). Unless the C-terminus is specified, all linkers are connected to An2 by N-terminal attachment.

FIGS. 14A-14D are a set of graphs showing MALDI-TOF analysis of 70-56-1B, 70-56-2B, 68-32-2, and 70-66-1B conjugates.

FIG. 15A shows SEC analysis of 68-32-2, 70-56-1B, 70-56-2B, and 70-66-1B.

FIG. 15B shows SP analysis of 68-32-2, 70-56-1B, 70-56-2B, and 70-66-1B.

FIGS. 16A-16B are a set of graphs showing uptake of Alexa488-IDS and Alexa488-An2-IDS (70-56-2B) by U87 cells in 1 hour and 16 hours respectively.

FIG. 17 is a schematic showing the protocol for measuring intracellular trafficking of Alexa 488 labeled conjugates using confocal microscopy.

FIG. 18 is a set of confocal micrographs showing localization of Alexa-labeled IDS (upper panel) and Alexa-labeled Angiopep-2-IDS (70-56-2B, lower panel) in U87 cells in comparison to lysotracker dye. Colocalization after a 16 hour uptake is shown in fourth panel (merge). Enzymes were incubated at a concentration of 50 nM for 16 hours at 37 C. Magnification is 100×.

FIG. 19 is a set of confocal micrographs showing localization of Alexa-labeled IDS (upper panel) and Alexa-labeled Angiopep-2-IDS (70-56-2B, lower panel) in U87 cells in comparison to lysotracker dye. Lack of colocalization is shown in fourth panel (merge). Enzymes were incubated at a concentration of 100 nM for 1 hour at 37 C. Magnification is 100×.

FIG. 20 is a set of confocal micrographs showing localization of Alexa-labeled IDS (upper panel) and Alexa-labeled Angiopep-2-IDS (70-56-2B, lower panel) in U87 cells in comparison to lysotracker dye. Colocalization is shown in fourth panel (merge) in yellow. Enzymes were incubated at a concentration of 100 nM for 16 hours at 37 C. Magnification is 100×.

FIG. 21 is a confocal micrograph showing localization of Alexa-labeled IDS and Alexa-labeled Angiopep-2-IDS (70-56-1B) in U87 cells in comparison to lysotracker dye. Enzymes were incubated overnight at a concentration of 50 nM at 37 C. Magnification is 100×. The right panel is a zoomed version of the left panel.

FIG. 22 is a set of confocal micrographs showing uptake and localization of Alexa-labeled IDS and Alexa488-labeled An2-IDS conjugates: #68-32-2, 70-66-1B, 70-56-2B, and 68-27-3 in U-87 cells.

FIG. 23 is a graph comparing the brain uptake and distribution of JR-032 and inulin.

FIGS. 24A-24B are graphs comparing the Kᵢₙ and brain distribution of An2-IDS conjugates with that of unconjugated JR-032.

FIGS. 25A-25B are graphs showing that the Angiopep-2-IDS conjugates show increased uptake into U87 cells and that increasing the incorporation ratio of Angiopep-2-IDS conjugates correlates with increased uptake into cells.

FIG. 26 is the amino acid sequence of the IDUA enzyme precursor. The mature enzyme includes amino acids 27-653 of this sequence.

FIG. 27 is a plasmid map of cDNA constructs encoding IDUA fused to Angiopep-2 (An2), and either with or without the histidine (his)-tag. The constructs were subcloned in a suitable expression vector such as pcDNA3.1.

FIG. 28 is a schematic of eight IDUA and EPiC-IDUA fusion proteins.

FIG. 29 is a western blot using anti-IDUA, anti-Angiopep-2, or anti-hexahistidine antibodies, showing the expression levels of IDUA and EPiC-IDUA fusion proteins, as detected in the CHO—S cell media.

FIG. 30A is an image of a Coomassie-stained SDS-PAGE gel showing IDUA and EPiC-IDUA fusion proteins purified from CHO—S media. FIG. 30B is an image of a Coomassie-stained SDS-PAGE gel showing the IDUA-His and An2-IDUA-His proteins with or without removal of the His tag. Below are western blots with anti-His or anti-An2 antibodies to detect the presence or absence of His tag (to confirm removal of His tag) and the presence of the An2 tag.

FIG. 31 is a table showing the protocol for purification of recombinant IDUA in CHO cells.

FIG. 32A is a graph showing the purification profile of IDUA during final step using SP-Sepharose (strong cation-exchange resin). The inset is an image of a Coomassie-stained SDS-PAGE gel showing levels of IDUA in the various fractions during elution. FIG. 32B is a Coomassie-stained SDS-PAGE gel showing the reproducible purification of IDUA and An2-IDUA from various batches with or without the His tag. FIG. 32C is a Coomassie-stained SDS-PAGE gel showing purification of amounts of IDUA and An2-IDUA that are sufficient for in vitro brain perfusion and in vitro assays.

FIG. 33 is a schematic showing the reaction of the IDUA enzyme on the substrate 4-methylumbelliferyl-α-L-iduronide. The substrate is hydrolyzed by IDUA to 4-methylumbelliferone (4-MU), which is detected fluorometrically with a Farrand filter fluorometer using an emission wavelength of 450 nm and an excitation wavelength of 365 nM.

FIG. 34 is a table showing that IDUA-His₈, IDUA, An2-IDUA-His₈, and commercial IDUA-His₁₀ have similar enzymatic activities.

FIG. 35 is a graph showing reduction of GAG by IDUA, IDUA-His, and An2-IDUA-His in MPS-I fibroblasts.

FIG. 36 is a set of graphs showing intra-cellular IDUA activity in MPS-I fibroblasts after exposure to increasing concentrations of IDUA or An2-IDUA enzymes in the cell culture medium.

FIG. 37 is a graph showing the uptake of IDUA proteins by MPS-I fibroblasts in the presence of excess M6P, RAP, or An2.

FIGS. 38A-38C are graphs showing M6P receptor-dependent uptake of IDUA proteins by MPS-I fibroblasts with increasing amounts of An2 (FIG. 13A) and M6P (FIG. 13B). FIG. 13C shows uptake of IDUA and An2-IDUA in presence of increasing amounts of the LRP1 inhibitor, RAP.

FIG. 39A is a set of graphs showing the uptake of IDUA and An2-IDUA (exposed for 2 or 24 hours) by U-87 glioblastoma cells in the presence of An2 peptide (1 mM), M6P (5 mM), and RAP (1 μm) peptide (LRP1 inhibitor). FIG. 39B is a set of western blots showing co-immunoprecipitation of An2-IDUA with LRP1 demonstrating that An2-IDUA interacts with LRP1.

FIG. 40A is a schematic showing the PNGase F cleavage site in IDUA fusion proteins. FIG. 40B are images of Coomassie-stained SDS-PAGE gels showing deglycosylation of non-denatured or denatured An2-IDUA. FIG. 40C is an image of a Coomassie-stained SDS-PAGE gel showing IDUA/or An2-IDUA before and after treatment with PNGase F. FIG. 40D is a graph showing the effect of deglycosylation on IDUA and An2-IDUA uptake in U87 cells.

FIG. 41 is a set of fluorescence confocal micrographs showing lysosomal uptake of An2 in healthy fibroblasts and MPS-I fibroblasts.

FIG. 42 is a graph showing the uptake of IDUA, An2-IDUA, Alexa-488-IDUA, and Alexa488-An2-IDUA by U87 cells.

FIG. 43 is a set of graphs showing in situ transport of IDUA and An2-IDUA across the BBB.

FIG. 44 is a schematic showing an in vitro BBB model (CELLIAL technologies) composed of a co-culture of bovine brain capillary endothelial cells with newborn rat astrocytes. This model is used to evaluate the transport across the BBB.

FIG. 45 is a graph showing evaluation of transcytosis of An2-IDUA and IDUA through brain capillary endothelial cells using the in vitro BBB model shown in FIG. 19.

FIG. 46 is a graph showing evaluation of transcytosis of An2-IDUA and IDUA through brain capillary endothelial cells using in vitro BBB model in presence of RAP or An2.

FIG. 47 is a graph showing the dose response of An2-IDUA in MPS-I patient fibroblast.

FIG. 48 is a graph showing IDUA enzymatic activity in brain homogenate of MPS-I knock-out mice. The homogenate was prepared 60 minutes after IV injection of An2-IDUA into the knock out mice.

### DETAILED DESCRIPTION

The present invention relates to compounds that include a lysosomal enzyme (e.g., IDS) and a targeting moiety (e.g., Angiopep-2) joined by a linker (e.g., a peptide bond). The targeting moiety is capable of transporting the enzyme to the lysosome and/or across the BBB. Such compounds are exemplified by Angiopep-2-IDS conjugates and fusion proteins. These proteins maintain IDS enzymatic activity both in an enzymatic assay and in a cellular model of MPS-II. Because targeting moieties such as Angiopep-2 are capable of transporting proteins across the BBB, these conjugates are expected to have not only peripheral activity, but have activity in the central nervous system (CNS). In addition, targeting moieties such as Angiopep-2 are taken up by cells by receptor mediated transport mechanism (such as LRP-1) into lysosomes. Accordingly, we believe that these targeting moieties can increase enzyme concentrations in the lysosome, thus resulting in more effective therapy, particular in tissues and organs that express the LRP-1 receptor, such as liver, kidney, and spleen.

These features overcome some of the biggest disadvantages of current therapeutic approaches because intravenous administration of IDS by itself does not treat CNS disease symptoms. In contrast to physical methods for bypassing the BBB, such intrathecal or intracranial administration, which are highly invasive and thus generally an unattractive solution to the problem of CNS delivery, the present invention allows for noninvasive brain delivery. In addition, improved transport of the therapeutic to the lysosomes may allow for reduced dosing or reduced frequency of dosing, as compared to standard enzyme replacement therapy.

### Lysosomal Storage Disorders

Lysosomal storage disorders are a group of disorders in which the metabolism of lipids, glycoproteins, or mucopolysaccharides is disrupted based on enzyme dysfunction. This dysfunction leads to cellular buildup of the substance that cannot be properly metabolized. Symptoms vary from disease to disease, but problems in the organ systems (liver, heart, lung, spleen), bones, as well as neurological problems are present in many of these diseases. Typically, these diseases are caused by rare genetic defects in the relevant enzymes. Most of these diseases are inherited in autosomal recessive fashion, but some, such as MPS-II, are X-linked recessive diseases.

### Lysosomal Enzymes

The present invention may use any lysosomal enzyme known in the art that is useful for treating a lysosomal storage disorder. The compounds of the present invention are exemplified by iduronate-2-sulfatase (IDS; also known as idursulfase). The compounds may include IDS, a fragment of IDS that retains enzymatic activity, or an IDS analog, which may include amino acid sequences substantially identical (e.g., at least 70, 80, 85, 90, 95, 96, 97, 98, or 99% identical) to the human IDS sequence and retains enzymatic activity.

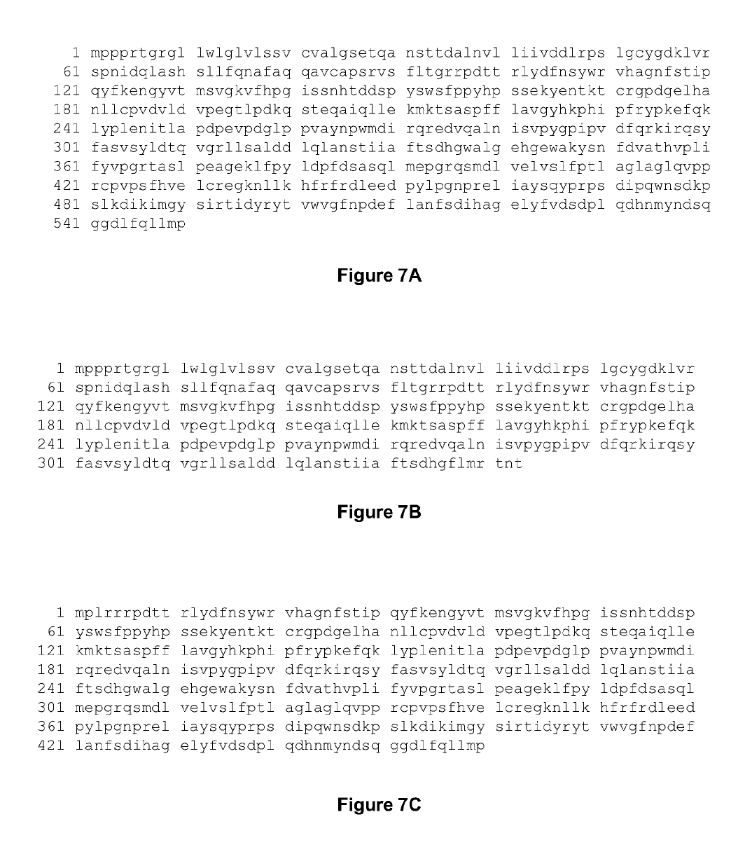

Three isoforms of IDS are known, isoforms a, b, and c. Isoform a is a 550 amino acid protein and is shown in FIG. 7A. Isoform b (FIG. 7B) is a 343 amino acid protein which has a different C-terminal region as compared to the longer Isoform a. Isoform c (FIG. 7C) has changes at the N-terminal due to the use of a downstream start codon. Any of these isoforms may be used in the compounds of the invention.

To test whether particular fragment or analog has enzymatic activity, the skilled artisan can use any appropriate assay. Assays for measuring IDS activity, for example, are known in art, including those described in Hopwood, Carbohydr. Res. 69:203-16, 1979, Bielicki et al., Biochem. J. 271:75-86, 1990, and Dean et al., Clin. Chem. 52:643-9, 2006. A similar fluorometric assay is also described below. Using any of these assays, the skilled artisan would be able to determine whether a particular IDS fragment or analog has enzymatic activity.

In certain embodiments, an enzyme fragment (e.g., an IDS fragment) is used. IDS fragments may be at least 50, 100, 150, 200, 250, 300, 350, 400, 450, or 500 amino in length. In certain embodiments, the enzyme may be modified, e.g., using any of the polypeptide modifications described herein.

### Targeting Moieties

The compounds of the invention can feature any of targeting moieties described herein, for example, any of the peptides described in Table 1 (e.g., Angiopep-1, Angiopep-2, or reversed Angiopep-2), or a fragment or analog thereof. In certain embodiments, the polypeptide may have at least 35%, 40%, 50%, 60%, 70%, 80%, 90%, 95%, 99%, or even 100% identity to a polypeptide described herein. The polypeptide may have one or more (e.g., 2, 3, 4, 5, 6, 7, 8, 9, 10, 11, 12, 13, 14, or 15) substitutions relative to one of the sequences described herein. Other modifications are described in greater detail below.

The invention also features fragments of these polypeptides (e.g., a functional fragment). In certain embodiments, the fragments are capable of efficiently being transported to or accumulating in a particular cell type (e.g., liver, eye, lung, kidney, or spleen) or are efficiently transported across the BBB. Truncations of the polypeptide may be 1, 2, 3, 4, 5, 6, 7, 8, 9, 10, 11, 12, or more amino acids from either the N-terminus of the polypeptide, the C-terminus of the polypeptide, or a combination thereof. Other fragments include sequences where internal portions of the polypeptide are deleted.

Additional polypeptides may be identified by using one of the assays or methods described herein. For example, a candidate polypeptide may be produced by conventional peptide synthesis, conjugated with paclitaxel and administered to a laboratory animal. A biologically-active polypeptide conjugate may be identified, for example, based on its ability to increase survival of an animal injected with tumor cells and treated with the conjugate as compared to a control which has not been treated with a conjugate (e.g., treated with the unconjugated agent). For example, a biologically active polypeptide may be identified based on its location in the parenchyma in an in situ cerebral perfusion assay.

Assays to determine accumulation in other tissues may be performed as well. Labelled conjugates of a polypeptide can be administered to an animal, and accumulation in different organs can be measured. For example, a polypeptide conjugated to a detectable label (e.g., a near-IR fluorescence spectroscopy label such as Cy5.5) allows live in vivo visualization. Such a polypeptide can be administered to an animal, and the presence of the polypeptide in an organ can be detected, thus allowing determination of the rate and amount of accumulation of the polypeptide in the desired organ. In other embodiments, the polypeptide can be labelled with a radioactive isotope (e.g., ¹²⁵I). The polypeptide is then administered to an animal. After a period of time, the animal is sacrificed and the organs are extracted. The amount of radioisotope in each organ can then be measured using any means known in the art. By comparing the amount of a labeled candidate polypeptide in a particular organ relative to the amount of a labeled control polypeptide, the ability of the candidate polypeptide to access and accumulate in a particular tissue can be ascertained. Appropriate negative controls include any peptide or polypeptide known not to be efficiently transported into a particular cell type (e.g., a peptide related to Angiopep that does not cross the BBB, or any other peptide).

Additional sequences are described in U.S. Pat. No. 5,807,980 (e.g., SEQ ID NO:102 herein), 5,780,265 (e.g., SEQ ID NO:103), 5,118,668 (e.g., SEQ ID NO:105). An exemplary nucleotide sequence encoding an aprotinin analog atgagaccag atttctgcct cgagccgccg tacactgggc cctgcaaagc tcgtatcatc cgttacttct acaatgcaaa ggcaggcctg tgtcagacct tcgtatacgg cggctgcaga gctaagcgta acaacttcaa atccgcggaa gactgcatgc gtacttgcgg tggtgcttag; SEQ ID NO:106; Genbank accession No. X04666). Other examples of aprotinin analogs may be found by performing a protein BLAST (Genbank: www.ncbi.nlm.nih gov/BLAST/) using the synthetic aprotinin sequence (or portion thereof) disclosed in International Application No. PCT/CA2004/000011. Exemplary aprotinin analogs are also found under accession Nos. CAA37967 (GI:58005) and 1405218C (GI:3604747).

### Modified Polypeptides

The fusion proteins, targeting moieties, and lysosomal enzymes, fragments, or analogs used in the invention may have a modified amino acid sequence. In certain embodiments, the modification does not destroy significantly a desired biological activity (e.g., ability to cross the BBB or enzymatic activity). The modification may reduce (e.g., by at least 5%, 10%, 20%, 25%, 35%, 50%, 60%, 70%, 75%, 80%, 90%, or 95%), may have no effect, or may increase (e.g., by at least 5%, 10%, 25%, 50%, 100%, 200%, 500%, or 1000%) the biological activity of the original polypeptide. The modified peptide vector or polypeptide therapeutic may have or may optimize a characteristic of a polypeptide, such as in vivo stability, bioavailability, toxicity, immunological activity, immunological identity, and conjugation properties.

Modifications include those by natural processes, such as posttranslational processing, or by chemical modification techniques known in the art. Modifications may occur anywhere in a polypeptide including the polypeptide backbone, the amino acid side chains and the amino- or carboxy-terminus. The same type of modification may be present in the same or varying degrees at several sites in a given polypeptide, and a polypeptide may contain more than one type of modification. Polypeptides may be branched as a result of ubiquitination, and they may be cyclic, with or without branching Cyclic, branched, and branched cyclic polypeptides may result from posttranslational natural processes or may be made synthetically. Other modifications include pegylation, acetylation, acylation, addition of acetomidomethyl (Acm) group, ADP-ribosylation, alkylation, amidation, biotinylation, carbamoylation, carboxyethylation, esterification, covalent attachment to fiavin, covalent attachment to a heme moiety, covalent attachment of a nucleotide or nucleotide derivative, covalent attachment of drug, covalent attachment of a marker (e.g., fluorescent or radioactive), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination.

A modified polypeptide can also include an amino acid insertion, deletion, or substitution, either conservative or non-conservative (e.g., D-amino acids, desamino acids) in the polypeptide sequence (e.g., where such changes do not substantially alter the biological activity of the polypeptide). In particular, the addition of one or more cysteine residues to the amino or carboxy terminus of any of the polypeptides of the invention can facilitate conjugation of these polypeptides by, e.g., disulfide bonding. For example, Angiopep-1 (SEQ ID NO:67), Angiopep-2 (SEQ ID NO:97), or Angiopep-7 (SEQ ID NO:112) can be modified to include a single cysteine residue at the amino-terminus (SEQ ID NOS: 71, 113, and 115, respectively) or a single cysteine residue at the carboxy-terminus (SEQ ID NOS: 72, 114, and 116, respectively). Amino acid substitutions can be conservative (i.e., wherein a residue is replaced by another of the same general type or group) or non-conservative (i.e., wherein a residue is replaced by an amino acid of another type). In addition, a non-naturally occurring amino acid can be substituted for a naturally occurring amino acid (i.e., non-naturally occurring conservative amino acid substitution or a non-naturally occurring non-conservative amino acid substitution).

Polypeptides made synthetically can include substitutions of amino acids not naturally encoded by DNA (e.g., non-naturally occurring or unnatural amino acid). Examples of non-naturally occurring amino acids include D-amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Analogs may be generated by substitutional mutagenesis and retain the biological activity of the original polypeptide. Examples of substitutions identified as “conservative substitutions” are shown in Table 2. If such substitutions result in a change not desired, then other type of substitutions, denominated “exemplary substitutions” in Table 2, or as further described herein in reference to amino acid classes, are introduced and the products screened.

Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:
(1) hydrophobic: norleucine, methionine (Met), Alanine (Ala), Valine (Val), Leucine (Leu), Isoleucine (Ile), Histidine (His), Tryptophan (Trp), Tyrosine (Tyr), Phenylalanine (Phe),
(2) neutral hydrophilic: Cysteine (Cys), Serine (Ser), Threonine (Thr)
(3) acidic/negatively charged: Aspartic acid (Asp), Glutamic acid (Glu)
(4) basic: Asparagine (Asn), Glutamine (Gln), Histidine (His), Lysine (Lys), Arginine (Arg)
(5) residues that influence chain orientation: Glycine (Gly), Proline (Pro);
(6) aromatic: Tryptophan (Trp), Tyrosine (Tyr), Phenylalanine (Phe), Histidine (His),
(7) polar: Ser, Thr, Asn, Gln
(8) basic positively charged: Arg, Lys, His, and;
(9) charged: Asp, Glu, Arg, Lys, His

Other amino acid substitutions are listed in Table 2.

TABLE 2
Amino acid substitutions

| | | Conservative |
|---|---|---|
| Original residue | Exemplary substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, norleucine | Leu |

Polypeptide Derivatives and Peptidomimetics

In addition to polypeptides consisting of naturally occurring amino acids, peptidomimetics or polypeptide analogs are also encompassed by the present invention and can form the fusion proteins, targeting moieties, or lysosomal enzymes, enzyme fragments, or enzyme analogs used in the compounds of the invention. Polypeptide analogs are commonly used in the pharmaceutical industry as non-peptide drugs with properties analogous to those of the template polypeptide. The non-peptide compounds are termed “peptide mimetics” or peptidomimetics (Fauchere et al., Infect. Immun. 54:283-287,1986 and Evans et al., J. Med. Chem. 30:1229-1239, 1987). Peptide mimetics that are structurally related to therapeutically useful peptides or polypeptides may be used to produce an equivalent or enhanced therapeutic or prophylactic effect. Generally, peptidomimetics are structurally similar to the paradigm polypeptide (i.e., a polypeptide that has a biological or pharmacological activity) such as naturally-occurring receptor-binding polypeptides, but have one or more peptide linkages optionally replaced by linkages such as —CH₂NH—, —CH₂S—, —CH₂—CH₂—, —CH═CH— (cis and trans), —CH₂SO—, —CH(OH)CH₂—, —COCH₂— etc., by methods well known in the art (Spatola, Peptide Backbone Modifications, Vega Data, 1:267, 1983; Spatola et al., Life Sci. 38:1243-1249, 1986; Hudson et al., Int. J. Pept. Res. 14:177-185, 1979; and Weinstein, 1983, Chemistry and Biochemistry, of Amino Acids, Peptides and Proteins, Weinstein eds, Marcel Dekker, New York). Such polypeptide mimetics may have significant advantages over naturally occurring polypeptides including more economical production, greater chemical stability, enhanced pharmacological properties (e.g., half-life, absorption, potency, efficiency), reduced antigenicity, and others.

While the targeting moieties described herein may efficiently cross the BBB or target particular cell types (e.g., those described herein), their effectiveness may be reduced by the presence of proteases. Likewise, the effectiveness of the lysosomal enzymes, enzyme fragments, or enzyme analogs used in the compounds of the invention may be similarly reduced. Serum proteases have specific substrate requirements, including L-amino acids and peptide bonds for cleavage. Furthermore, exopeptidases, which represent the most prominent component of the protease activity in serum, usually act on the first peptide bond of the polypeptide and require a free N-terminus (Powell et al., Pharm. Res. 10:1268-1273, 1993). In light of this, it is often advantageous to use modified versions of polypeptides. The modified polypeptides retain the structural characteristics of the original L-amino acid polypeptides, but advantageously are not readily susceptible to cleavage by protease and/or exopeptidases.

Systematic substitution of one or more amino acids of a consensus sequence with D-amino acid of the same type (e.g., an enantiomer; D-lysine in place of L-lysine) may be used to generate more stable polypeptides. Thus, a polypeptide derivative or peptidomimetic as described herein may be all L-, all D-, or mixed D, L polypeptides. The presence of an N-terminal or C-terminal D-amino acid increases the in vivo stability of a polypeptide because peptidases cannot utilize a D-amino acid as a substrate (Powell et al., Pharm. Res. 10:1268-1273, 1993). Reverse-D polypeptides are polypeptides containing D-amino acids, arranged in a reverse sequence relative to a polypeptide containing L-amino acids. Thus, the C-terminal residue of an L-amino acid polypeptide becomes N-terminal for the D-amino acid polypeptide, and so forth. Reverse D-polypeptides retain the same tertiary conformation and therefore the same activity, as the L-amino acid polypeptides, but are more stable to enzymatic degradation in vitro and in vivo, and thus have greater therapeutic efficacy than the original polypeptide (Brady and Dodson, Nature 368:692-693, 1994 and Jameson et al., Nature 368:744-746, 1994). In addition to reverse-D-polypeptides, constrained polypeptides comprising a consensus sequence or a substantially identical consensus sequence variation may be generated by methods well known in the art (Rizo et al., Ann. Rev. Biochem. 61:387-418, 1992). For example, constrained polypeptides may be generated by adding cysteine residues capable of forming disulfide bridges and, thereby, resulting in a cyclic polypeptide. Cyclic polypeptides have no free N- or C-termini. Accordingly, they are not susceptible to proteolysis by exopeptidases, although they are, of course, susceptible to endopeptidases, which do not cleave at polypeptide termini. The amino acid sequences of the polypeptides with N-terminal or C-terminal D-amino acids and of the cyclic polypeptides are usually identical to the sequences of the polypeptides to which they correspond, except for the presence of N-terminal or C-terminal D-amino acid residue, or their circular structure, respectively.

A cyclic derivative containing an intramolecular disulfide bond may be prepared by conventional solid phase synthesis while incorporating suitable S-protected cysteine or homocysteine residues at the positions selected for cyclization such as the amino and carboxy termini (Sah et al., J. Pharm. Pharmacol. 48:197, 1996). Following completion of the chain assembly, cyclization can be performed either (1) by selective removal of the S-protecting group with a consequent on-support oxidation of the corresponding two free SH-functions, to form a S—S bonds, followed by conventional removal of the product from the support and appropriate purification procedure or (2) by removal of the polypeptide from the support along with complete side chain de-protection, followed by oxidation of the free SH-functions in highly dilute aqueous solution.

The cyclic derivative containing an intramolecular amide bond may be prepared by conventional solid phase synthesis while incorporating suitable amino and carboxyl side chain protected amino acid derivatives, at the position selected for cyclization. The cyclic derivatives containing intramolecular —S-alkyl bonds can be prepared by conventional solid phase chemistry while incorporating an amino acid residue with a suitable amino-protected side chain, and a suitable S-protected cysteine or homocysteine residue at the position selected for cyclization.

Another effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al., Pharm. Res. 10:1268-1273, 1993). Other chemical modifications which enhance serum stability include, but are not limited to, the addition of an N-terminal alkyl group, consisting of a lower alkyl of from one to twenty carbons, such as an acetyl group, and/or the addition of a C-terminal amide or substituted amide group. In particular, the present invention includes modified polypeptides consisting of polypeptides bearing an N-terminal acetyl group and/or a C-terminal amide group.

Also included by the present invention are other types of polypeptide derivatives containing additional chemical moieties not normally part of the polypeptide, provided that the derivative retains the desired functional activity of the polypeptide. Examples of such derivatives include (1) N-acyl derivatives of the amino terminal or of another free amino group, wherein the acyl group may be an alkanoyl group (e.g., acetyl, hexanoyl, octanoyl) an aroyl group (e.g., benzoyl) or a blocking group such as F-moc (fluorenylmethyl-O—CO—); (2) esters of the carboxy terminal or of another free carboxy or hydroxyl group; (3) amide of the carboxy-terminal or of another free carboxyl group produced by reaction with ammonia or with a suitable amine; (4) phosphorylated derivatives; (5) derivatives conjugated to an antibody or other biological ligand and other types of derivatives.

Longer polypeptide sequences which result from the addition of additional amino acid residues to the polypeptides described herein are also encompassed in the present invention. Such longer polypeptide sequences can be expected to have the same biological activity and specificity (e.g., cell tropism) as the polypeptides described above. While polypeptides having a substantial number of additional amino acids are not excluded, it is recognized that some large polypeptides may assume a configuration that masks the effective sequence, thereby preventing binding to a target (e.g., a member of the LRP receptor family). These derivatives could act as competitive antagonists. Thus, while the present invention encompasses polypeptides or derivatives of the polypeptides described herein having an extension, desirably the extension does not destroy the cell targeting activity or enzymatic activity of the compound.

Other derivatives included in the present invention are dual polypeptides consisting of two of the same, or two different polypeptides, as described herein, covalently linked to one another either directly or through a spacer, such as by a short stretch of alanine residues or by a putative site for proteolysis (e.g., by cathepsin, see e.g., U.S. Pat. No. 5,126,249 and European Patent No. 495 049). Multimers of the polypeptides described herein consist of a polymer of molecules formed from the same or different polypeptides or derivatives thereof.

The present invention also encompasses polypeptide derivatives that are chimeric or fusion proteins containing a polypeptide described herein, or fragment thereof, linked at its amino- or carboxy-terminal end, or both, to an amino acid sequence of a different protein. Such a chimeric or fusion protein may be produced by recombinant expression of a nucleic acid encoding the protein. For example, a chimeric or fusion protein may contain at least 6 amino acids shared with one of the described polypeptides which desirably results in a chimeric or fusion protein that has an equivalent or greater functional activity.

Assays to Identify Peptidomimetics

As described above, non-peptidyl compounds generated to replicate the backbone geometry and pharmacophore display (peptidomimetics) of the polypeptides described herein often possess attributes of greater metabolic stability, higher potency, longer duration of action, and better bioavailability.

Peptidomimetics compounds can be obtained using any of the numerous approaches in combinatorial library methods known in the art, including biological libraries, spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer, or small molecule libraries of compounds (Lam, Anticancer Drug Des. 12:145, 1997). Examples of methods for the synthesis of molecular libraries can be found in the art, for example, in: DeWitt et al. (Proc. Natl. Acad. Sci. USA 90:6909, 1993); Erb et al. (Proc. Natl. Acad. Sci. USA 91:11422, 1994); Zuckermann et al. (J. Med. Chem. 37:2678, 1994); Cho et al. (Science 261:1303, 1993); Carell et al. (Angew. Chem, Int. Ed. Engl. 33:2059, 1994 and ibid 2061); and in Gallop et al. (Med. Chem. 37:1233, 1994). Libraries of compounds may be presented in solution (e.g., Houghten, Biotechniques 13:412-421, 1992) or on beads (Lam, Nature 354:82-84, 1991), chips (Fodor, Nature 364:555-556, 1993), bacteria or spores (U.S. Pat. No. 5,223,409), plasmids (Cull et al., Proc. Natl. Acad. Sci. USA 89:1865-1869, 1992) or on phage (Scott and Smith, Science 249:386-390, 1990), or luciferase, and the enzymatic label detected by determination of conversion of an appropriate substrate to product.

Once a polypeptide as described herein is identified, it can be isolated and purified by any number of standard methods including, but not limited to, differential solubility (e.g., precipitation), centrifugation, chromatography (e.g., affinity, ion exchange, and size exclusion), or by any other standard techniques used for the purification of peptides, peptidomimetics, or proteins. The functional properties of an identified polypeptide of interest may be evaluated using any functional assay known in the art. Desirably, assays for evaluating downstream receptor function in intracellular signaling are used (e.g., cell proliferation).

For example, the peptidomimetics compounds of the present invention may be obtained using the following three-phase process: (1) scanning the polypeptides described herein to identify regions of secondary structure necessary for targeting the particular cell types described herein; (2) using conformationally constrained dipeptide surrogates to refine the backbone geometry and provide organic platforms corresponding to these surrogates; and (3) using the best organic platforms to display organic pharmocophores in libraries of candidates designed to mimic the desired activity of the native polypeptide. In more detail the three phases are as follows. In phase 1, the lead candidate polypeptides are scanned and their structure abridged to identify the requirements for their activity. A series of polypeptide analogs of the original are synthesized. In phase 2, the best polypeptide analogs are investigated using the conformationally constrained dipeptide surrogates. Indolizidin-2-one, indolizidin-9-one and quinolizidinone amino acids (I²aa, I⁹aa and Qaa respectively) are used as platforms for studying backbone geometry of the best peptide candidates. These and related platforms (reviewed in Halab et al., Biopolymers 55:101-122, 2000 and Hanessian et al., Tetrahedron 53:12789-12854, 1997) may be introduced at specific regions of the polypeptide to orient the pharmacophores in different directions. Biological evaluation of these analogs identifies improved lead polypeptides that mimic the geometric requirements for activity. In phase 3, the platforms from the most active lead polypeptides are used to display organic surrogates of the pharmacophores responsible for activity of the native peptide. The pharmacophores and scaffolds are combined in a parallel synthesis format. Derivation of polypeptides and the above phases can be accomplished by other means using methods known in the art.

Structure function relationships determined from the polypeptides, polypeptide derivatives, peptidomimetics or other small molecules described herein may be used to refine and prepare analogous molecular structures having similar or better properties. Accordingly, the compounds of the present invention also include molecules that share the structure, polarity, charge characteristics and side chain properties of the polypeptides described herein.

In summary, based on the disclosure herein, those skilled in the art can develop peptides and peptidomimetics screening assays which are useful for identifying compounds for targeting an agent to particular cell types (e.g., those described herein). The assays of this invention may be developed for low-throughput, high-throughput, or ultra-high throughput screening formats. Assays of the present invention include assays amenable to automation.

### Linkers

The lysosomal enzyme (e.g., IDS), enzyme fragment, or enzyme analog may be bound to the targeting moiety either directly (e.g., through a covalent bond such as a peptide bond) or may be bound through a linker. Linkers include chemical linking agents (e.g., cleavable linkers) and peptides.

In some embodiments, the linker is a chemical linking agent. The lysosomal enzyme (e.g., IDS), enzyme fragment, or enzyme analog and targeting moiety may be conjugated through sulfhydryl groups, amino groups (amines), and/or carbohydrates or any appropriate reactive group. Homobifunctional and heterobifunctional cross-linkers (conjugation agents) are available from many commercial sources. Regions available for cross-linking may be found on the polypeptides of the present invention. The cross-linker may comprise a flexible arm, e.g., 2, 3, 4, 5, 6, 7, 8, 9, 10, 11, 12, 13, 14, or 15 carbon atoms. Exemplary cross-linkers include BS3 ([Bis(sulfosuccinimidyl)suberate]; BS3 is a homobifunctional N-hydroxysuccinimide ester that targets accessible primary amines), NHS/EDC (N-hydroxysuccinimide and N-ethyl-′(dimethylaminopropyl)carbodimide; NHS/EDC allows for the conjugation of primary amine groups with carboxyl groups), sulfo-EMCS ([N-e-Maleimidocaproic acid]hydrazide; sulfo-EMCS are heterobifunctional reactive groups (maleimide and NHS-ester) that are reactive toward sulfhydryl and amino groups), hydrazide (most proteins contain exposed carbohydrates and hydrazide is a useful reagent for linking carboxyl groups to primary amines), and SATA (N-succinimidyl-S-acetylthioacetate; SATA is reactive towards amines and adds protected sulfhydryls groups).

To form covalent bonds, one can use as a chemically reactive group a wide variety of active carboxyl groups (e.g., esters) where the hydroxyl moiety is physiologically acceptable at the levels required to modify the peptide. Particular agents include N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), maleimido propionic acid (MPA) maleimido hexanoic acid (MHA), and maleimido undecanoic acid (MUA).

Primary amines are the principal targets for NHS esters. Accessible a-amine groups present on the N-termini of proteins and the ε-amine of lysine react with NHS esters. An amide bond is formed when the NHS ester conjugation reaction reacts with primary amines releasing N-hydroxysuccinimide. These succinimide containing reactive groups are herein referred to as succinimidyl groups. In certain embodiments of the invention, the functional group on the protein will be a thiol group and the chemically reactive group will be a maleimido-containing group such as gamma-maleimide-butrylamide (GMBA or MPA). Such maleimide containing groups are referred to herein as maleido groups.

The maleimido group is most selective for sulfhydryl groups on peptides when the pH of the reaction mixture is 6.5-7.4. At pH 7.0, the rate of reaction of maleimido groups with sulfhydryls (e.g., thiol groups on proteins such as serum albumin or IgG) is 1000-fold faster than with amines. Thus, a stable thioether linkage between the maleimido group and the sulfhydryl can be formed.

In other embodiments, the linker includes at least one amino acid (e.g., a peptide of at least 2, 3, 4, 5, 6, 7, 10, 15, 20, 25, 40, or 50 amino acids). In certain embodiments, the linker is a single amino acid (e.g., any naturally occurring amino acid such as Cys). In other embodiments, a glycine-rich peptide such as a peptide having the sequence [Gly-Gly-Gly-Gly-Ser]ₙ where n is 1, 2, 3, 4, 5 or 6 is used, as described in U.S. Pat. No. 7,271,149. In other embodiments, a serine-rich peptide linker is used, as described in U.S. Pat. No. 5,525,491. Serine rich peptide linkers include those of the formula [X-X-X-X-Gly]_{y}, where up to two of the X are Thr, and the remaining X are Ser, and y is 1 to 5 (e.g., Ser-Ser-Ser-Ser-Gly, where y is greater than 1). In some cases, the linker is a single amino acid (e.g., any amino acid, such as Gly or Cys). Other linkers include rigid linkers (e.g., PAPAP and (PT)ₙP, where n is 2, 3, 4, 5, 6, or 7) and α-helical linkers (e.g., A(EAAAK)ₙA, where n is 1, 2, 3, 4, or 5).

Examples of suitable linkers are succinic acid, Lys, Glu, and Asp, or a dipeptide such as Gly-Lys. When the linker is succinic acid, one carboxyl group thereof may form an amide bond with an amino group of the amino acid residue, and the other carboxyl group thereof may, for example, form an amide bond with an amino group of the peptide or substituent. When the linker is Lys, Glu, or Asp, the carboxyl group thereof may form an amide bond with an amino group of the amino acid residue, and the amino group thereof may, for example, form an amide bond with a carboxyl group of the substituent. When Lys is used as the linker, a further linker may be inserted between the ε-amino group of Lys and the substituent. In one particular embodiment, the further linker is succinic acid which, e.g., forms an amide bond with the ε-amino group of Lys and with an amino group present in the substituent. In one embodiment, the further linker is Glu or Asp (e.g., which forms an amide bond with the ε-amino group of Lys and another amide bond with a carboxyl group present in the substituent), that is, the substituent is an N^{ε}-acylated lysine residue.

### Click-Chemistry Linkers

In particular embodiments, the linker is formed by the reaction between a click-chemistry reaction pair. By click-chemistry reaction pair is meant a pair of reactive groups that participates in a modular reaction with high yield and a high thermodynamic gain, thus producing a click-chemistry linker. In this embodiment, one of the reactive groups is attached to the enzyme moiety and the other reactive group is attached to the polypeptide. Exemplary reactions and click-chemistry pairs include a Huisgen 1,3-dipolar cycloaddition reaction between an alkynyl group and an azido group to form a triazole-containing linker; a Diels-Alder reaction between a diene having a 4π electron system (e.g., an optionally substituted 1,3-unsaturated compound, such as optionally substituted 1,3-butadiene, 1-methoxy-3-trimethylsilyloxy-1,3-butadiene, cyclopentadiene, cyclohexadiene, or furan) and a dienophile or heterodienophile having a 2π electron system (e.g., an optionally substituted alkenyl group or an optionally substituted alkynyl group); a ring opening reaction with a nucleophile and a strained heterocyclyl electrophile; a splint ligation reaction with a phosphorothioate group and an iodo group; and a reductive amination reaction with an aldehyde group and an amino group (Kolb et al., Angew. Chem. Int. Ed., 40:2004-2021 (2001); Van der Eycken et al., QSAR Comb. Sci., 26:1115-1326 (2007)).

In particular embodiments of the invention, the polypeptide is linked to the enzyme moiety by means of a triazole-containing linker formed by the reaction between a alkynyl group and an azido group click-chemistry pair. In such cases, the azido group may be attached to the polypeptide and the alkynyl group may be attached to the enzyme moiety. Alternatively, the azido group may be attached to the enzyme moiety and the alkynyl group may be attached to the polypeptide. In certain embodiments, the reaction between an azido group and the alkynyl group is uncatalyzed, and in other embodiments the reaction is catalyzed by a copper(I) catalyst (e.g., copper(I) iodide), a copper(II) catalyst in the presence of a reducing agent (e.g., copper(II) sulfate or copper(II) acetate with sodium ascorbate), or a ruthenium-containing catalyst (e.g., Cp*RuCl(PPh₃)₂ or Cp*RuCl(COD)).

Exemplary linkers include monofluorocyclooctyne (MFCO), difluorocyclooctyne (DFCO), cyclooctyne (OCT), dibenzocyclooctyne (DIBO), biarylazacyclooctyne (BARAC), difluorobenzocyclooctyne (DIFBO), and bicyclo[6.1.0]nonyne (BCN).

### Treatment of Lysosomal Storage Disorders

The present invention also features methods for treatment of lysosomal storage disorders such as MPS-II. MPS-II is characterized by cellular accumulation of glycosaminoglycans (GAG) which results from the inability of the individual to break down these products.

In certain embodiments, treatment is performed on a subject who has been diagnosed with a mutation in the IDS gene, but does not yet have disease symptoms (e.g., an infant or subject under the age of 2). In other embodiments, treatment is performed on an individual who has at least one MPS-II symptom (e.g., any of those described herein).

MPS-II is generally classified into two general groups, severe disease and attenuated disease. The present invention can involve treatment of subjects with either type of disease. Severe disease is characterized by CNS involvement. In severe disease the cognitive decline, coupled with airway and cardiac disease, usually results in death before adulthood. The attenuated form of the disease general involves only minimal or no CNS involvement. In both severe and attenuated disease, the non-CNS symptoms can be as severe as those with the “severe” form.

Initial MPS-II symptoms begin to manifest themselves from about 18 months to about four years of age and include abdominal hernias, ear infections, runny noses, and colds. Symptoms include coarseness of facial features (e.g., prominent forehead, nose with a flattened bridge, and an enlarged tongue), large head (macrocephaly), enlarged abdomen, including enlarged liver (heptaomegaly) and enlarged spleen (slenomegaly), and hearing loss. The methods of the invention may involve treatment of subjects having any of the symptoms described herein. MPS-II also results in joint abnormalities, related to thickening of bones.

Treatment may be performed in a subject of any age, starting from infancy to adulthood. Subjects may begin treatment at birth, six months, or 1, 2, 3, 4, 5, 6, 7, 8, 9, 10, 11, 12, 13, 15, or 18 years of age.

### Administration and Dosage

The present invention also features pharmaceutical compositions that contain a therapeutically effective amount of a compound of the invention. The composition can be formulated for use in a variety of drug delivery systems. One or more physiologically acceptable excipients or carriers can also be included in the composition for proper formulation. Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The pharmaceutical compositions are intended for parenteral, intranasal, topical, oral, or local administration, such as by a transdermal means, for prophylactic and/or therapeutic treatment. The pharmaceutical compositions can be administered parenterally (e.g., by intravenous, intramuscular, or subcutaneous injection), or by oral ingestion, or by topical application or intraarticular injection at areas affected by the vascular or cancer condition. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Sustained release administration is also specifically included in the invention, by such means as depot injections or erodible implants or components. Thus, the invention provides compositions for parenteral administration that include the above mention agents dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. The invention also provides compositions for oral delivery, which may contain inert ingredients such as binders or fillers for the formulation of a tablet, a capsule, and the like. Furthermore, this invention provides compositions for local administration, which may contain inert ingredients such as solvents or emulsifiers for the formulation of a cream, an ointment, and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The compositions containing an effective amount can be administered for prophylactic or therapeutic treatments. In prophylactic applications, compositions can be administered to a subject diagnosed as having mutation associated with a lysosomal storage disorder (e.g., a mutation in the IDS gene). Compositions of the invention can be administered to the subject (e.g., a human) in an amount sufficient to delay, reduce, or preferably prevent the onset of the disorder. In therapeutic applications, compositions are administered to a subject (e.g., a human) already suffering from a lysosomal storage disorder (e.g., MPS-II) in an amount sufficient to cure or at least partially arrest the symptoms of the disorder and its complications. An amount adequate to accomplish this purpose is defined as a “therapeutically effective amount,” an amount of a compound sufficient to substantially improve at least one symptom associated with the disease or a medical condition. For example, in the treatment of a lysosomal storage disease, an agent or compound that decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual.

Amounts effective for this use may depend on the severity of the disease or condition and the weight and general state of the subject. Idursulfase is recommended for weekly intravenous administration of 0.5 mg/kg. A compound of the invention may, for example, be administered at an equivalent dosage (i.e., accounting for the additional molecular weight of the fusion protein vs. idursulfase) and frequency. The compound may be administered at an iduronase equivalent dose, e.g., 0.01, 0.05, 0.1, 0.5, 0.1, 0.2, 0.3, 0.4, 0.5, 0.75, 1.0, 1.25, 1.5, 2.0, 2.5, 3.0, 4.0, or 5 mg/kg weekly, twice weekly, every other day, daily, or twice daily. The therapeutically effective amount of the compositions of the invention and used in the methods of this invention applied to mammals (e.g., humans) can be determined by the ordinarily-skilled artisan with consideration of individual differences in age, weight, and the condition of the mammal. Because certain compounds of the invention exhibit an enhanced ability to cross the BBB and to enter lysosomes, the dosage of the compounds of the invention can be lower than (e.g., less than or equal to about 90%, 75%, 50%, 40%, 30%, 20%, 15%, 12%, 10%, 8%, 7%, 6%, 5%, 4%, 3%, 2%, 1%, 0.5%, or 0.1% of) the equivalent dose of required for a therapeutic effect of the unconjugated agent. The agents of the invention are administered to a subject (e.g. a mammal, such as a human) in an effective amount, which is an amount that produces a desirable result in a treated subject (e.g., reduction of GAG accumulation). Therapeutically effective amounts can also be determined empirically by those of skill in the art.

Single or multiple administrations of the compositions of the invention including an effective amount can be carried out with dose levels and pattern being selected by the treating physician. The dose and administration schedule can be determined and adjusted based on the severity of the disease or condition in the subject, which may be monitored throughout the course of treatment according to the methods commonly practiced by clinicians or those described herein.

The compounds of the present invention may be used in combination with either conventional methods of treatment or therapy or may be used separately from conventional methods of treatment or therapy.

When the compounds of this invention are administered in combination therapies with other agents, they may be administered sequentially or concurrently to an individual. Alternatively, pharmaceutical compositions according to the present invention may be comprised of a combination of a compound of the present invention in association with a pharmaceutically acceptable excipient, as described herein, and another therapeutic or prophylactic agent known in the art.

The following examples are intended to illustrate, rather than limit, the invention.

### Example 1

### Design of IDS-Angiopep-2 Fusion Proteins

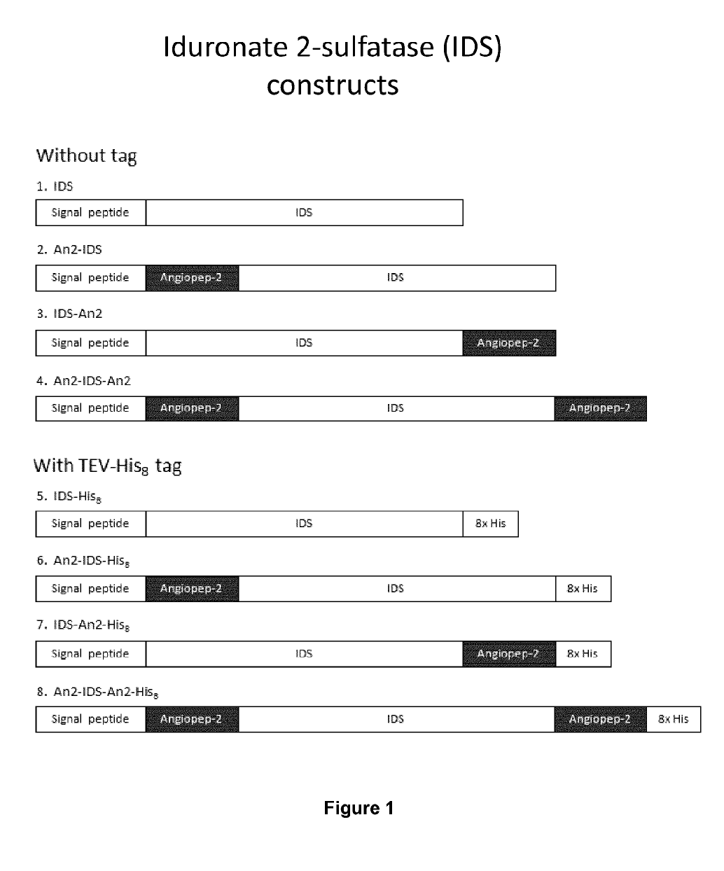

A series of IDS-Angiopep-2 constructs were designed. The IDS cDNA was obtained from Origene (Cat. No. RC219187). Three basic configurations were used: an N-terminal fusion (An2-IDS and An2-IDS-His), a C-terminal fusion (IDS-An2 and IDS-An2-His), and an N- and C-terminal fusion (An2-IDS-An2 and An2-IDS-An2-His), both with and without an 8×His tag (FIG. 1). A control without Angiopep-2 was also generated (IDS and IDS-His).

### Example 2

### Expression and Activity of Recombinant hIDS Proteins in CHO—S Cells

These constructs were then expressed in CHO—S cells grown in suspension. IDS constructs were expressed by transient transfection in FreeStyle CHO—S cells (Invitrogen), using linear 25 kDa polyethyleneimine (PEI, Polyscience) as the transfection reagent. In one example, DNA (1 mg) was mixed with 70 ml FreeStyle CHO Expression medium (Invitrogen) and incubated at room temperature for 15 min PEI (2 mg) was separately incubated in 70 ml medium for 15 minutes, and then DNA and PEI solutions were mixed and further incubated for 15 min. The DNA/PEI complex mixture was added to 360 ml of medium containing 1×10⁹ CHO—S cells. After a four-hour incubation at 37° C., 8% CO₂ with moderate agitation, 500 ml of warm medium was added. CHO—S cells were further incubated for 5 days in the same conditions before harvesting.

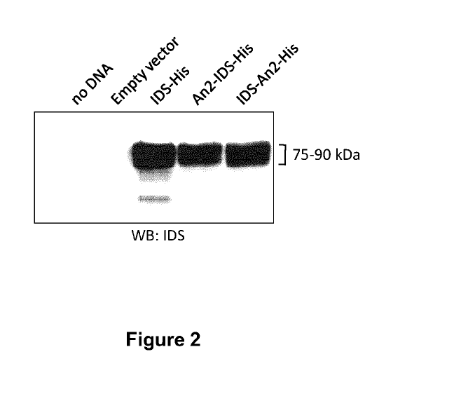

To determine if the cells were expressing and secreting IDS or an IDS fusion protein, a western blot using an anti-IDS antibody was performed on the culture medium. As can be seen in FIG. 2, expression levels of IDS-His, An2-IDS-His and IDS-An2-His were similar. Thus, the cells were able to express these proteins.

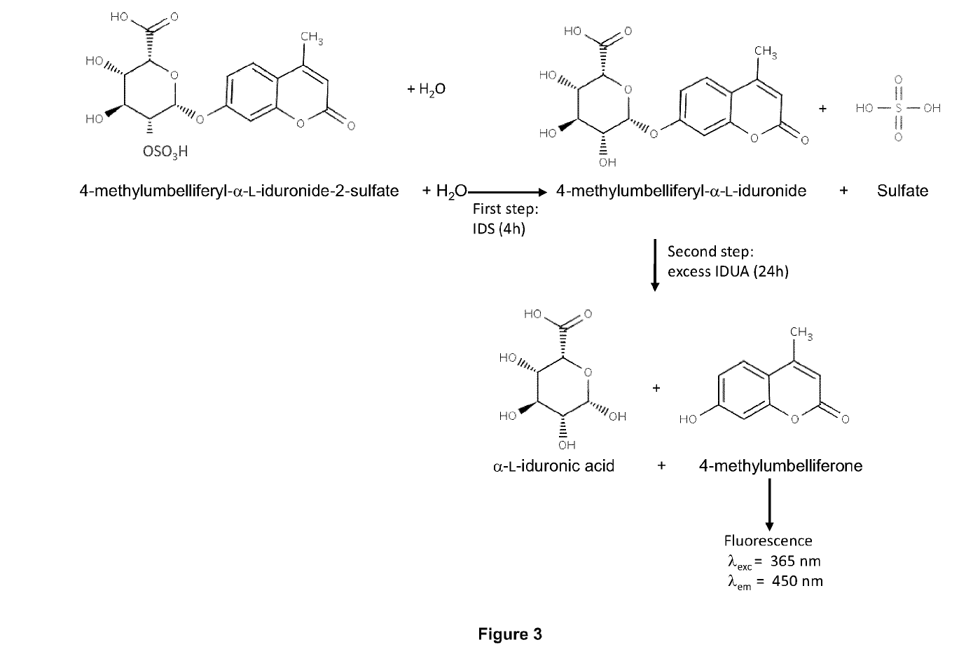

We also characterized IDS activity in the media. This assay was performed using a two-step enzymatic assay (FIG. 3). This assay involves treating 4-methylumbelliferyl-a-L-iduronide-2-sulfate in water with IDS for 4 hours to generate 4-methylumbelliferyl-a-L-iduronide and sulfate. In a second step, these products were treated with excess a-L-iduronidase (IDUA) for 24 hours to generate a-L-iduronic acid and 4-methylumbelliferone. Activity was determined by measuring fluorescence of 4-methylumbelliferone (365 nm excitation; 450 nm emission).

In one particular example, this assay was performed as follows. Ten μl of media from CHO—S transfected cells was mixed with 20 μl of 1.25 mM 4-methylumbelliferyl-alpha-L-iduronide-2-sulphate (IDS substrate from Moscerdam Substrates) in acetate buffer, pH 5.0, and incubated for 4 h at 37° C. The second step of the assay was then initiated by adding 20 μl 0.2 M Na₂HPO₄/0.1 M citric acid buffer, pH 4.5 and 10 μl lysosomal enzymes purified from bovine testis (LEBT). After 24 h at 37° C., the reaction was stopped with 200 μl 0.5 M NaHCO3/Na₂CO3 buffer, pH 10.7, containing 0.025% Triton X-100. Activity was determined by measuring fluorescence of 4-methylumbelliferone (365 nm excitation; 450 nm emission).

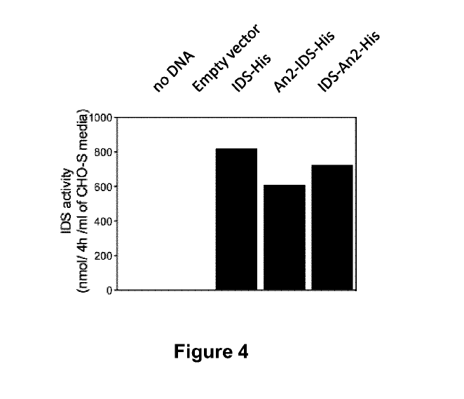

Measurements of IDS activity in the CHO—S cells grown in suspension is shown in FIG. 4, and all three proteins (IDS-His, An2-IDS-His, and IDS-AN2-His) were shown to have IDS activity.

### Example 3

### Characterization and Optimization of Expression

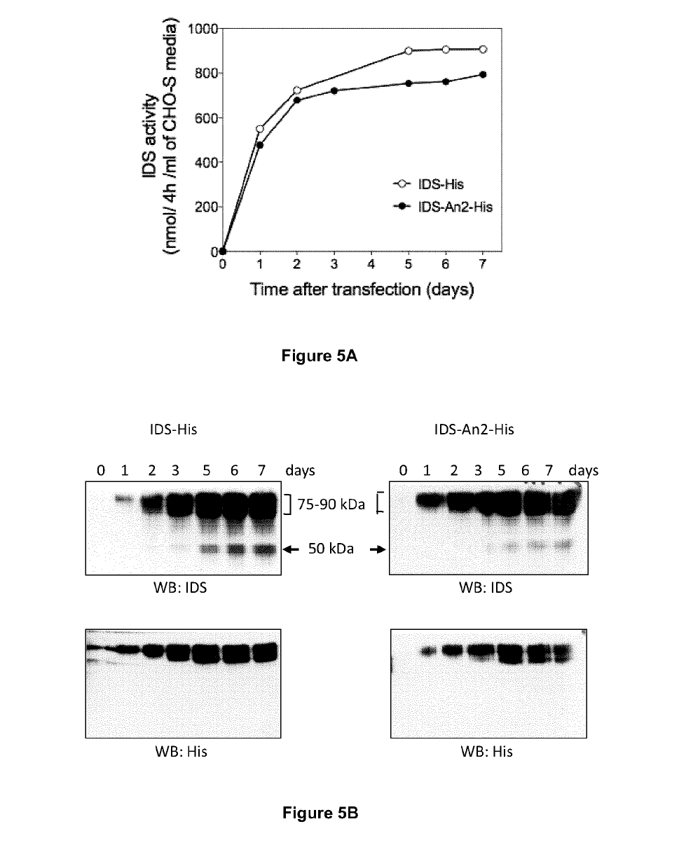

To further characterize expression, time course evaluation of IDS expression and activity in CHO—S cells grown in suspension was measured for the IDS-His and IDS-An2-His fusion proteins as shown in FIG. 5A and FIG. 5B. From these data, maximal IDS expression and activity was observed five days after transfection. No recapture of IDS-An2-His by CHO—S cells was observed in these experiments.

To further optimize transfection conditions, transfection was performed using two different numbers of cells (1.25×10⁷ cells or 2.5×10⁷ cells). Three different ratios of DNA to polyethylenimine (PEI) were used (1:1, 1:2, 1:3, and 1:4).

From these experiments, the best results were obtained using a 1:2 DNA:PEI ratio, as shown by the IDS activity (FIG. 5A) and by expression analysis (FIG. 5B).

### Example 4

### IDS Activity in MPS-II Fibroblasts

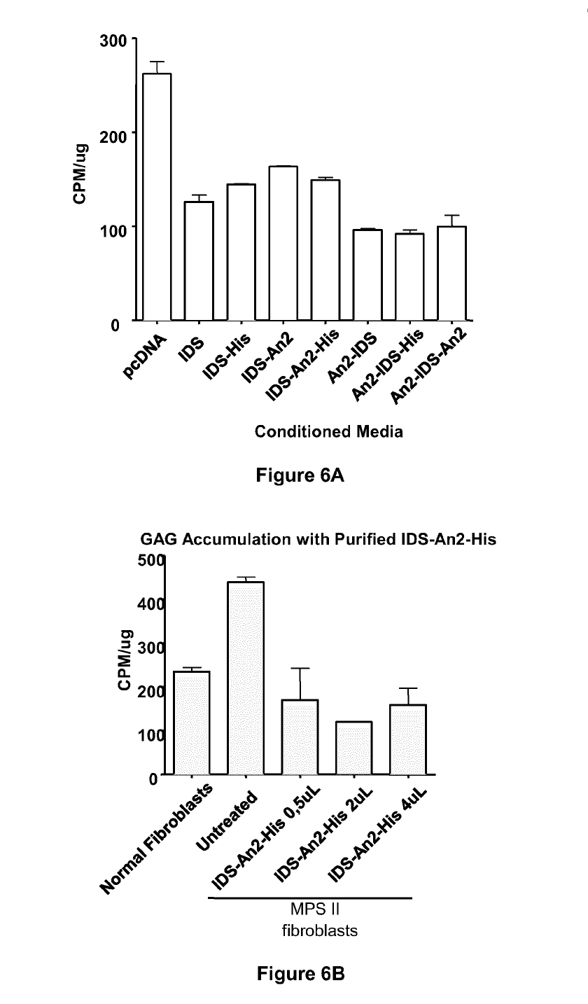

To determine whether, the expressed proteins are capable of reducing GAG accumulation in cells, fibroblasts taken from an MPS-II patient were used. In a first set of experiments, cell culture medium from the above-described CHO—S cells transfected with various IDS and IDS fusion proteins was incubated with the fibroblasts. GAG accumulation was measured based on the presence of 35S-GAG. As shown in FIG. 6A, reduction of GAG using the fusion proteins was similar to that of IDS itself.

These assays were performed as follows. MPS II (Coriell institute, GM00298), or healthy human fibroblasts (GM05659) were plated in 6-well dishes at 250,000 cells/well in DMEM with 10% fetal bovine serum (FBS) and grown at 37° C. under 5% CO₂. After 4 days, cells were washed once with PBS and once with low sulfate F-12 medium (Invitrogen, catalog #11765-054). One ml of low sulfate F-12 medium containing 10% dialyzed FBS (Sigma, catalog # F0392) and 10 μCi ³⁵S-sodium sulfate was added to the cells in the absence or presence of recombinant IDS proteins. Fibroblasts were incubated at 37° C. under 5% CO₂. After 48 h, medium was removed and cells were washed 5 times with PBS. Cells were lysed in 0.4 ml/well of 1 N NaOH and heated at 60° C. for 60 min to solubilize proteins. An aliquot was removed for μBCA protein assay. Radioactivity was counted with a liquid scintillation counter. The data are expressed as ³⁵S CPM per μg protein.

Even more promising results were obtained with purified IDS-An2-his which was able to decrease the GAG-accumulation to normal control value measured in normal human fibroblasts (FIG. 6B). These results indicate that our purified fusion protein is active. In sum, these data with MPS-II fibroblasts indicate that the fusion proteins are active and that they reach the lysosomes where they can cleave the glycoaminoglycans.

Finally, western blots show that LRP-1 is expressed at the same levels in normal and MPS-II fibroblasts (data not shown).

### Example 5

### Click Chemistry Linkers

In one example, the targeting moiety is joined to the lysosomal enzyme through a click chemistry linker. An example of this chemistry is shown below.

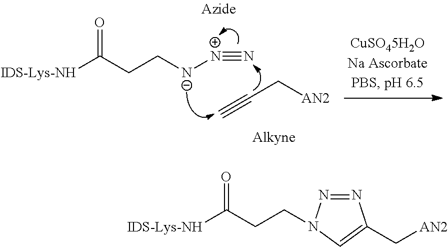

This approach is advantageous in that it is very selective because the reaction only occurs between the azide and alkyne components. The reaction also takes place in aqueous solution and is biocompatible and can be performed in living cells. In addition, the reaction is rapid and quantitative, allowing preparation of nanomoles of conjugates in dilute solutions. Finally, because the reaction is pH-insensitive, it can be performed anywhere from pH 4 to 11. Specific click chemistry linkers used in the invention are discussed in Examples 8 and 9.

### Example 6

### SATA Chemical Linkage

In another example the targeting moiety is joined to the lysosomal enzyme through an SATA chemical linker. An exemplary scheme for generating such a conjugate is shown below.

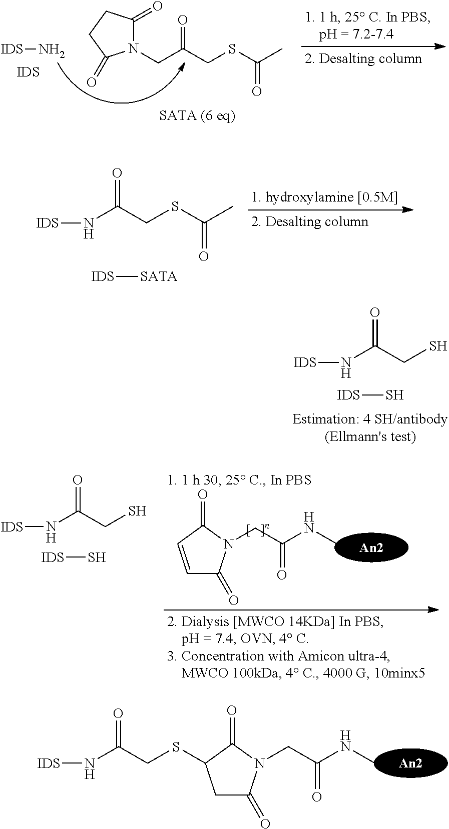

### Example 7

### Other Chemical Conjugation Strategies

In another example, chemical conjugation is achieved through a hydrazide linker. An exemplary scheme for generation of such a conjugate is as follows.

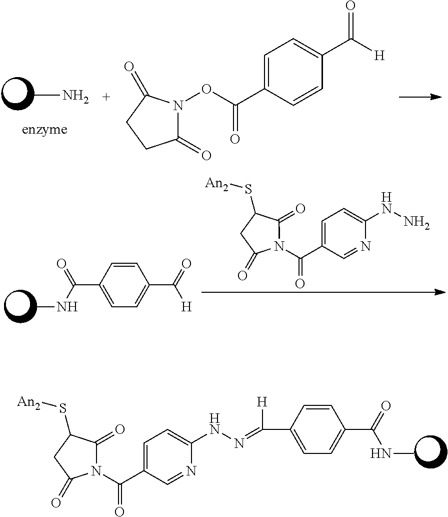

In another example, chemical conjugation is achieved using a periodate-oxidated enzyme with a hydrazide derivative through a sugar moiety (e.g., a glycosylation site). An example of this approach is shown below using a protected-propionyl hydrazide.

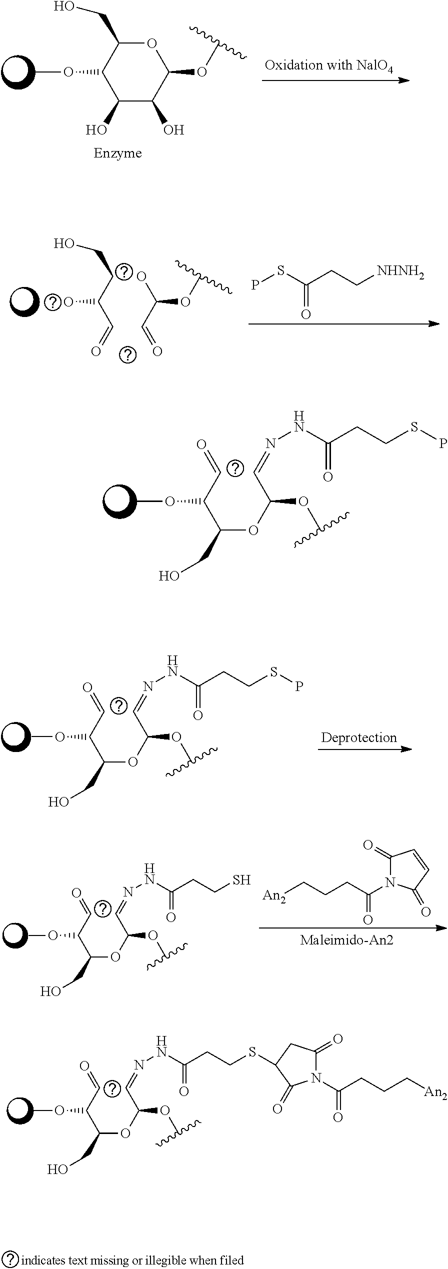

Another example of this approach is shown below.

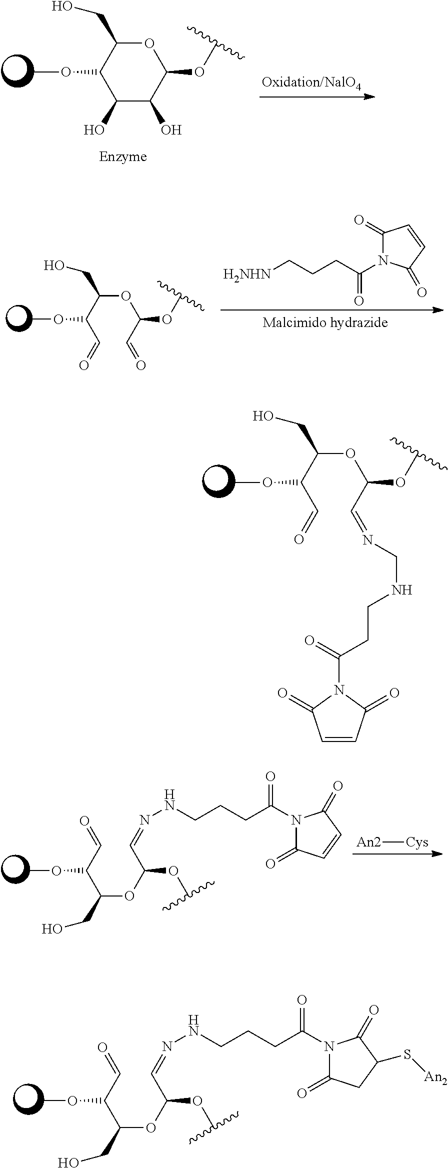

### Example 8

### Methods for Conjugation of IDS with Ant by Click Chemistry

Amino Acid sequence of iduronate-2-sulfates with possible conjugation sites highlited, i.e. lysine and N-terminal residues.

```
                10  20  30  40  50  60
MPPPRTGRGL LWLGLVLSSV CVALGSETQA NSTTDALNVL LIIVDDLRPS LGCYGDKLVR
                70  80  90  100  110  120
SPNIDQLASH SLLFQNAFAQ QAVCAPSRVS FLTGRRPDTT RLYDFNSYWR VHAGNFSTIP
              130  140  150  160  170  180
QYFKENGYVT MSVGKVFHPG ISSNHTDDSP YSWSFPPYHP SSEKYENTKT CRGPDGELHA
              190  200  210  220  230  240
NLLCPVDVLD VPEGTLPDKQ STEQAIQLLE KMKTSASPFF LAVGYHKPHI PFRYPKEFQK
              250  260  270  280  290  300
LYPLENITLA PDPEVPDGLP PVAYNPWMDI RQREDVQALN ISVPYGPIPV DFQRKIRQSY
              310  320  330  340  350  360
FASVSYLDTQ VGRLLSALDD LQLANSTIIA FTSDHGWALG EHGEWAKYSN FDVATHVPLI
              370  380  390  400  410  420
FYVPGRTASL PEAGEKLFPY LDPFDSASQL MEPGRQSMDL VELVSLFPTL AGLAGLQVPP
              430  440  450  460  470  480
RCPVPSFHVE LCREGKNLLK HFRFRDLEED PYLPGNPREL IAYSQYPRPS DIPQWNSDKP
              490  500  510  520  530  540
SLKDIKIMGY SIRTIDYRYT VWVGFNPDEF LANFSDIHAG ELYFVDSDPL QDHNMYNDSQ
              550
GGDLFQLLMP
```

### Compound Structures

### Angiopep2 Sequence

H₂N-Thr-Phe-Phe-Tyr-Gly-Gly-Ser-Arg-Gly-Lys-Arg-
Asp-Asp-Phe-Lys-Thr-Glu-Glu-Tyr-COOH

### Azido-an2 (N-Terminus)

The structure of Azidobutyryl-An2 (Azido-An2) with an N-terminal azide group is shown below. This compound was made by standard solid phase synthesis methods.

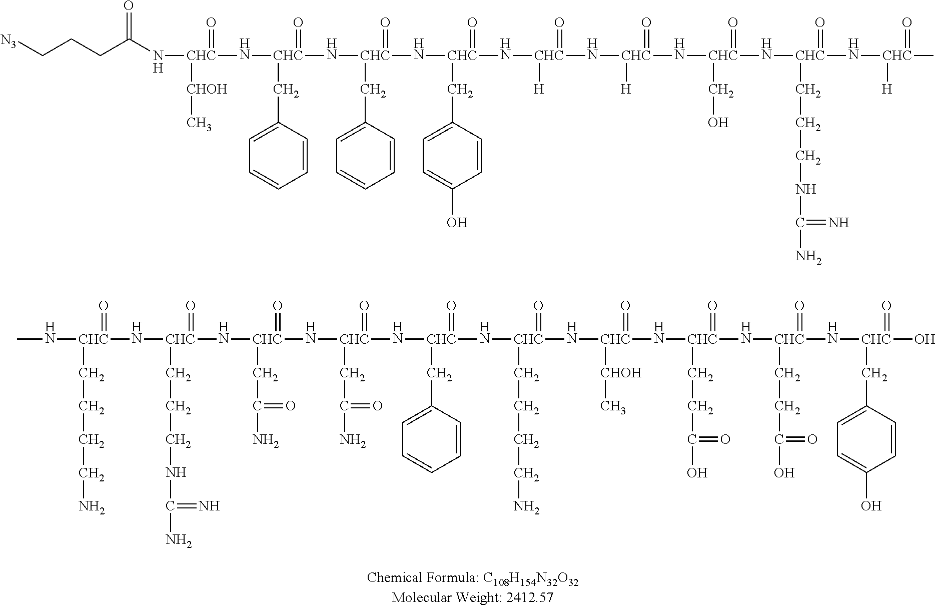

### An2-Azido (C-Terminus)

The structure of An₂-[Lys²⁰-N₃] (AN2-Azido) with a C-terminal azide group is shown below. This compound was made by standard solid phase synthesis methods.

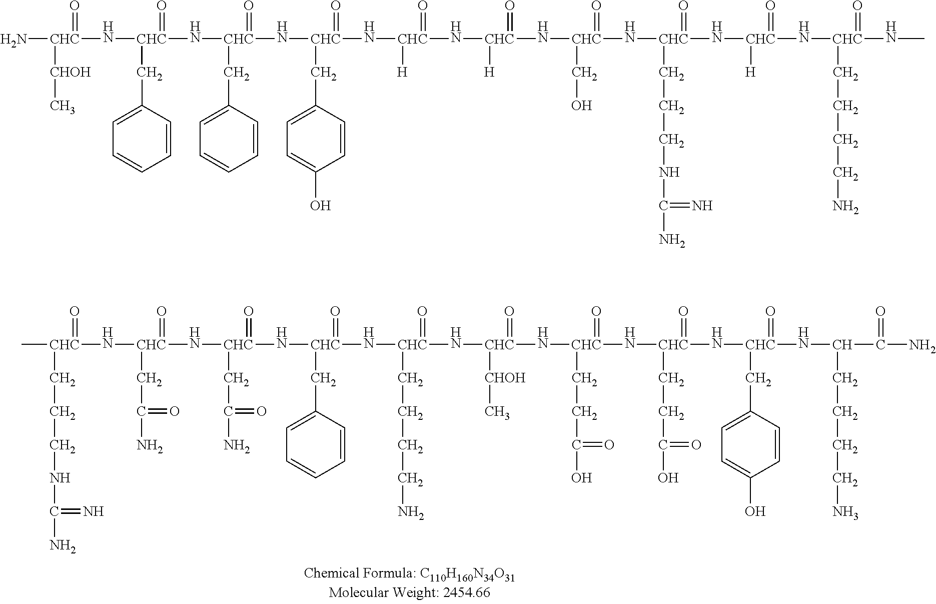

### Schematic Structure:

The structure of IDS-BCN-Butyryl-An₂ (70-56-1B and 70-56-2B) showing the conjugation on N-terminal of azidobutyryl-Angiopep-2 using BCN linker and click chemistry is shown below.

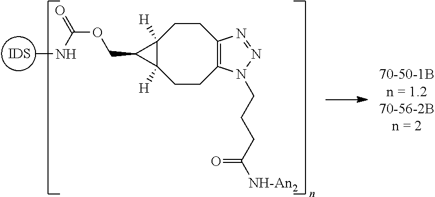

The structure of An₂-[Lys²⁰]-MFCO-IDS (70-66-1B) showing the conjugation on C-terminal of Angiopep-2-Lys²° using MFCO linker and click chemistry is shown below.

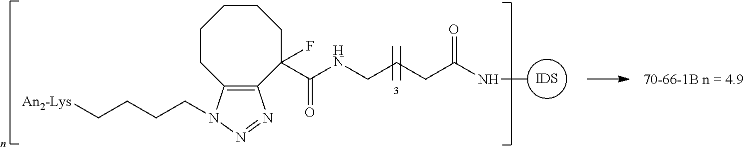

The structure of An₂-[Lys²⁰]-BCN-IDS (68-32-2) showing the conjugation on C-terminal of Angiopep-2 Lys²° using a BCN linker is shown below.

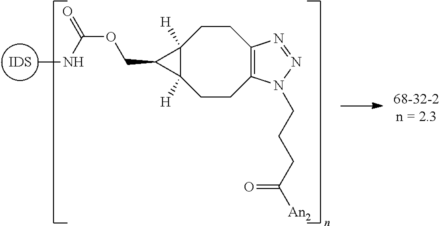

### Synthesis Scheme for 70-56-1B and 70-56-2B

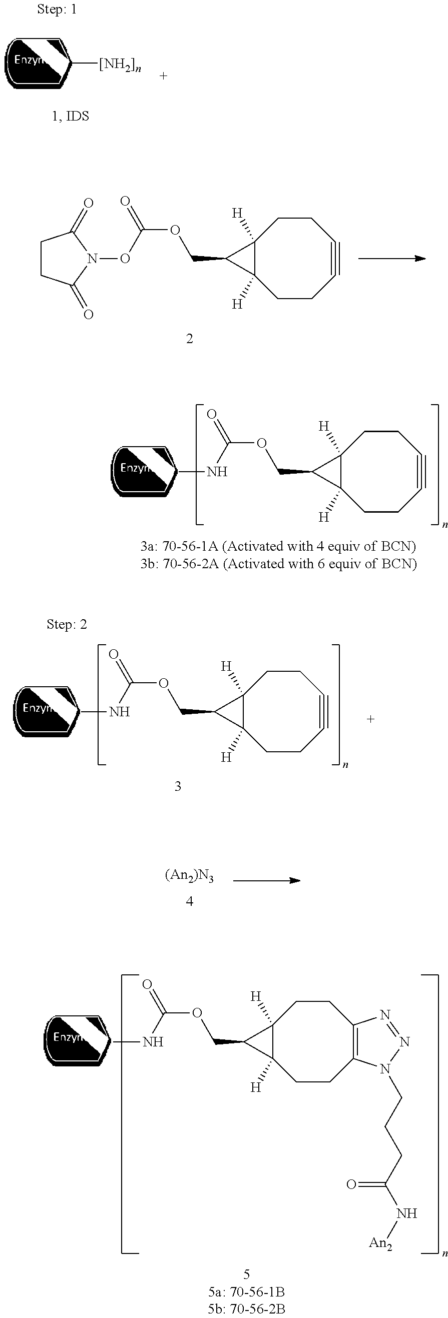

### Step: 1-Modification of IDS Lysine

BCN: bicyclo[6.1.0]nonyne

### Synthesis of 70-56-1A

To (7.24 mg, 95 nmole) of IDS (1) in phosphate buffer 20 mM at pH˜7.6, 380 nmole (4 equiv) of the BCN-N-hydroxysuccinimide ester (2) (from stock solution prepared as follows: 5.82 mg dissolved in 1000 μl of anhydrous DMSO) was added at RT for 5 h with occasional manual shaking. The modified IDS 3a, 70-56-1A was purified from the excess reagent by gel filtration with HiPrep 26/10 desalting column at 5 mL/minute with phosphate buffer 20 mM pH 7.6. The collected fractions were concentrated by Amicon ultra centrifugal filter (limit 10 kDa, 3000 rpm) to 3.8 mL (6.5 mg, yield 90%). The modified IDS 70-56-1A (3a) was recovered and was used for the next conjugation step with azidoAn2 (N-terminus) (4).

Step: 2-Conjugation of Modified IDS with Azido an2 (N Terminus)

### Synthesis of (70-56-1B)

To modified IDS derivative (3a) (6.5 mg, 85.2 nmole), 8 equiv of azidoAn2 (N-terminus) (4) was added. The solution was manually shaken, wrapped on aluminum foil and left overnight at RT. The conjugate (5) was then purified by Q Sepharose 1 mL column using 20 mM TRIS at pH7 as binding buffer whereas 20 mM TRIS and 500 mM NaCl at pH 7.0 was used as eluent buffer. The conjugate was isolated and was exchanged with IDS buffer (1×: 137 mM NaCl, 17 mM NaH₂PO₄, 3 mM Na₂HPO₄, at pH˜6) by washing 5 times 15 mL with Amicon ultra centrifugal filter (10 kDa cut-off, 3000 rpm) and was concentrated to 2.5 mL to obtain 70-56-1B (6 mg, yield 83%).

### Synthesis of 70-56-2A

To 7.24 mg (95 nmole) of IDS (1) in phosphate buffer 20 mM at pH˜7.6, 570 nmole (6 equiv) of the BCN-N-hydroxysuccinimide ester (2) was added at RT for 5 h with occasional manual shaking. The activated IDS 70-56-2B (3b) was purified from the excess reagent by gel filtration with HiPrep 26/10 desalting column at 5 mL/minute with phosphate buffer 20 mM pH 7.6. The collected fractions were concentrated by Amicon ultra centrifugal filter (10 kDa, 3000 rpm) to 3.5 mL, (6.5 mg, yield 90%). The modified IDS 3b, 70-66-2A was recovered which was used for the next conjugation step with azidoAn2 (N-terminus) (4).

### Synthesis of (70-56-2B)

To modified IDS 3b, 70-56-2A (6.5 mg, 85.2 nmole), 12 equiv of azidoAn2 (N-terminus) (4) were added. The solution was manually shaken and wrapped on aluminum foil and left overnight at RT. The conjugate (5) was purified by Q Sepharose 1 mL column using 20 mM TRIS buffer at pH 7 as binding buffer and 20 mM TRIS and 500 mM NaCl at pH 7.0 was used as eluent buffer. The conjugate was isolated and was exchanged with IDS buffer (1×: 137 mM NaCl, 17 mM NaH₂PO₄, 3 mM Na₂HPO₄, at pH˜6) by washing 5 times 15 mL with Amicon ultra centrifugal filter (10 kDa limit, 3000 rpm) and was concentrated to 3 mL to obtain 70-56-2B (6 mg, 83%).

### Synthesis Scheme for 70-66-1B

The synthesis scheme shown below shows the attachment of a MFCO linker to IDS and attachment of An₂-[Lys²⁰-N₃] (azidoAn2) to the MFCO linker via the amino group of a terminal lysine in Angiopep-2.

### Synthesis Scheme for 70-66-1B

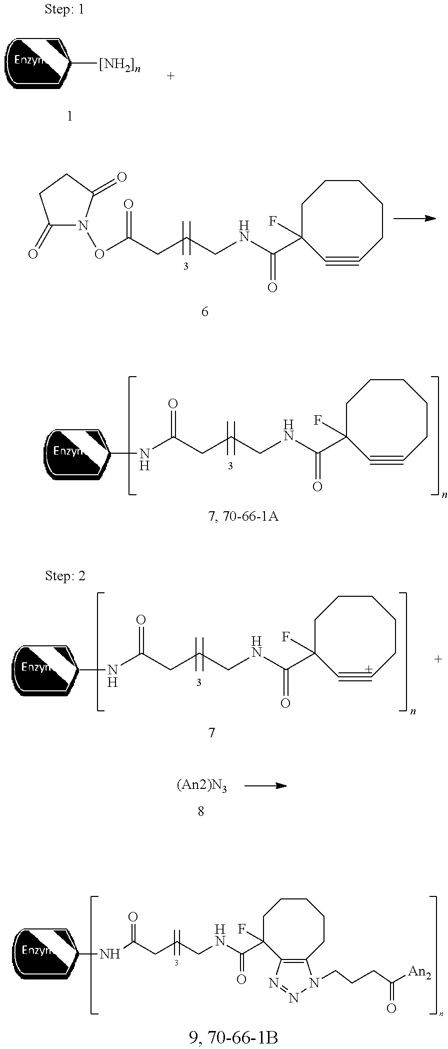

### Step: 1-Modification of IDS Lysine

### 6, MFCO: Monofluorocyclooctyne

### Synthesis of 70-66-1A

To (10.6 mg, 139 nmole) of IDS (1) in phosphate buffer 20 mM at pH˜7.6, 1112 nmole (8 equiv) of the MFCO-N-hydroxysuccinimide ester (6) (from stock solution prepared as follows: 7.6 mg dissolved in 1000 μl of anhydrous DMSO) was added and was left at RT for 5 h with occasional manual shaking. The modified IDS 70-66-1A (7) was purified from the excess reagent by gel filtration with HiPrep 26/10 desalting column at 5 mL/minute with phosphate buffer 20 mM pH 7.6. The collected fractions were concentrated by Amicon ultra centrifugal filter (10 kDa limit, 3000 rpm) to 3 mL, (9.4 mg, yield 89%). The modified IDS (7) was used for the next conjugation step with azidoAn2 (C-Tel nnus) (8).

Step: 2—Conjugation of Modified IDS with Azido an2 (C Terminus) (An₂-[Lys²⁰-N₃])

### Synthesis of (70-66-1B)

To modified IDS derivative (7), (6.1 mg, 80 nmole), 16 equiv of azidoAn2 (C-terminus) (8) were added. The solution was manually shaken and wrapped on aluminum foil and left overnight at RT. The conjugate (9) was purified by Q Sepharose 1 mL column using 20 mM TRIS at pH 7 as binding buffer whereas 20 mM TRIS and 500 mM NaCl at pH 7.0 was used as eluent buffer. The conjugate was isolated and was exchanged with IDS buffer (1×: 137 mM NaCl, 17 mM NaH₂PO₄, 3 mM Na₂HPO₄ at pH˜6) by washing 5 times 15 mL with Amicon ultra centrifugal filter (10 K mW, 3000 rpm) and was concentrated to 2.5 mL to obtain 70-66-1B (6.1 mg, 100%).

Synthesis scheme for 68-32-2

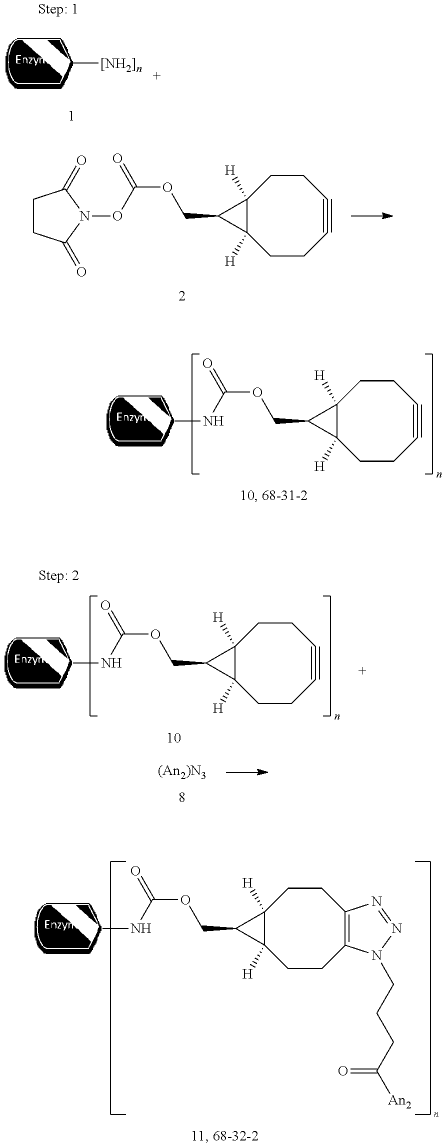

BCN: bicyclo[6.1.0]nonyne

### Step: 1-Modification of IDS Lysine

### Synthesis of 68-31-2

To (14.5 mg, 190 nmole) of IDS (1) in phosphate buffer 20 mM at pH˜7.6, 1520 nmole (8 equiv) of the BCN-N-hydroxysuccinimide ester (2) (from stock solution prepared as follows: 5.82 mg dissolved in 1000 μl of anhydrous DMSO) was added and stored at RT for 5 h with occasional manual shaking. The modified IDS (10) was purified from the excess reagent by gel filtration with HiPrep 26/10 desalting column at 5 mL/minute with phosphate buffer 20 mM pH 7. The collected fractions were concentrated by Amicon ultra centrifugal filter (limit 10 kDa, 3000 rpm) to 4 mL (14.5 mg, yield 100%). The modified IDS was recovered and was used for the next conjugation step with azido An2 (C-terminus).

Step: 2—Conjugation of Modified IDS with azido An2 (C Terminus) (An₂-[Lys²⁰-N₃])

### Synthesis of 68-32-2

To modified IDS derivative (10) (11 mg, 144.2 nmole), 16 equiv of azidoAn2 (C-terminus) were added. The solution was manually shaken and wrapped on aluminum foil and left overnight at RT. The conjugate (11) was purified by Q Sepharose lmL column using 20 mM TRIS at pH 7 as binding buffer where as 20 mM TRIS and 500 mM NaCl at pH 7.0 was used as eluent buffer. The conjugate was isolated and was exchanged with IDS buffer (1×: 137 mM NaCl, 17 mM NaH₂PO₄, 3 mM Na₂HPO₄ at pH˜6) by washing 5 times 15 mL with Amicon ultra centrifugal filter (10 K mW, 3000 rpm) and was concentrated to 2.5 mL to obtain 68-32-2 (10 mg, 91%).

Protocol for IDS Enzymatic Specific Activity (Modified from B-JR032-010-04)
1) Determine the concentration of proteins in the standard substance JR-032 and conjugates) by microBCA.
2) Preparation of the Test Solution:

Dilute JR-032 and conjugates 1/200 in Triton-X100 containing diluted buffer.
3) Prepare Standard Solution by diluting lmL 4-MU Stock Solution (0.01 mol/L) in 11.5 mL of Triton-X100 containing buffer (final concentration 800 μmol/L).
4) Prepare serial dilutions of Standard Solution by diluting 500 μL of 800 μmol/L in 500 μL of Triton X100 containing buffer to make a 400 μmol/L Standrad Solution. Repeat the process to have the following dilutions: 800, 400, 200, 100, 50, 25, 12.5 and 6.25 μmol/L.
5) Distribute 10 μL each of the blank solution (Triton-X100 containing diluted buffer) in 2 wells (n=2), standard solution (6.25 μmol to 800 mol/L) in 2 wells (n=2) and the sample solution in 4 wells each (n=4) of a microplate, respectively.
6) To each well, add 100 μL of the substrate solution (4-MUS) and mix gently.
7) Cover the plate and place in an incubator adjusted to 37° C.
8) Add 190 μL of the stop solution to each well exactly after 60 minutes and mix to stop the reaction.
9) Set the plate in the fluorescence plate reader and determine fluorescence intensity at excitation wavelength of 355 nm and detection wavelength of 460 nm.
10) Perform the same measurement with the reference material if comparison is required among tests.

### Method of Calculation:

11) Concentration of 4-MU produced from the sample solution

Determine the concentration of 4-MU, Cu (μmol/L), produced from the sample solution using the following formula.

$$Cs = \frac{w}{176.17} \times \frac{{10}^{6}}{50 \times 100}$$

w: Amount (mg) of 4-MU (176.17: Molecular weight of 4-MU)

Cs: Concentration (μmol/L) in the standard solution

$$Cu = Cs ( \frac{Au}{As} )$$

Au: Fluorescence intensity of the sample solution

As: Fluorescence intensity of the standard solution
12) Specific activity of the sample solution: Determine the specific activity, B (mU/mg), of the sample solution using the following formula.

$$B = \frac{\frac{Cu}{60} \times C \times \frac{50}{0.1}}{P}$$

C: Dilution factor of the desalted test substance

B: Specific activity (mU/mg)

P: Concentration (mg/mL) of proteins in the desalted test substance

### Protocol for Glycosaminoglycan (GAG) Accumulation Assay

### Materials:

Type II MPS Hunter fibroblasts (Coriell institute, GM00298)
Healthy human fibroblasts (Coriell institute, GM05659)
DMEM, fetal bovine serum (FBS)
low sulfate Ham's F-12 medium (Invitrogen, catalog #11765-054)
FBS dialysed against 0.15 M NaCl, 10000 Da MWCO (Sigma, catalog # F0392)
³⁵S-sodium sulfate (Perkin-Elmer, catalog # NEX041H002MC)

### Method:

1. MPS II (GM00298) or healthy human fibroblasts (GM05659) in 6-well dishes at 250,000 cells/well in DMEM with 10% fetal bovine serum (FBS).
   Grow for 4 days.
2.—Discard medium, wash cells with warm and sterile PBS.
   Add 1 mL/well of low sulfate F-12 medium with 10% dialysed FBS and 10 μCi ³⁵S-sodium sulfate.
   Add recombinant IDS proteins. Incubate at 37° C., 5% CO₂ for 48 h
3.—Discard medium, wash cells with cold PBS (1 mL, 5 washes).
   Lyse cells in 0.4 mL/well of 1 N NaOH.
   Heat at 60° C. for 60 min to solubilize proteins.
   Remove and aliquot for BCA protein assay.
4. Count radioactivity with a liquid scintillation counter.
5. μBCA protein assay.
6. The data are expressed as ³⁵S CPM per μg protein.

### Protocol for In Situ Brain Perfusion.

The in situ mice brain perfusion method was established in the laboratory from the protocol described by Dagenais et al., 2000. Briefly, the surgery was performed on sedated mice, injected intraperitoneal (i.p.) with Ketamine/Xylazine (140/8 mg/kg). The right common carotid artery was exposed and ligated at the level of the bifurcation. The common carotid was then catheterized rostrally with polyethylene tubing (0.30 mm i.d.×0.70 mm o.d.) filled with saline/heparin (25 U/ml) solution mounted on a 26-gauge needle. The studied molecule was radiolabeled with ¹²⁵I in the days preceding the experiment using iodo-Beads from Pierce. Free iodine was removed on gel filtration column followed by extensive dialysis (cut-off 10 kDa). Radiolabeled proteins were dosed using the Bradford assay and JR-032 as the standard.

Prior to surgery, perfusion buffer consisting of KREBS-bicarbonate buffer—9 mM glucose was prepared and incubated at 37° C., pH at 7.4 stabilized with 95% O₂: 5% CO₂. A syringe containing radiolabeled compound added to the perfusion buffer was placed on an infusion pump (Harvard pump PHD2000; Harvard apparatus) and connected to the catheter. Immediately before the perfusion, the heart was severed and the brain was perfused for 2 min at a flow rate of 2.5 ml/min. All perfusions for IDS and An2-IDS conjugates were performed at a concentration of 5 nM. After perfusion, the brain was briefly perfused with tracer-free solution to wash out the blood vessels for 30 s. At the end of the perfusion, the mice were immediately sacrificed by decapitation and the right hemisphere was isolated on ice and homogenized in Ringer/Hepes buffer before being subjected to capillary depletion.

### Capillary Depletion

The capillary depletion method allows the measure of the accumulation of the perfused molecule into the brain parenchyma by eliminating the binding of tracer to capillaries. The capillary depletion protocol was adapted from the method described by Triguero et al., 1990. A solution of Dextran (35%) was added to the brain homogenate to give a final concentration of 17.5%. After thorough mixing by hand the mixture was centrifuged (10 minutes at 10000 rpm). The resulting pellet contains mainly the capillaries and the supernatant corresponds to the brain parenchyma.

### Determination of Tracer Signal

Aliquots of homogenates, supernatants, pellets and perfusates were taken to measure their contents in radiolabeled molecules. [¹²⁵I]-samples were counted in a Wizard 1470 Automatic Gamma Counter (Perkin-Elmer Inc, Woodbridge, ON). All aliquots were precipitated with TCA in order to get the radiolabeled precipitated protein fractions. Results are expressed in term of volume distribution (m1/100 g/2 min) for the different brain compartments.

### Example 9

### Screening and Characterization of Compounds

### Screening

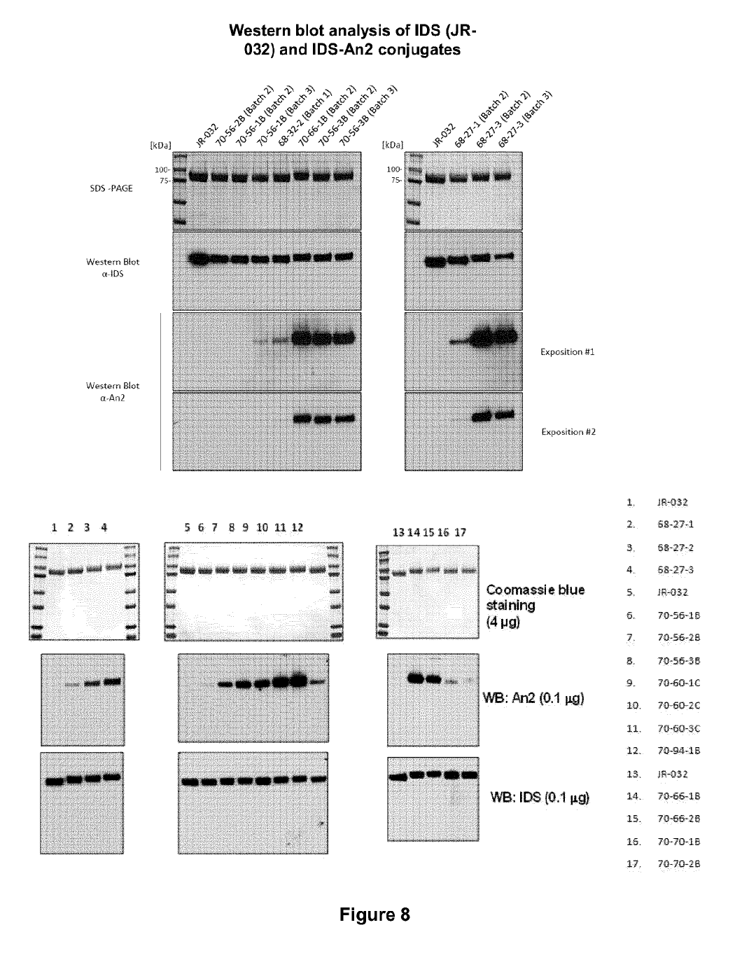

Recombinant iduronate-2-sulfatase (IDS) (JCR-032) was conjugated to An2 via lysine attachment. The IDS amino acid sequence with potential attachment sites marked is presented above in Example 8. These conjugates represent varying ratios of An2:linker to IDS. Linkers tested in this conjugation strategy were click chemistry linkers including MFCO (monofluorocyclooctyne), BCN (bicyclononyne), SATA (S-acetylthioacetate), DBCO (dibenzylcyclooctyne), and maleimido. In all cases, the ratio of An2:linker material added to the reaction is 2:1, with An2 in excess of IDS by either 4-, 6-, or 8-fold. An2 was removed from the reaction product by Q-sepharose column chromatography, and MALDI-TOF analysis was used to determine the average number of An2 incorporated on each IDS. SP-HPLC analysis was used to determine whether unconjugated IDS was present in the product. SEC analysis was used to examine the quality of the protein following conjugation. Using this method, the first series of nine conjugates were found to have evidence of aggregate formation, and the conjugation reactions were optimized and repeated to eliminate this issue. In addition, five novel conjugates were produced using other linkers. The lysine conjugates that were selected for testing for enzyme activity, GAG reduction, and in situ brain perfusion are presented in Table 3 below. Note that the number of An2 incorporated is an average as multiple species may exist in conjugation reaction products. The mass of JR-032 by MALDI TOF is 76,320 Da (11 determinations). Western blots for these conjugates are presented in FIG. 8.

TABLE 3
An2-IDS lysine conjugates selected for further analysis.

| | | | | | Mass of | | | |
|---|---|---|---|---|---|---|---|---|
| | | | | | Conjugate | Number of | | |
| IDS-An2 | | | Ratio | MW of | By Maldi | An2 | Yield | Code |
| Conjugate | Linker | An2 | (Activation:An2) | linker + An2 | Tof | Incorporated | (%) | (Name) |
| 68-27-1 | MFCO | An2 | 4:8 | 2678 | 83,362 | ~2.3¹ | 80 | ANG3404 |
| | | | | | | (2.6; 2.0) | | (IDS- |
| 68-27-2 | MFCO | An2 | 6:12 | 2678 | 88,133 | 4.4 | 65 | MFCO- |
| 68-27-3 | MFCO | An2 | 8:16 | 2678 | 90,484 | ~5.0² | 65 | Butyryl- |
| | | | | | | (5.3; 4.2; 5.5) | | An₂) |
| 70-56-1B | BCN | An2 | 4:8 | 2589 | 79,265 | ~1.2² | 83 | ANG3402 |
| | | | | | | (1.2; 1.0; 1.2) | | (IDS-BCN- |
| 70-56-2B | BCN | An2 | 6:12 | 2589 | 81,321 | ~2.4¹ | 81 | Butyryl- |
| | | | | | | (2.0; 2.8) | | An₂) |
| 70-56-3B | BCN | An2 | 8:16 | 2589 | 82,826 | ~3.0² | 80 | |
| | | | | | | (2.5; 3.2; 3.3) | | |
| 70-60-1C | SATA | An2 | 4:8 | 2570 | 80,303 | 1.5 | 84 | ANG3406 |
| 70-60-2C | SATA | An2 | 6:12 | 2570 | 82,961 | 2.6 | 80 | (IDS- |
| 70-60-3C | SATA | An2 | 8:16 | 2570 | 85,289 | 3.5 | 81 | SATA- |
| | | | | | | | | An₂) |
| 70-066-1B | MFCO | An2N3 | 8:16 | 2719 | 89,566 | ~4.9¹ | 100 | ANG3403 |
| | | (C) | | | | (4.9; 4.8) | | (An₂- |
| | | | | | | | | [Lys²⁰]- |
| | | | | | | | | MFCO- |
| | | | | | | | | IDS) |
| 70-066-2B | MFCO | An2N3 | 8:16 | 2678 | 89,374 | 4.9 | 93 | ANG3404 |
| | | (N) | | | | | | (IDS- |
| | | | | | | | | MFCO- |
| | | | | | | | | Butyryl- |
| | | | | | | | | An₂) |
| 70-070-1B | Maleimide | An2Cys | 8:16 | 2675 | 78,562 | 0.8 | 100 | ANG3407 |
| | | (C) | | | | | | (An₂- |
| | | | | | | | | [Cys²⁰]- |
| | | | | | | | | maleimido- |
| | | | | | | | | IDS) |
| 70-070-2B | Maleimide | An2Cys | 8:16 | 2675 | 78,773 | 0.9 | 100 | ANG3408 |
| | | (N) | | | | | | (IDS- |
| | | | | | | | | maleimido- |
| | | | | | | | | Cys-An₂) |
| 70-094-1B | DBCO | An2N3 | 8:16 | 2728 | 79,840 | 1.3 | 100 | ANG3405 |
| | | (N) | | | | | | (IDS- |
| | | | | | | | | DBCO- |
| | | | | | | | | Butyryl- |
| | | | | | | | | An₂) |
| 68-32-2 | BCN | An2N3 | 8:16 | 2589 | 83,738 | 2.3 | TBD | ANG3401 |
| | | (C) | | | | | | (An₂- |
| | | | | | | | | [Lys²⁰]- |
| | | | | | | | | BCN-IDS) |

¹= average of two values.
²= average of three values.

These conjugates were evaluated to determine:
1. An2 incorporation (range of 1-5 An2/IDS)
2. no evidence of aggregation by SEC
3. no more than two major peaks by SP-analysis

A cysteine strategy was also employed in an effort to limit (and standardize) the number of An2 incorporated to one per IDS, however, no more that 50% of IDS conjugation with An2 was attained using a range of conditions including up to 20 equivalents of An2. Moreover, the conjugation reaction products showed a 50% loss of enzymatic activity, suggesting that the conjugated material was inactive. Thus, the lysine approach was favored.

### Profiling

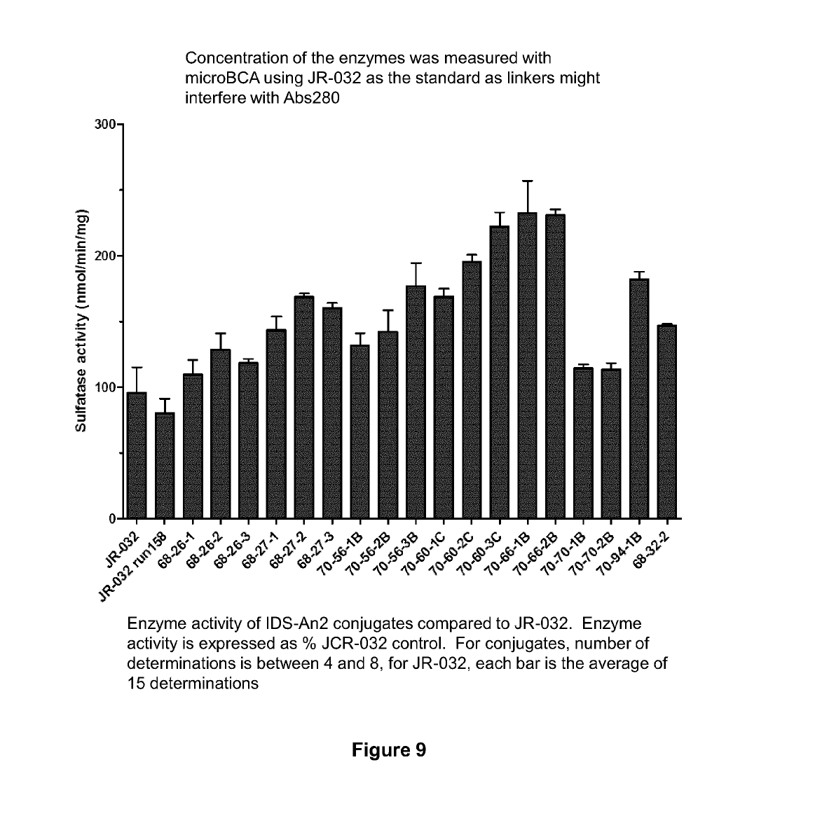

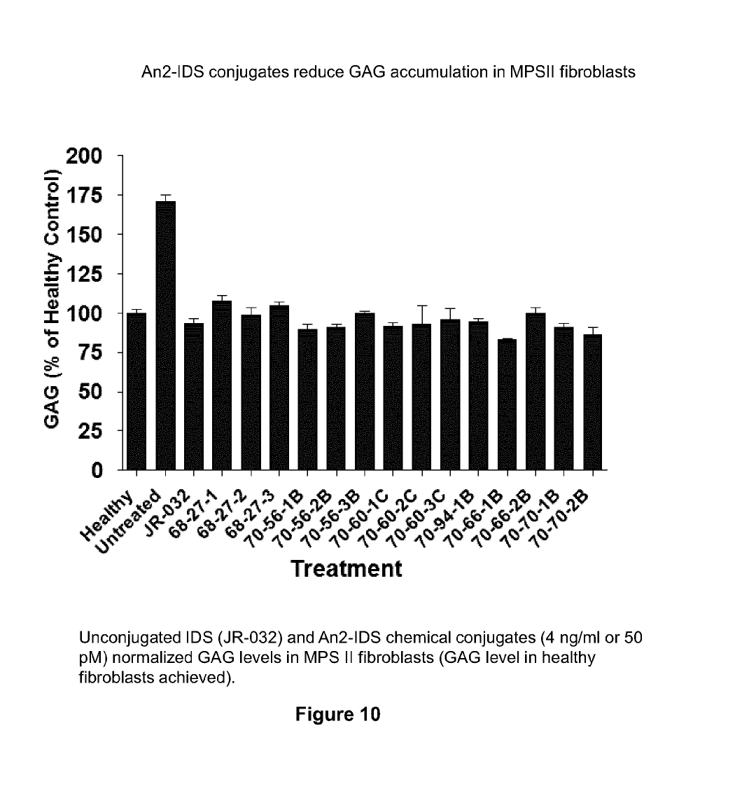

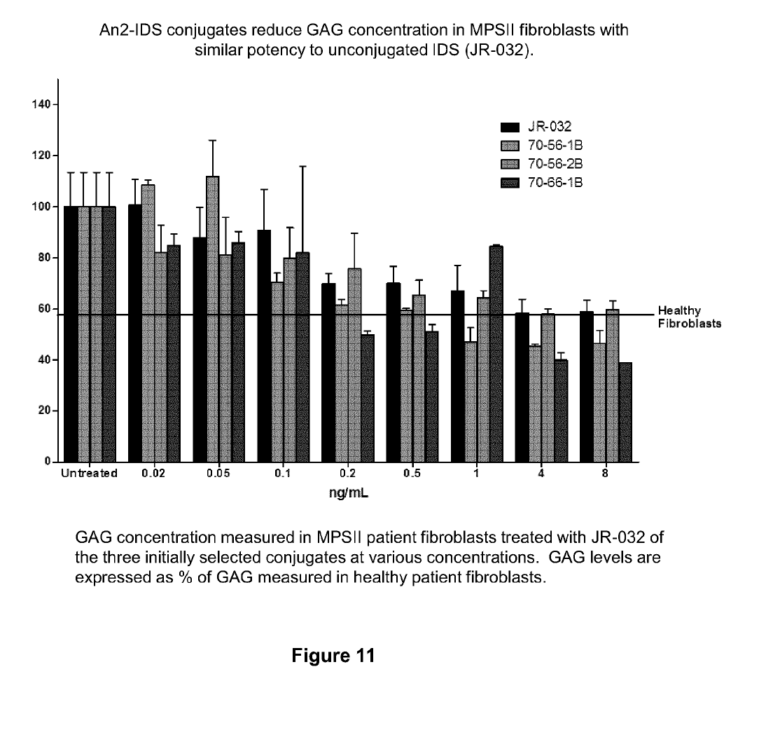

The lysine conjugates were subjected to in vitro enzyme assays with JR-032 as a control. Experimental details are described above. All conjugates retain enzyme activity (see FIG. 9). In some cases, measured activity exceeds that of native IDS. This may result from interference in the protein quantification assay, leading to a lower calculated protein concentration and higher activity/protein. To confirm enzymatic activity with a functional endpoint, the conjugates were assayed for efficacy at reducing GAG levels in fibroblasts from MPSII patients. At a concentration of 4 ng/ml (50 pM), GAG levels are reduced to levels observed in non-disease fibroblasts, similar to that observed with JR-032 (see FIGS. 10 and 11).

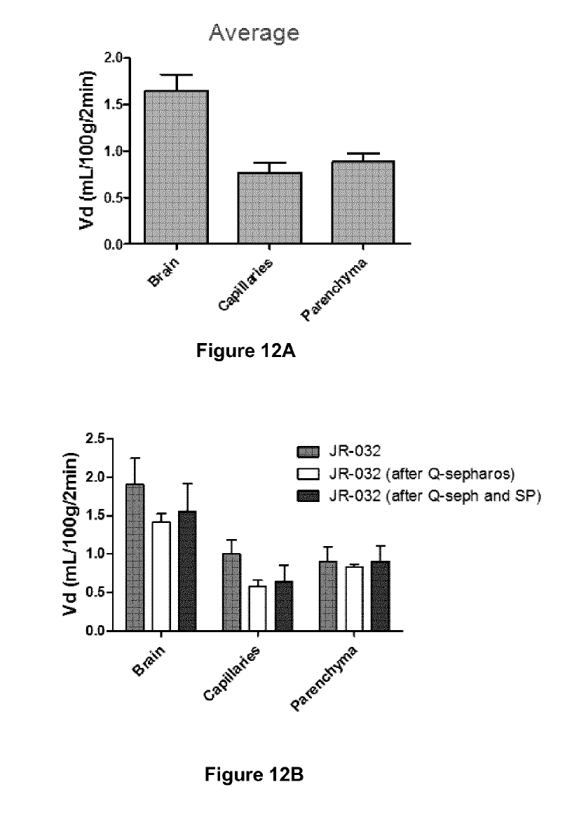

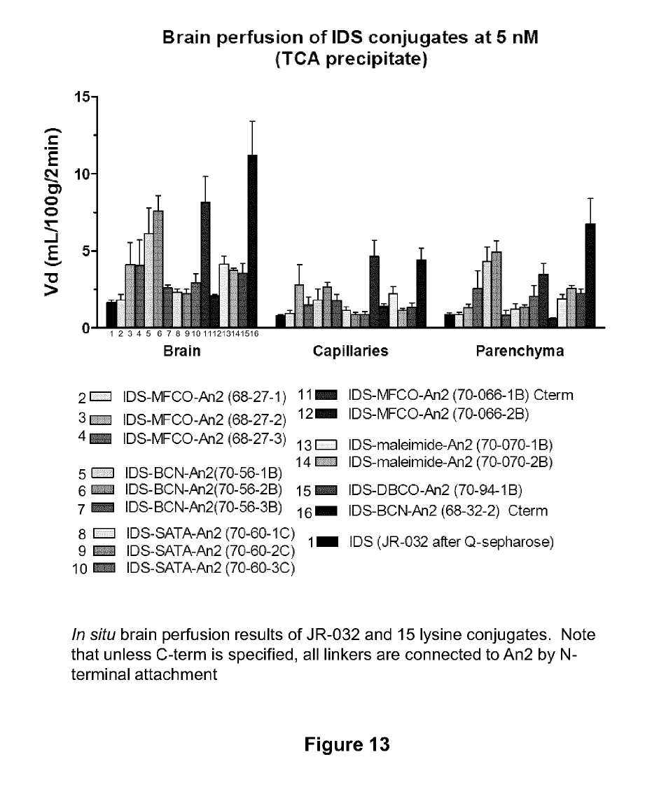

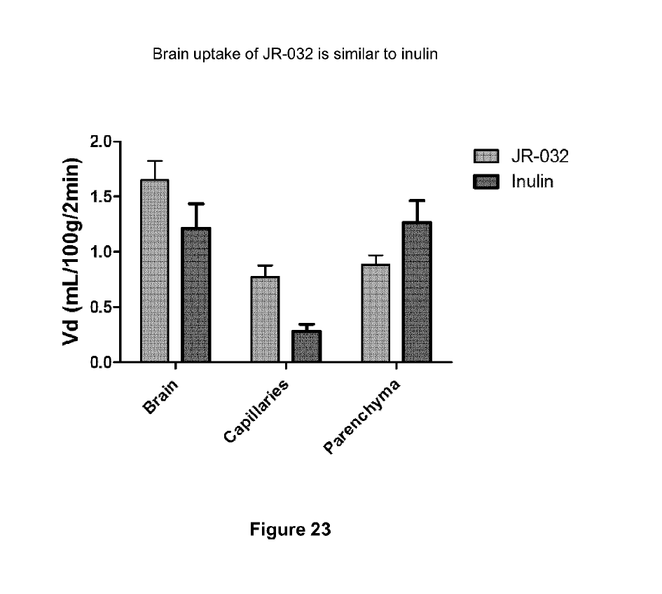

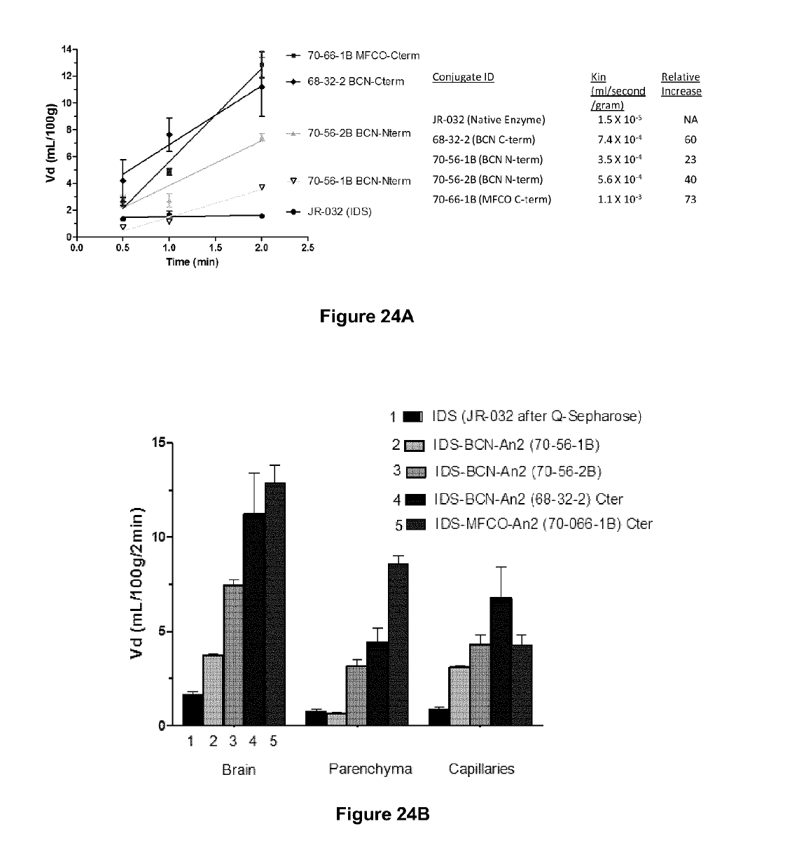

To determine whether conjugation confers an advantage with respect to brain penetration, conjugates were radio-iodinated and tested in the in situ brain perfusion assay in mouse. In this experiment, enzyme (5 nM) is delivered via the carotid artery, thereby maximizing the amount delivered selectively to brain. Following a two minute exposure, the brain was perfused with saline to remove circulating enzyme. Upon removal of the brain, a capillary depletion protocol was used to separate capillary-associated and parenchymal fractions. Radioactivity was counted to quantify the volume of distribution of the test article. JR-032 was used as a control in all experiments and its results were pooled to generate a single control value. As no decision-driving differences between the conjugates were observed with respect to enzyme activity and GAG reduction, the result of this in vivo BBB-penetration assessment was the main driver for compound selection. FIGS. 12 and 13 show the brain distribution of JR-032 and 15 conjugates respectively at a single time point (2 minutes). A comparison of the brain distribution of JR-032 relative to inulin is provided in FIG. 23.

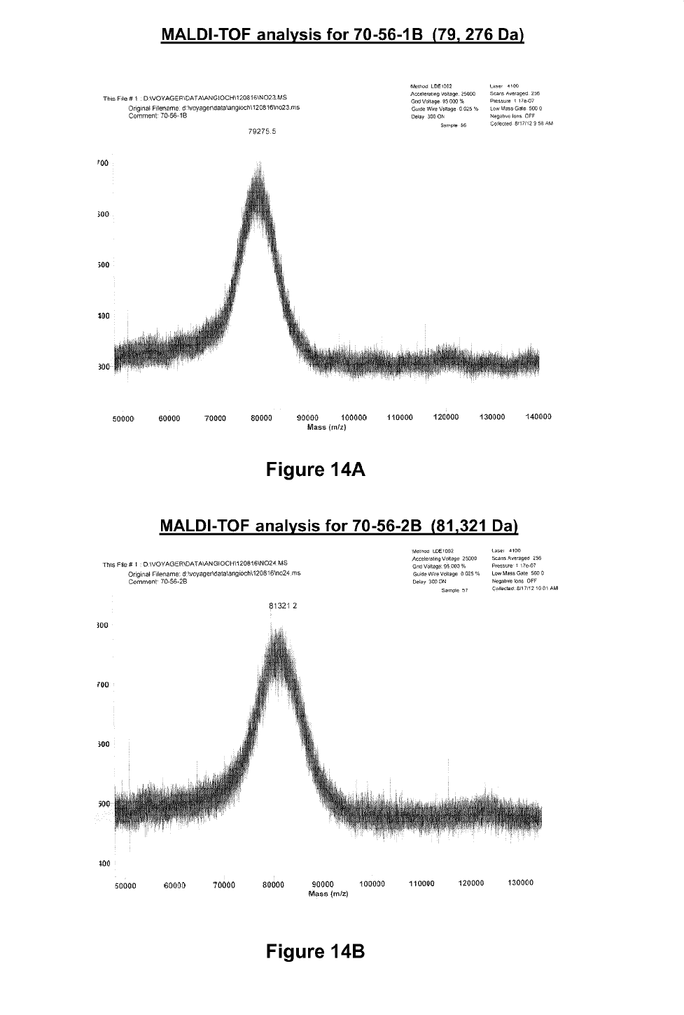

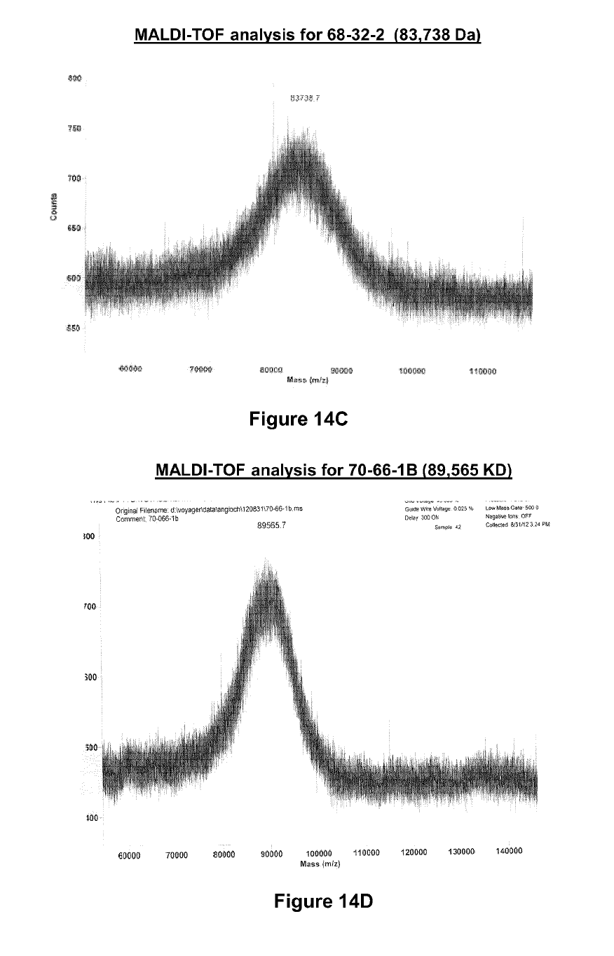

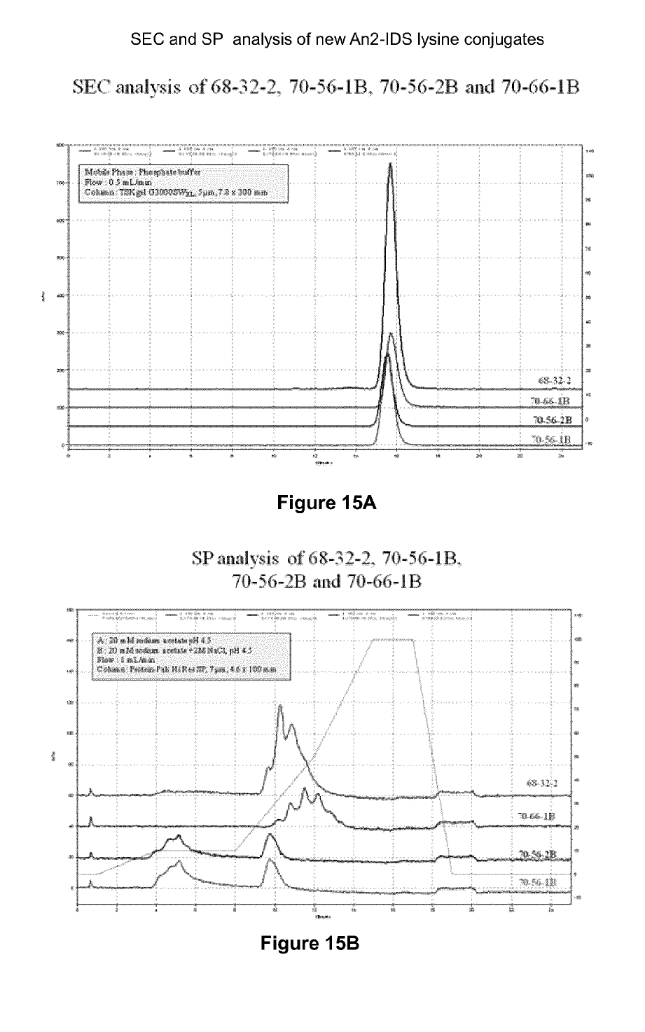

FIGS. 14A, 14B, 14C, and 14D show MALDI-TOF analyses of 70-56-1B, 70-56-2B, 68-32-2, and 70-66-1B respectively. FIGS. 15A and 15 B show SEC and SP analyses of 68-32-2, 70-56-1B, 70-56-2B, and 70-66-1B. The structures of these conjugates and a summary of the synthetic protocols are provided above. The average numbers of An2 incorporated into 68-32-2, 70-66-1B, 70-56-2B, and 70-56-1B are 2.3, 4.9, 2.4, and 1.2, respectively. No unconjugated JR-032 is detected in these analyses. Two peaks, representing two populations of An2-IDS, are visible for each conjugate, one eluting at 4-5 minutes and the second at 10 minutes. Purification of similarly spaced peaks for a different An2-IDS conjugate has been demonstrated.

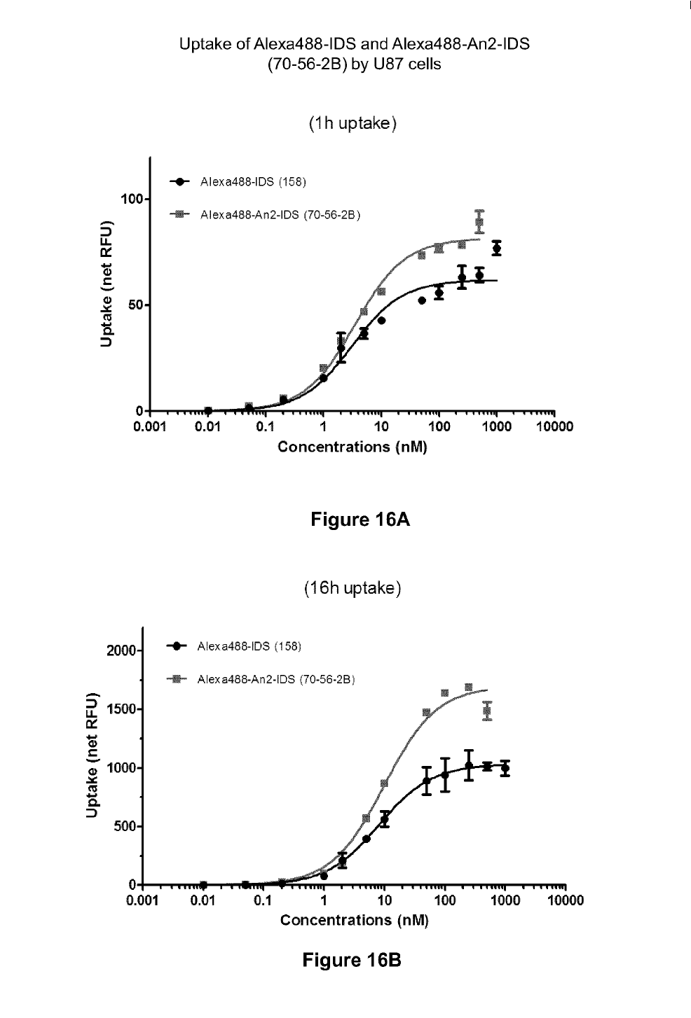

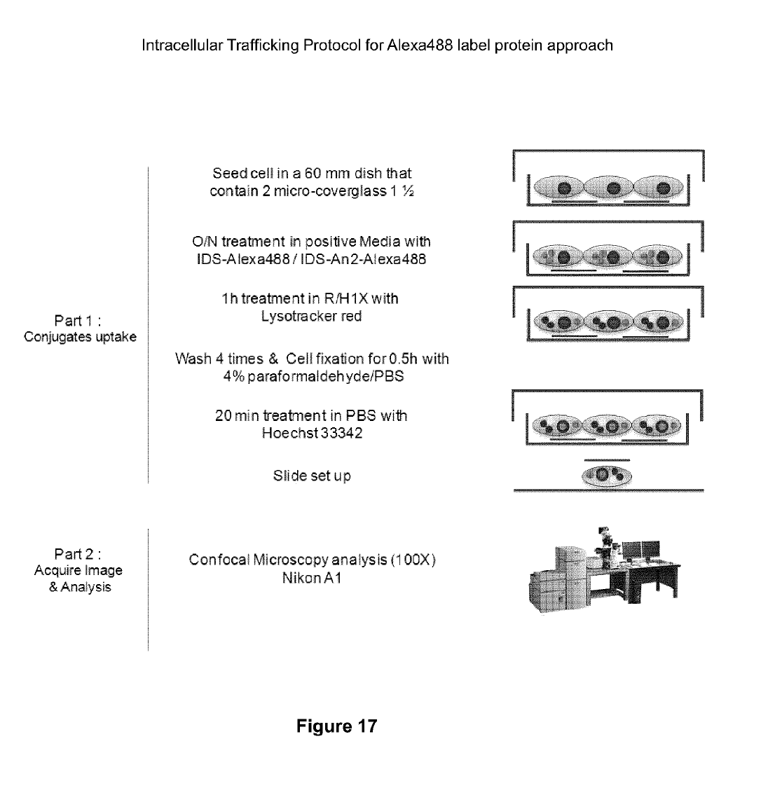

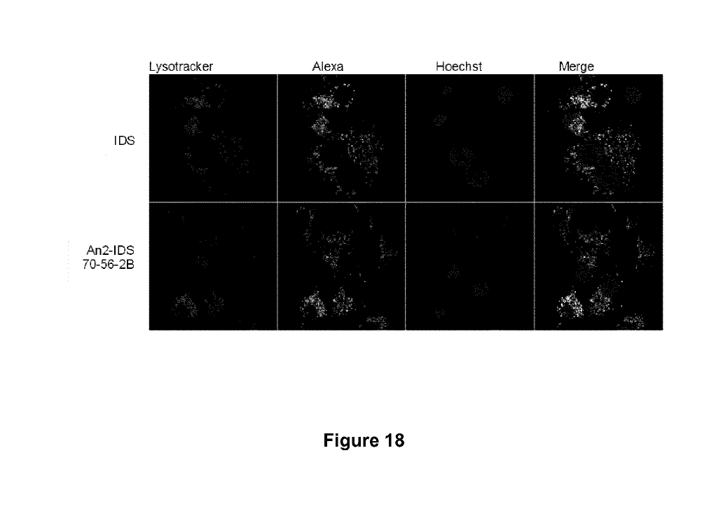

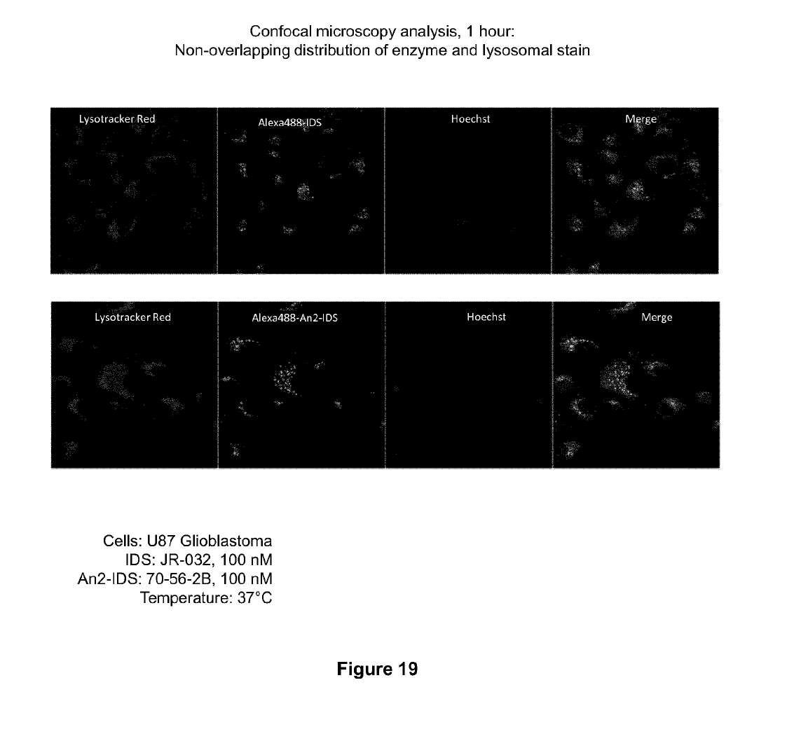

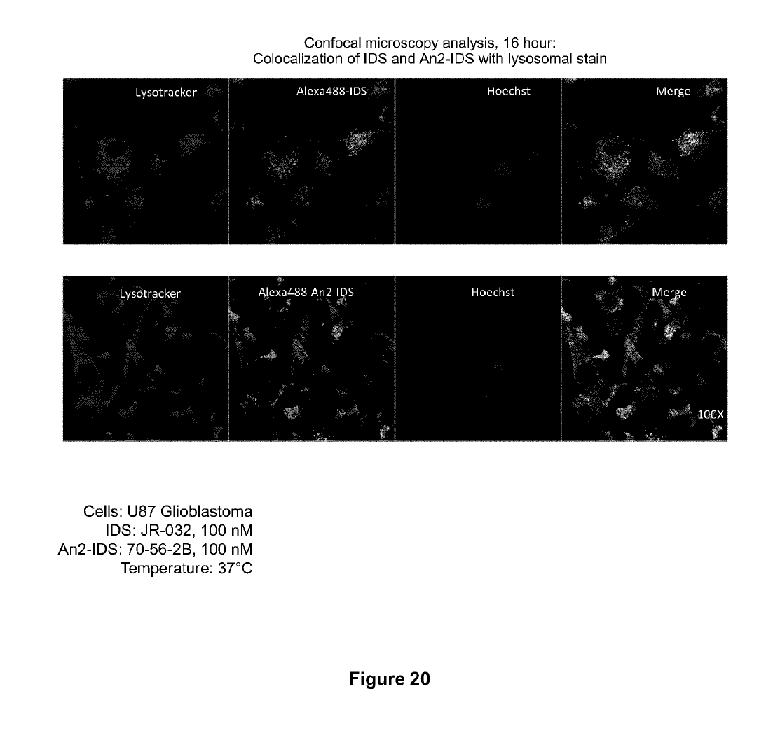

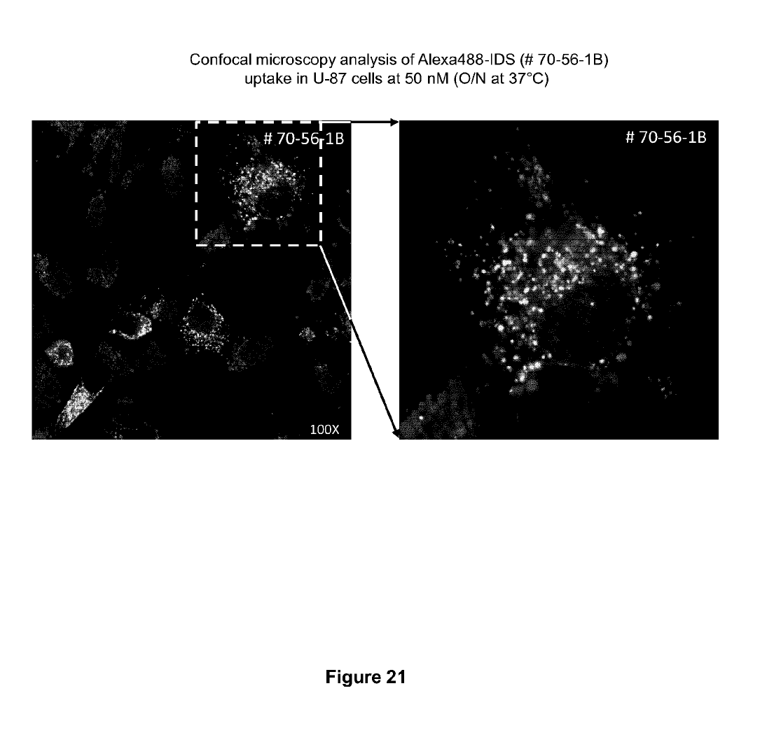

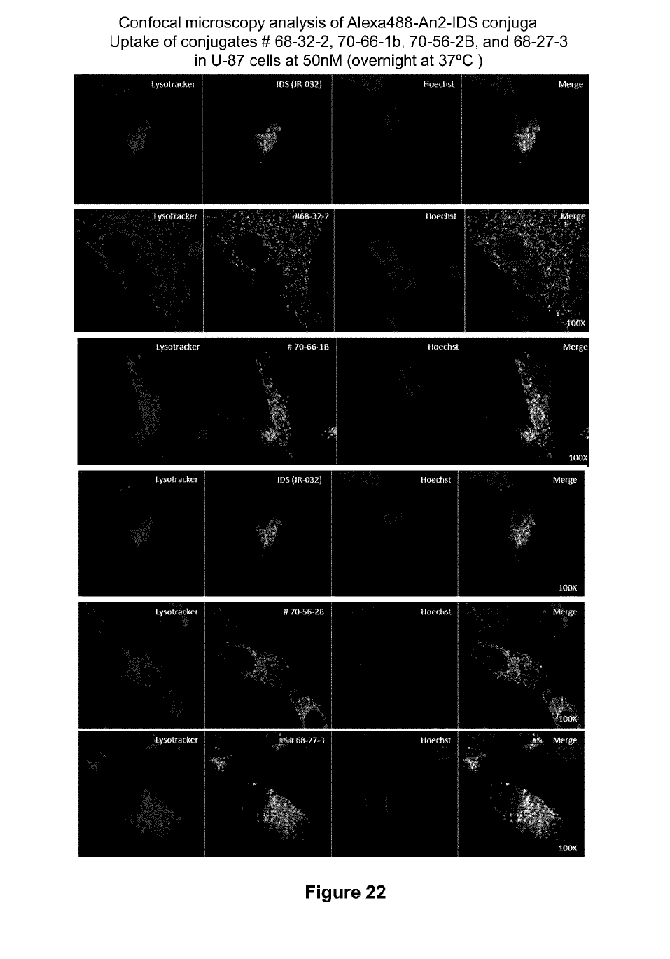

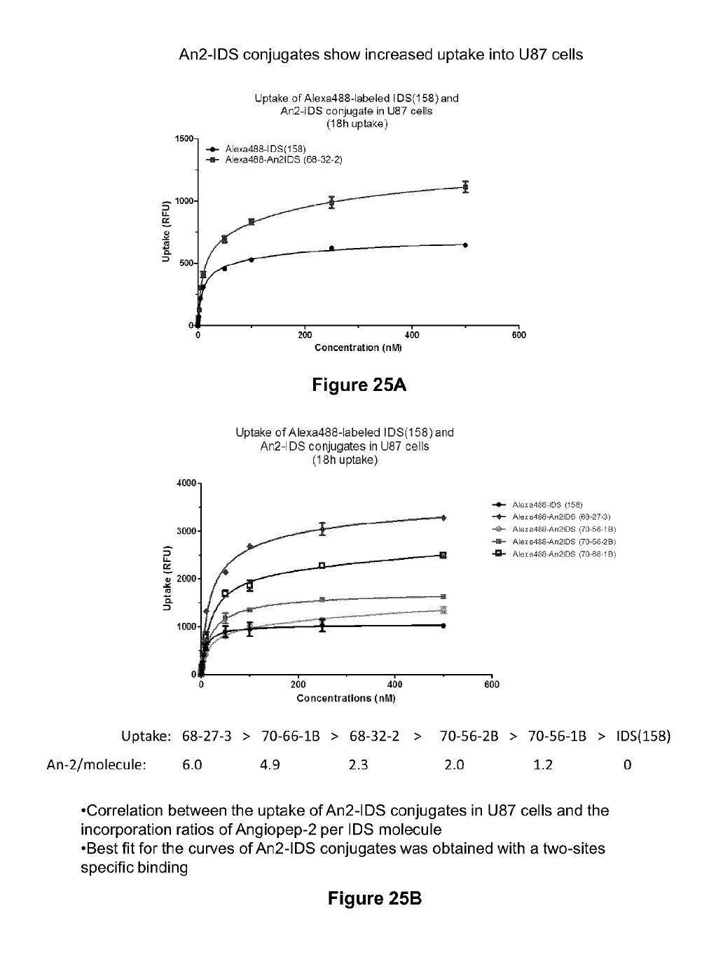

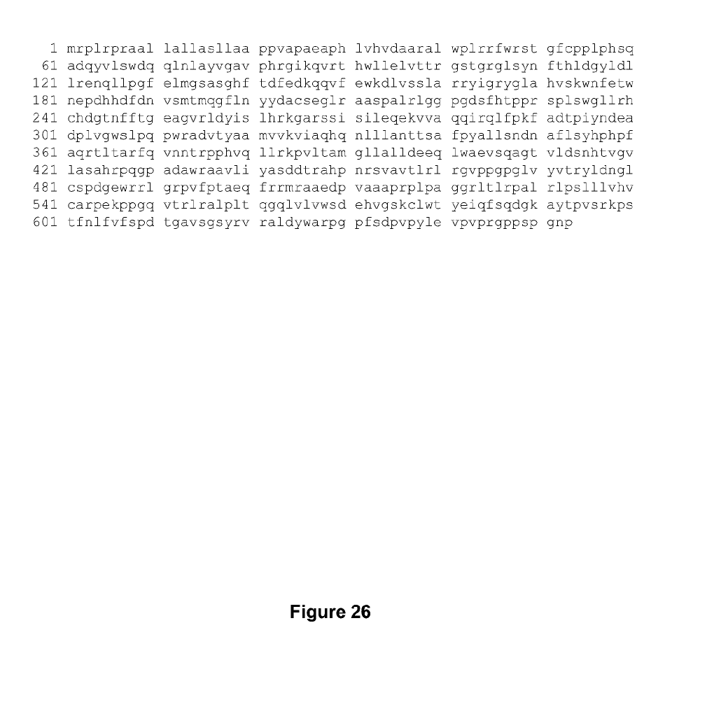

The conjugation products were labeled with Alexa 488 dye and used in trafficking studies in U87 cells to compare their localization with that of the lysotracker dye. A schematic of the microscopy experiment is provided in FIG. 17 and results of the confocal microscopy of 68-32-2, 70-56-1B, 70-56-2B, and 70-66-1B conjugates, labeled with Alexa 488 dye, showing their localization relative to the lysotracker dye are shown in FIGS. 18-22. Colocalization of a conjugate with the lysotracker dye indicated the presence of that conjugate in acidic lysosomes. FIG. 16 shows quantitation of data showing that the entry of both conjugated and native JR-032 was observed following a 1 hour or 16 hour (FIG. 16) incubation. The uptake EC₅₀ is approximately 10 nM for both enzymes, with a higher maximal uptake demonstrated for 70-56-2B. The protocol for this experiment is provided above. Further data supporting the uptake of An2-IDS into U-87 cells and the brain is shown in FIGS. 24 and 25.

### Example 10

### Synthesis of IDS-Angiopep-2 Conjugates with Cleavable Linkers

An2 is conjugated to IDS via a disulfide containing cleavable linker via the two schemes shown below. In the first scheme the lysine side chain of IDS is reacted with a SPDP linker to generate modified IDS. The modified IDS is reacted with An₂-Cys-SH to attach the An2 via the S moiety of the C-terminal cysteine of An₂-Cys to generate an IDS-An₂ conjugate.

In the second scheme, IDS is reacted with a SATA linker followed by reaction with hydroxylamine to generate modified IDS. The N-terminal lysine of An₂ is reacted with SPDP to generate a modified An₂. The modified IDS is reacted with the modified An₂ to attach the An₂, via the N-terminal amino group of An₂, to IDS to generate a IDS-An₂ conjugate.

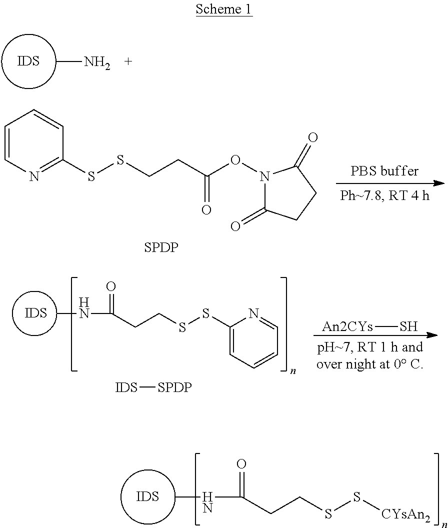

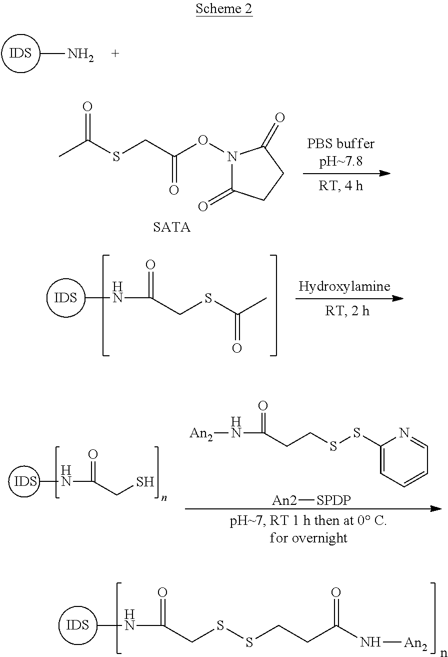

### Example 11

### IDUA Fusion Protein Constructs and Expression in Mammalian Cells

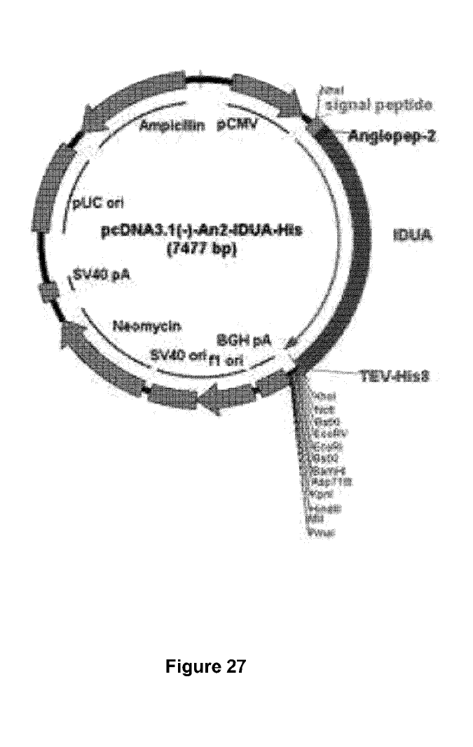

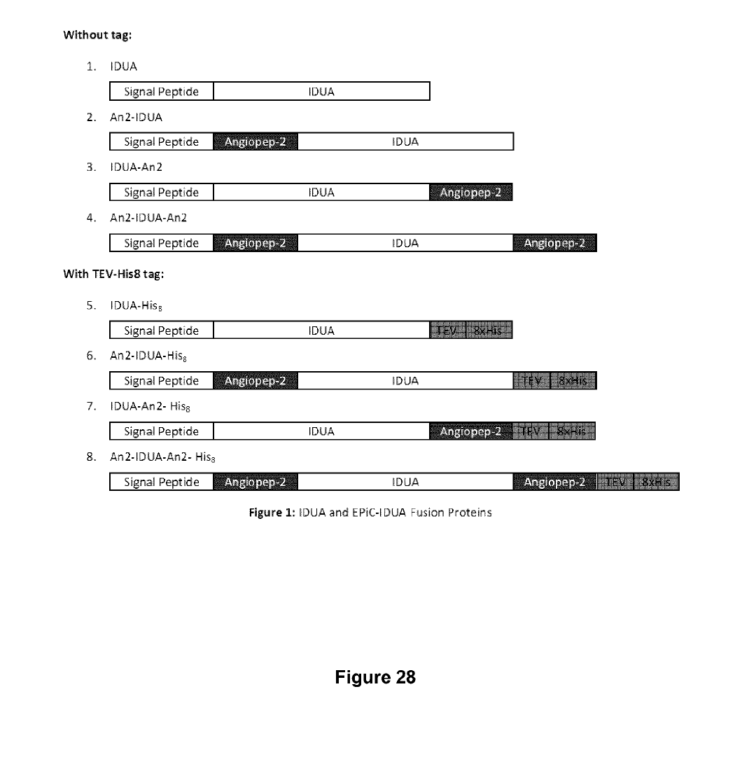

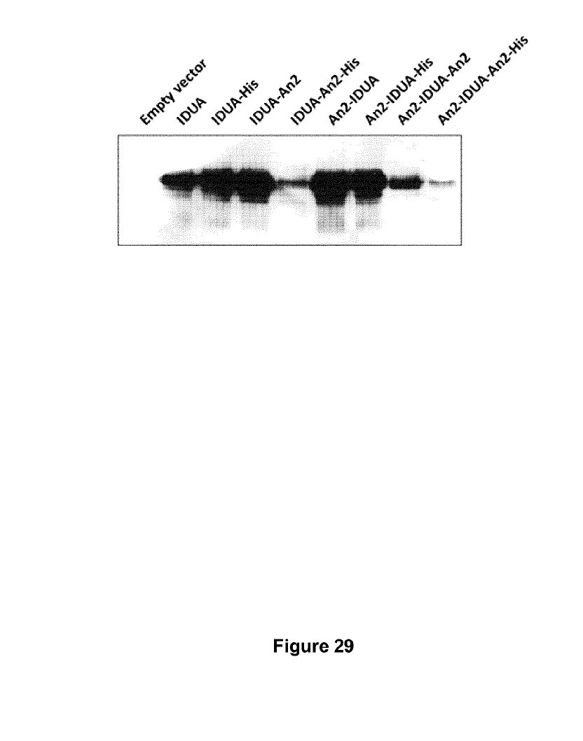

The full-length human IDUA cDNA clone (NM_{—}000203.2) was obtained from OriGene. The coding sequence for Angiopep-2 (An2) and the coding sequence for a TEV cleavable histidine-tag were produced by PCR. cDNA constructs with and without a His-tag were subcloned in suitable expression vectors such as pcDNA3.1 (Qiagen GigaPrep) (FIG. 27) under the control of the CMV promoter. IDUA and EPiC-IDUA plasmids of all studied candidates (with/without a cleavable Histidine tag) were transfected into commercially available CHO—S expression systems (FreeStyle™ Max expression systems, Invitrogen) using polyethylenimine (PEI) as transfection reagent and Freestyle CHO expression medium (serum-free medium, Invitrogen). In these systems the cells are grown in suspension and, following transfection of the expression plasmid, the fusion proteins are secreted in the culture media. Culture and transfection parameters were optimized for maximal expression in small-scale experiments (30 ml). The expression of recombinant fusion proteins in the cell culture media was monitored by measuring IDUA enzyme activity using the fluorogenic substrate 4-methylumbelliferyl α-L-iduronide and western blotting using anti-IDUA, anti-Angiopep-2, or anti-hexahistidine antibodies. Eight IDUA and EPiC-IDUA fusion proteins were designed, as shown in FIG. 28, and expressed in CHO—S cells as shown by the expression levels detected in the cell media by western blot (FIG. 29). Good expression levels were observed except for the following constructs: IDUA-An2-His, An2-IDUA-An2, and An2-IDUA-An2.

### Example 12

### Expression and Purification of IDUA Fusion Constructs

The following steps describe the optimized conditions for transfection, expression, and purification of IDUA fusion proteins.

Transfection was performed as follows. The day before transfection, split CHO—S cells (5×10⁸ cells/360 ml of media) were split in a 3-L sterile flask using Gibco FreeStyle CHO expression medium+8 mM L-glutamine as culture media. The next day the cells were counted, and total cell number should be approximately 1×10⁹ cells. Two T-75 sterile culture flasks were prepared and were labeled “DNA” and “PEI.” 70 ml of culture media was added to each tube. 2 ml of 1 mg/ml PEI (2 mg) was added to the tube labeled “PEI,” and 1 mg of DNA was added to the tube labeled “DNA” (ratio DNA:PEI=1:2). Both flasks were mixed gently and allowed to stand at room temperature for 15 minutes. The PEI solution was then added to the DNA solution (not the inverse). The tube was then mixed gently and allowed to stand at room temperature for exactly 15 minutes. The DNA/PEI complex (140 ml) was added to the 360 ml of suspension culture in the 3-L flask, and the flasks were incubated on an orbital shaker platform (130 rpm) in an incubator set at 37° C., 8% CO₂. After 4 h of incubation, 500 ml of culture medium was added and incubator temperature was lowered to 31° C. The flask was incubated for 5 days at 31° C., 130 rpm, under 8% CO₂. The cells were then harvested by centrifugation (2000 rpm, 5 min), the conditioned media filtered (0.22 μm) and stored at 4° C.

The purification of the fusion proteins containing a histidine tag was performed with a two-step chromatography including the digestion of the cleavable site by the TEV protease, a highly site-specific cysteine protease that is found in the Tobacco Etch Virus. The purification sequence is as follows. Clarification of the cell culture supernatant was performed by centrifugation or using clarification filters (5-0.6 μm) followed by sterilizing filtration with 0.2 μm cut-off filter. Capture of poly-histidine-tagged proteins was performed using nickel affinity chromatography using the Ni-NTA (Nickel²⁺-nitrilotriacetic acid) Superflow resin (QIAGEN) as follows. First, the column was equilibrated with 50 mM Na₂HPO₄ pH 8.0, 200 mM NaCl, 10% glycerol, 25 mM imidazole. The clarified supernatant was then loaded, followed by a wash using equilibration buffer until UV₂₈₀ absorbance is stable. The proteins were eluted from the column with 50 mM Na₂HPO₄ pH 8.0, 200 mM NaCl, 10% glycerol, 250 mM imidazole. Finally, the column was cleaned in place using 0.5 M NaOH for 30 min contact time, followed by regeneration using equilibration buffer.

Histidine tag removal was performed as follows. The fractions containing a high amount of proteins were dialyzed with TEV protease buffer (50 mM Tris-HCl pH 8.0, 0.5 mM EDTA, and 1 mM DTT). The fusion proteins were then incubated with the TEV protease for 16 h at +4° C. Finally, the fusion protein was dialyzed with Ni-NTA equilibration buffer (50 mM Na₂HPO₄ pH 8.0, 200 mM NaCl, 10% glycerol, 25 mM imidazole).

Capture of poly-histidine tag, TEV-His-tagged, and uncleaved proteins by nickel affinity chromatography using the Ni-NTA Superflow resin (QIAGEN) in Flowthrough mode was performed as follows. First, the column was equilibrated with 50 mM Na₂HPO₄ pH 8.0, 200 mM NaCl, 10% glycerol, 25 mM imidazole. The digested proteins were loaded onto the column, followed by a wash using equilibration buffer until UV₂₈₀ absorbance was stable. The fusion proteins were collected in the flowthrough. The unwanted material was eluted with 50 mM Na₂HPO₄ pH 8.0, 200 mM NaCl, 10% glycerol, 250 mM imidazole. Finally formulation was performed by buffer exchange of the flowthrough fraction containing fusion proteins with PBS buffer.

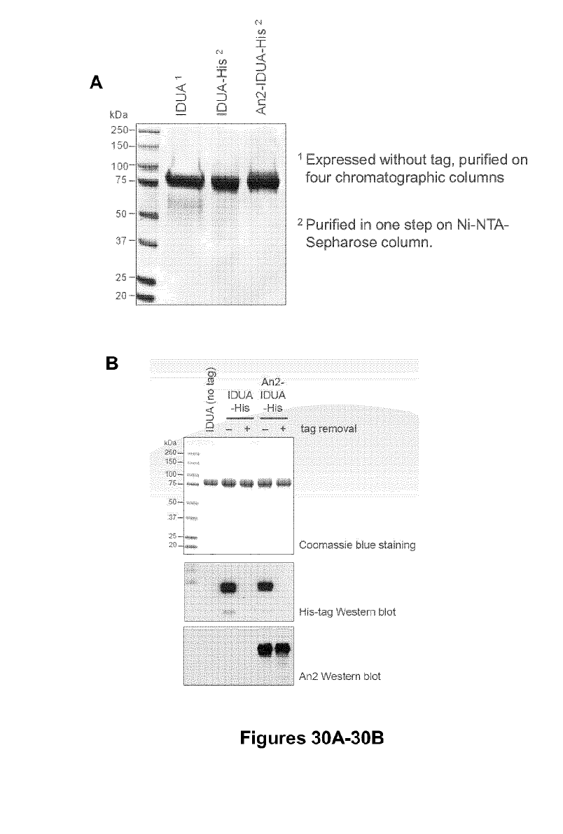

After the first Ni-NTA chromatography step, the His-tag protein eluted show a good purity (FIG. 30A). Furthermore, the His tagged could be removed by TEV cleavage providing purified IDUA or An2-IDUA (FIG. 30B).

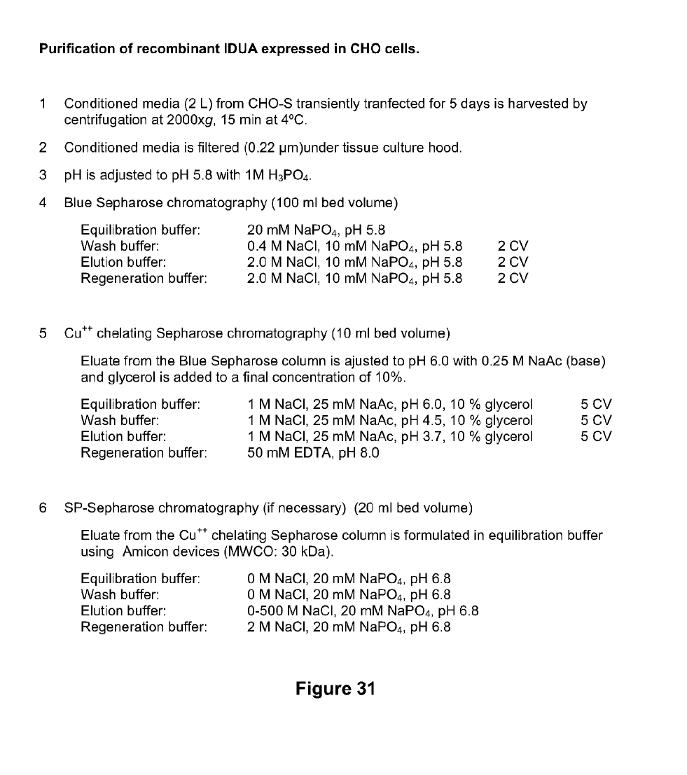

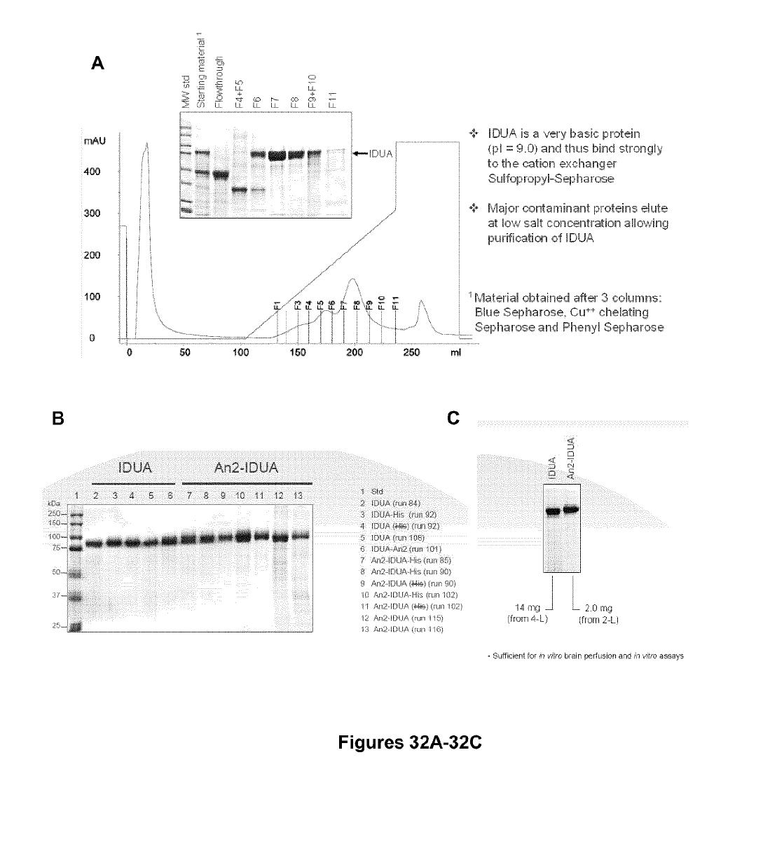

Proteins without histidine were also purified. The use of histidine tag is intended to facilitate protein purification in few steps, but it also requires the removal of the tag by digestion with the TEV protease. All tags, whether large or small, have the potential to interfere with the biological activity of a protein and influence its behavior. In addition, in order to include the TEV digestion site into the constructs, extra amino acids were required, which remain after cleavage on the C-terminal end. This could again influence the protein behavior. Finally, the use of commercially available TEV protease is onerous even at small scale and can contribute up to ˜10% of manufacturing costs. In order to overcome this problem, constructs without a His tag were designed (FIG. 27), and a purification process was developed to achieve high purity. The protocol described in FIG. 31 was used to purify IDUA without a His tag. The purification profile of the IDUA during final step using SP-Sepharose (strong cation-exchange resin) is shown in FIG. 32A. As shown by the SDS-PAGE/Commassie (inset FIG. 32A) of the fractions during elution, high purity could be obtained. Furthermore, FIGS. 32B and 32C show that IDUA and An2-IDUA could be purified reproducibly from multiple batches in amounts sufficient for in vivo brain perfusion and in vitro experiments.

### Example 13

### EPiC-IDUA Activity Testing

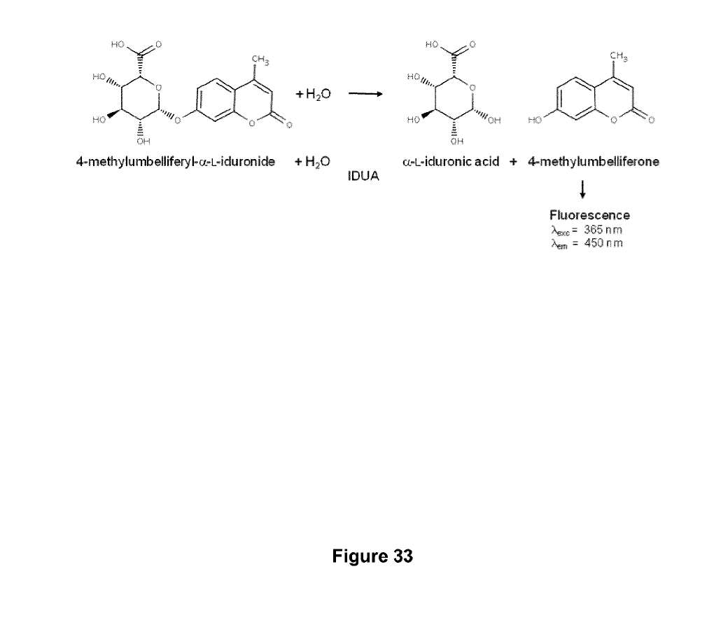

The EPiC-IDUA enzyme activity was determined in vitro by a fluorometric assay with 4-methylumbelliferyl-a-L-iduronide (4-MUBI) as substrate (FIG. 33) using the unpurified proteins (still in culture media). The substrate was hydrolyzed by IDUA to 4-methylumbelliferone (4-MU), which is detected fluorometrically with a Farrand filter fluorometer using an emission wavelength of 450 nm and an excitation wavelength of 365 nM. A standard curve with known amounts of 4-MU was used for determining the concentration of 4-MU in the assay, which is proportional to the IDUA activity.

It is expected that the activity of the enzyme is preserved in the fusion protein and that the fluorometric units should be proportional to the mass of EPiC-IDUA fusion protein added to the substrate.

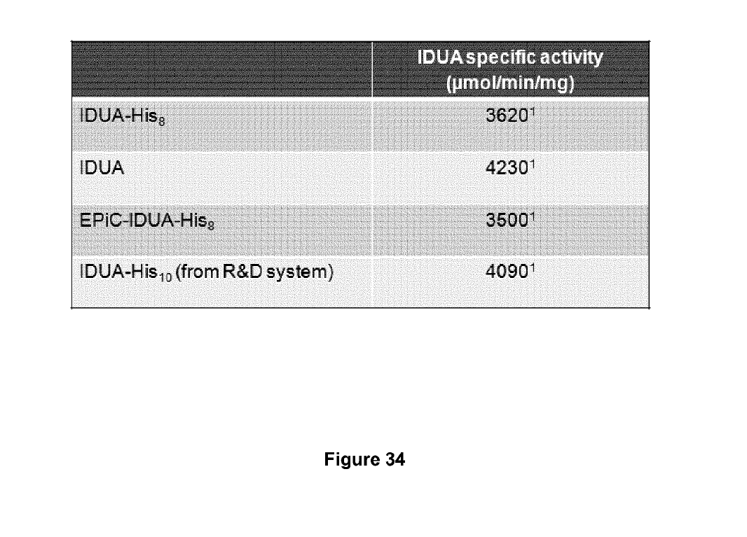

The enzymatic activity of three different proteins expressed in-house in the cell culture supernatant of the cell culture was checked and compared with a commercially available IDUA-10×His. The enzymatic activity of the in-house-produced enzymes showed similar level to the IDUA-10×His (FIG. 34), demonstrating that the enzyme activity is preserved after the fusion with An2.

In order to determine if the expressed proteins were capable of reducing GAG accumulation in cells, fibroblasts taken from an MPS-I patient were used. MPS-I or healthy human fibroblasts (Coriell Institute) were plated in 6-well dishes at 250,000 cells/well in Dulbecco's Modified Eagle Medium (DMEM) with 10% fetal bovine serum (FBS) and grown at 37° C. under 5% CO₂. After 4 days, cells were washed once with phosphate bovine serum (PBS) and once with low sulfate F-12 medium (Invitrogen, catalog #11765-054). One ml of low sulfate F-12 medium containing 10% dialyzed FBS (Sigma, catalog # F0392) and 10 μCi ³⁵S-sodium sulfate was added to the cells, in the absence or presence of recombinant IDUA and EPiC-IDUA proteins. Fibroblasts were incubated at 37° C. under 5% CO₂. After 48 h, medium was removed and cells were washed 5 times with PBS. Cells were then lysed in 0.4 ml/well of 1 N NaOH and heated at 60° C. for 60 min to solubilize proteins. An aliquot is removed for μBCA protein assay. Radioactivity is counted with a liquid scintillation counter. The data is expressed as ³⁵S CPM per μg protein.

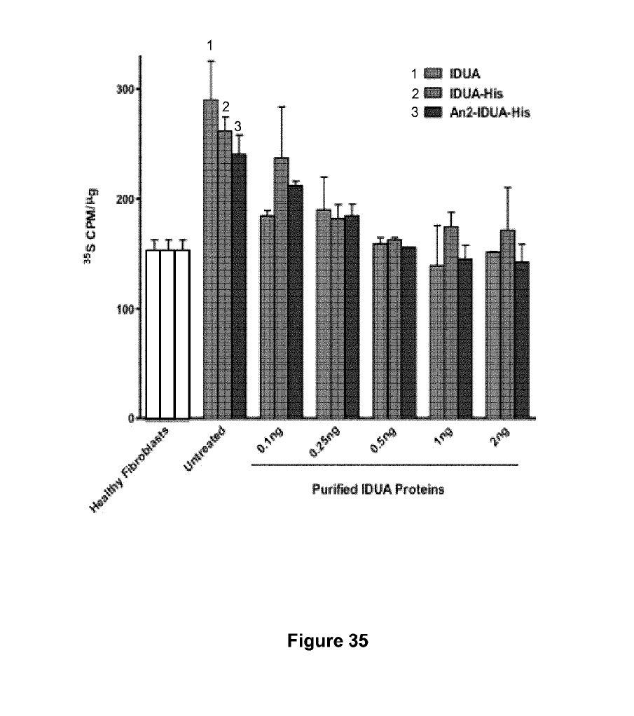

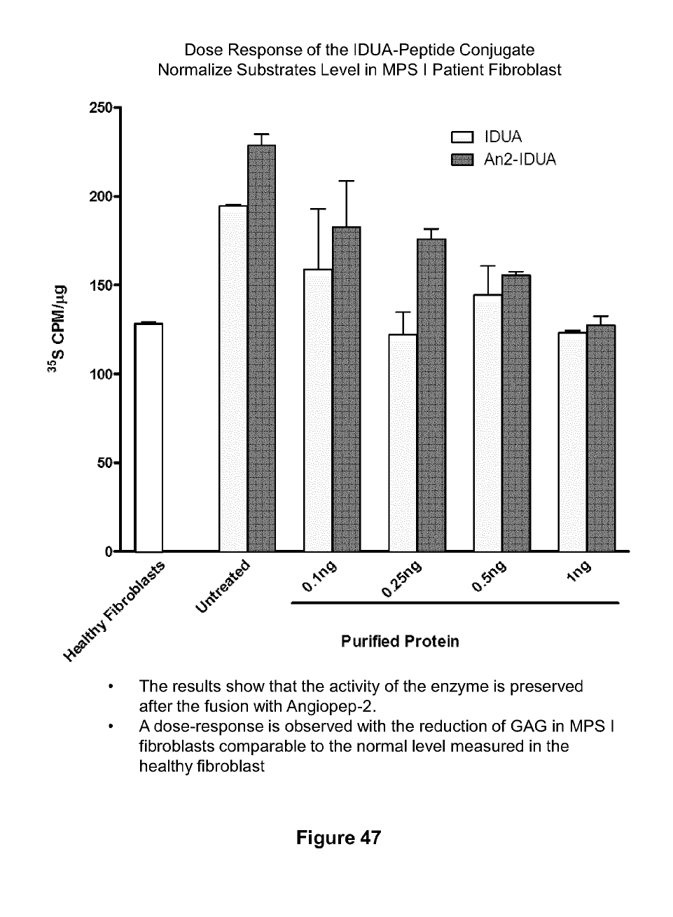

In the first experiment, only IDUA (with and without His tag) and one EPiC-IDUA derivative were tested. The results for the first fusion protein showed that the activity of the enzyme was preserved after the fusion with Angiopep-2. A dose-response was observed with the reduction of GAG in MPS-I fibroblasts comparable to that measured in the healthy fibroblasts (FIG. 35). Similar results were also observed with An2-IDUA as shown in FIG. 47.

### Example 14

In Vitro Evaluation of Intracellular Uptake (Endocytosis) in MPS-I Fibroblasts

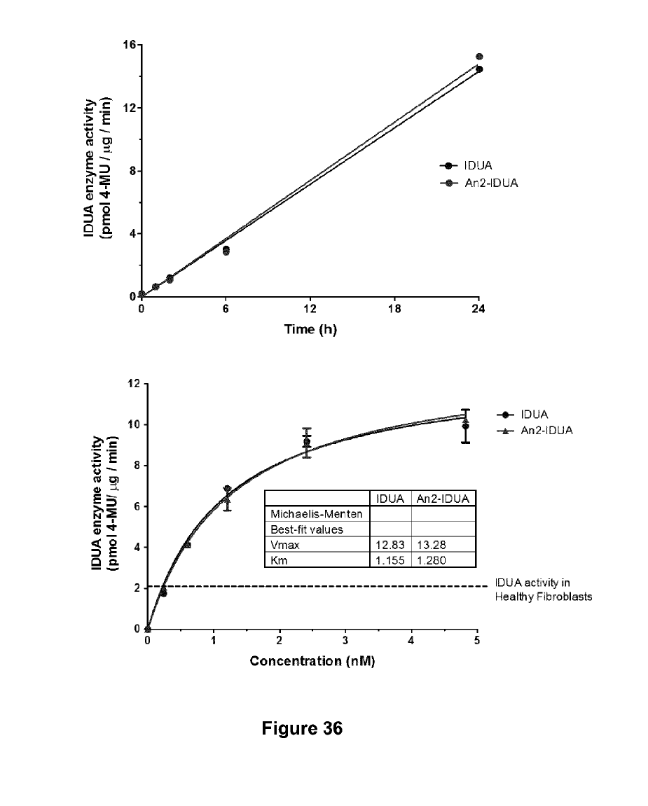

In order to (a) determine if the recombinant IDUA proteins are taken up by cells and (b) compare the level of uptake between native and fusion IDUA, MPS-I fibroblasts were plated in 12-well dishes at 100,000 cells/well in Dulbecco's Modified Eagle Medium (DMEM) with 10% fetal bovine serum (FBS) and grown at 37° C. under 5% CO₂. After 4 days, media was changed and the uptake of IDUA and An2-IDUA fusion protein was evaluated in vitro as follows. Increasing concentration of purified IDUA and An2-IDUA were added to each MPS-I fibroblasts well. Cells were further grown at 37° C. for a maximum of 24 h. The cells were washed thoroughly with PBS to remove the media at different time points within the 24 h exposure interval. The cells were finally lysed in 0.4 M sodium formate, pH 3.5, 0.2% Triton X-100. Enzymatic activity assays were run for each condition. Results are shown in FIG. 36.

Based on these results, An2-IDUA has similar affinity constant for fibroblasts as the native enzyme IDUA, indicating that An2 peptide does not impact the uptake and endocytosis of IDUA. The uptake was found to be time-dependent and linear up to 24 h. In addition, the uptake mechanism appears to be a saturable mechanism with high affinity.

### Example 15

### In Vitro Uptake by MPS-I Fibroblasts in Presence of M6P, an2, and RAP

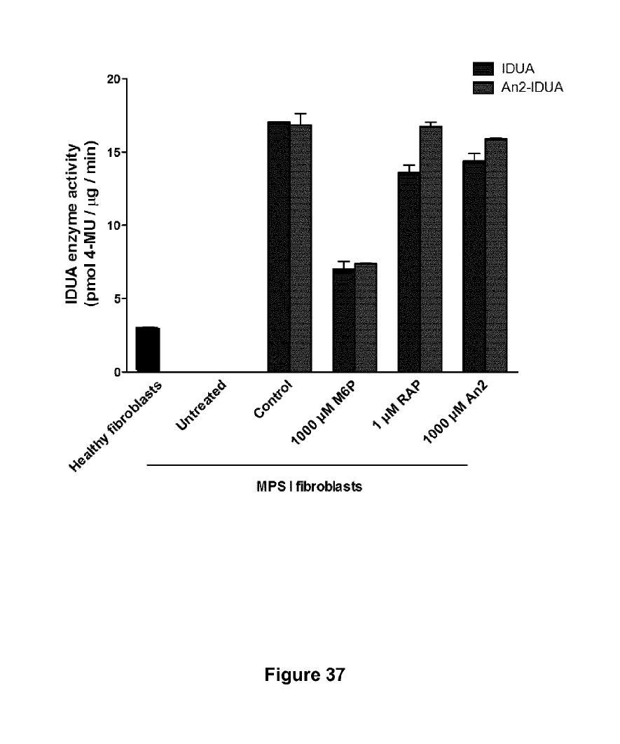

MPS-I fibroblasts cells, as described in previous section, were incubated for 24 h with 2.4 nM of IDUA or An2-IDUA in the presence of an excess of M6P, RAP, or An2. As shown in FIG. 37, the uptake of both An2-IDUA and native IDUA into MPS-I fibroblasts is mainly M6P receptor dependent.

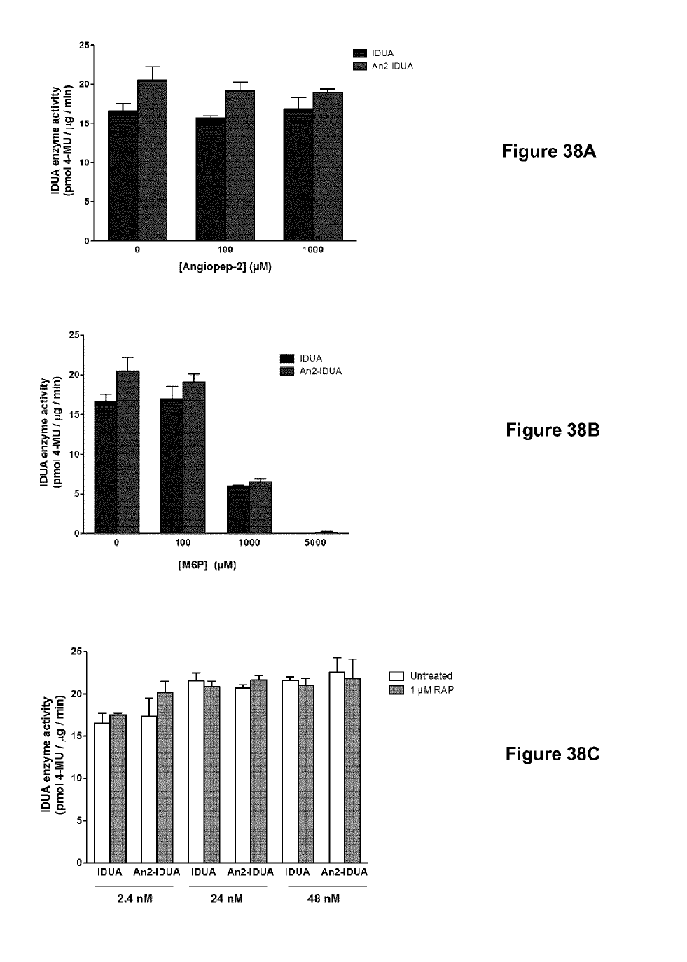

The M6P receptor-dependent uptake of enzyme was further studied with increasing amounts of M6P, An2, and with increasing amount of native and EPIC enzymes in presence of LRP1 inhibitor RAP. The results are shown in FIGS. 38A-38C. These experiments confirmed that, in MPS-I fibroblasts, the uptake of both An2-IDUA and native IDUA was prevented in a dose-dependent manner by co-incubation with free M6P. Additionally, An2 and the LRP1 inhibitor RAP had no effect on An2-IDUA and native IDUA uptake by MPSI fibroblasts, even at high enzyme concentrations.

### Example 16

### In Vitro Uptake by LRP1 High Expressing U87 Glioblastoma Cells

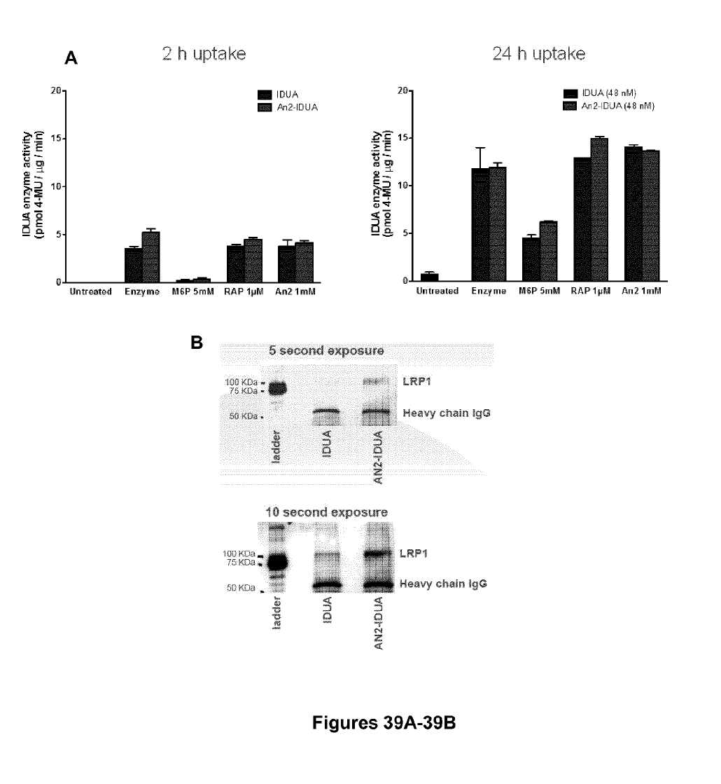

The uptake of IDUA and An2-IDUA was evaluated in U87 glioblastma cells which are known to have high expression of the LRP1 receptor. This experiment was done to further understand the uptake mechanism of IDUA and An2-IDUA by cells and especially to determine if the EPIC compound could play a role in the uptake via LRP1 receptor. The U87 cells were grown and exposed for 2 h and 24 h to IDUA & An2-IDUA in presence of An2 peptide (1 mM), M6P (5 mM) and RAP (1 μm) peptide (LRP1 inhibitor). The results shown in FIG. 39A demonstrate that: 1) the uptake levels of An2-IDUA and native IDUA in U-87 are similar to MPSI fibroblasts; and 2) in U-87, the uptake of both An2-IDUA and native IDUA is mainly M6PR-dependent.

Next LRP1 RAW 264.7 cells expressing cells were incubated with IDUA or An2-IDUA. Immunoprecipitation was performed with an antibody against IDUA followed by western blotting for LRP1. LRP1 was pulled down (FIG. 39B) demonstrating that An2-IDUA interacts with LRP1.

### Example 17

### In Vitro Uptake of Deglycosylated IDUA/an2-IDUA by U87 Glioblastoma Cells

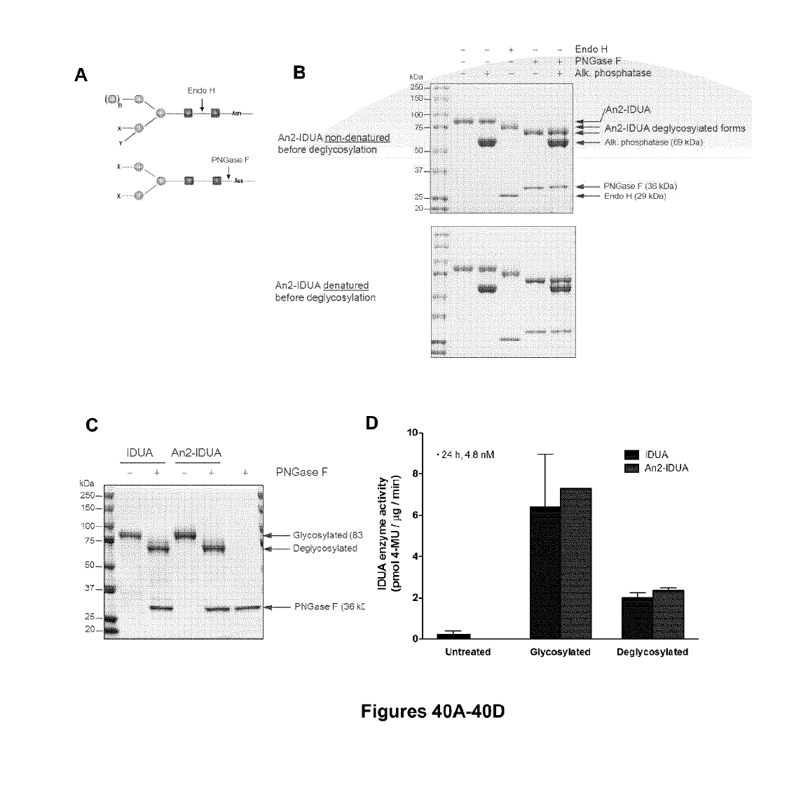

The uptake of IDUA and An2-IDUA was evaluated in U87 glioblastma cells after deglycosylation using PNGase F. This experiment was done to verify the M6P receptor dependant uptake mechanism of IDUA and An2-IDUA by cells. The removal of the glycosylation, including mannose-6-phosphate residues (M6P), was performed by exposing the IDUA/An2-IDUA to N-Glycosidase F, also known as PNGase F, an amidase that cleaves between the innermost GlcNAc and asparagine residues of high mannose (FIG. 40A). An2-IDUA was either denatured or was in the native state prior to deglycosylation (FIG. 40B).

Prior to verifying the enzymatic activity in U87 cells, the enzymes were analyzed by SDS-Page/Coomassie (FIG. 40C). U87 cells were exposed to glycosylated/deglycosylated IDUA/An2-IDUA for 24 h with enzyme concentration of 48 nM. These results (FIG. 40D) show that the glycosylation plays a major role in the uptake mechanism of IDUA/An2-IDUA, confirming all results above which show that the uptake by MPS1 fibroblasts and U87 cells expressing high proportion of LRP1 receptors is mainly mannose 6 phosphate (M6P) receptor dependent. The low level of enzymatic activity measured in U87 cells could be linked to the incomplete deglycosylation of enzymes following PGNase F treatment, as illustrated by the smear of bands between glycosylated/non glycosylated forms in the Coomassie gel above.

### Example 18

### In Vitro Uptake and Localization of an2-IDUA in Lysosomes

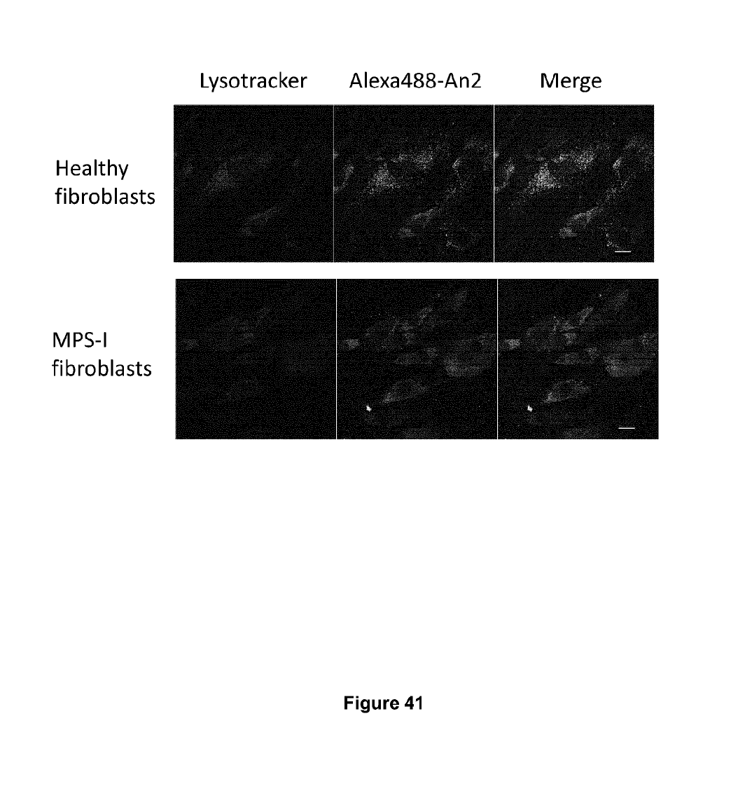

In order to determine whether An2-IDUA fusion proteins reach the lysosomes, co-localization studies were performed using different experimental approaches. To qualify this in vitro method, An2 was labelled with the fluorescent dye Alexa Fluor 488 (a green probe). After the uptake of the fluorescent proteins in fibroblasts from patients with MPS-I, the lysosomes were stained with a lysotracker (a red probe). Confocal microscopy showed good co-localization of the lysotracker and Alexa488-An2 (FIG. 41).

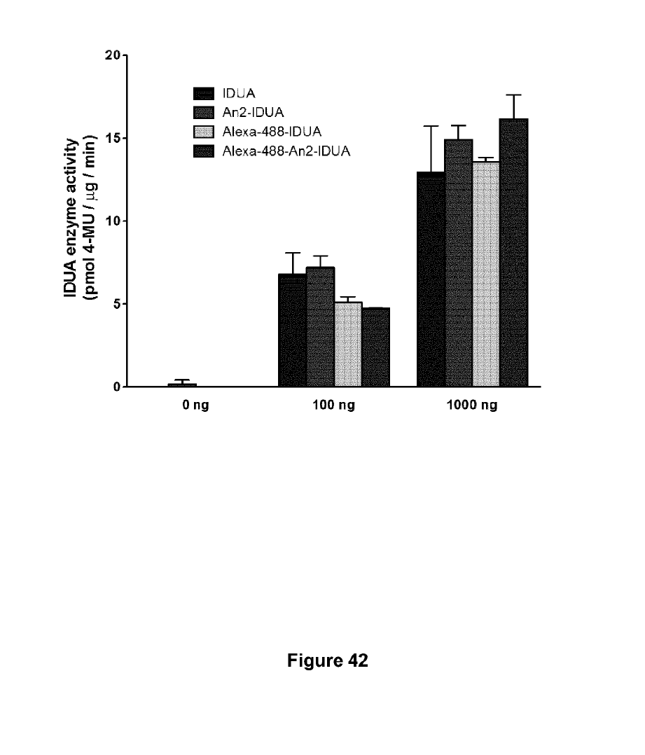

The uptake of IDUA and An2-IDUA was evaluated in U87 glioblastma by comparing the enzymatic activity of non-tagged IDUA/An2-IDUA with green-fluorescent Alexa Fluor 488 tagged material. This experiment was done to verify if the tagging has a detrimental effect on the uptake. The enzymatic activity in U87 cells was evaluated after exposure of the cells to 0, 100, and 1000 ng of tagged/non-tagged enzymes. These results show that tagging IDUA and An2-IDUA with Alexa Fluor488 dye does not impair enzymatic activity and uptake in MPSI fibroblasts (FIG. 42).

### Example 19

### In Vitro Trafficking Studies (Transcytosis)—BBB Transport

In order to measure and characterize the transport of IDUA and EPiC-IDUA derivatives, the purified proteins were radiolabeled with standard procedures using an lodo-beads kit and D-Salt Dextran desalting columns from Pierce (Rockford, Ill., USA). Quantification was done by measuring the amount of radiolabeled molecules crossing the model using trans-well plates. In addition, the integrity of the fusion protein was analyzed by SDS-PAGE or by LS/MS, allowing determination of the molecular weight assuring that no degradation takes place during the transcytosis.

The testing for brain uptake of these fusion proteins was done in mice by an in vivo brain uptake model (aka in situ brain perfusion). This technique allows removal of the blood components and to expose the brain directly to the radiolabeled molecules. Briefly, the uptake of [¹²⁵I]-proteins from the luminal side of mouse brain capillaries was measured using the in situ brain perfusion method adapted in our laboratory for the study of drug uptake in the mouse brain (Cisternino et al., Pharm. Res. 18:183-90, 2001; Dagenais et al., J. Cereb. Blood Flow Metab. 20:381-6, 2000). The brain was perfused for 2-10 min at a flow rate of 1.15 ml/min at 37° C. with radiolabeled compounds. After perfusion of radiolabeled molecules, the brain was further perfused for 60 sec with Krebs buffer to wash away excess [¹²⁵I]-proteins. Mice were then sacrificed to terminate perfusion and the right hemisphere was isolated on ice and capillary depletion immediately performed with ice-cold solutions on Dextran-70 cushion as previously described (Banks et al., J. Pharmacol. Exp. Ther. 302:1062-9, 2002). Aliquots of homogenates, supernatants, pellets, and perfusates were collected to measure their contents and to evaluate the apparent volume of distribution (Vd). The BBB initial transfer constant rate (Kᵢₙ) and regional distribution of radioactive compounds can thus be determined which allows to evaluate the ability of a compound to cross the BBB without interaction of serum proteins. The target rate of uptake of EPiC-IDUA in the brain parenchyma (Kᵢₙ) should be at a minimum of 10⁻⁴ ml/g/sec. As a comparison, the reported Kᵢₙ for glucose is 9.5×10⁻³ (Mandula et al., J. Pharmacol. Exp. Ther. 317:667-75, 2006), the Kᵢₙ for alcohol is 1.8×10⁻⁴ (Gratton et al., J. Pharm. Pharmacol. 49:1211-6, 1997) and the Kᵢₙ for morphine is 1.6×10⁻⁴ (Seelbach et al., J. Neurochem. 102:1677-90, 2007).

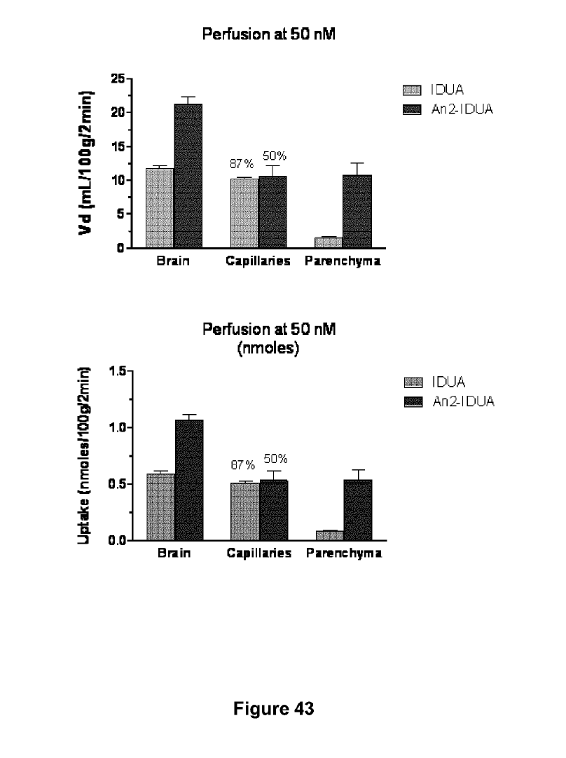

The BBB transport evaluation was performed for IDUA and EPIC-IDUA with the following parameters: radiolabelled material concentration of 50 nM, perfusion time of 2 min at 1.15 ml/min at 37° C., and rinse time of 30 s. The results (FIG. 43) indicate that IDUA alone may bind or may be trapped in brain capillaries and that low amount reaches the brain parenchyma. One explanation could be the fact that IDUA has an isoelectric point around 9. Thus, the protein is positively charged at neutral pH. In the case of An2-IDUA, we observed an increased in the distribution volume in the total brain. Interestingly, higher amount is found in the brain parenchyma (about 7-fold) compared to the native enzyme. Overall, these results indicate that the addition of An2 increases the transport of IDUA across the BBB.

### Example 20

### In Vitro BBB Evaluation Using BBB Model (CELLIAL Technologies)

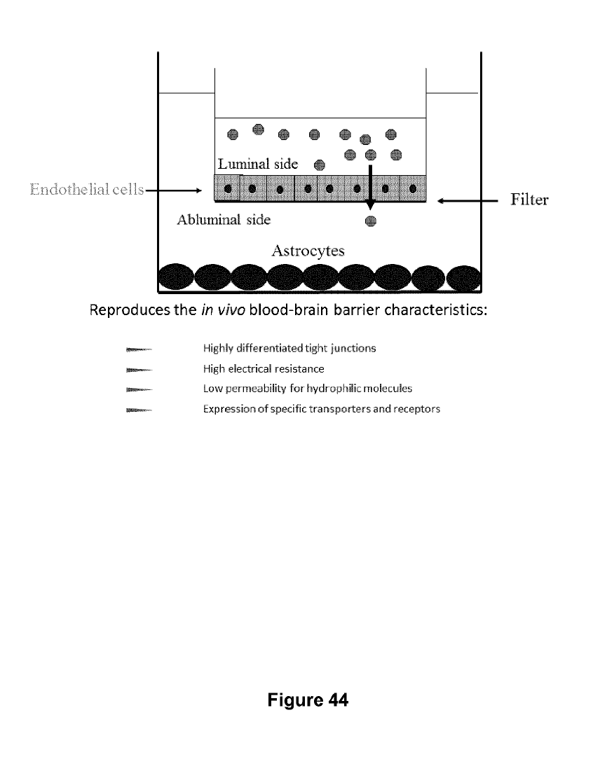

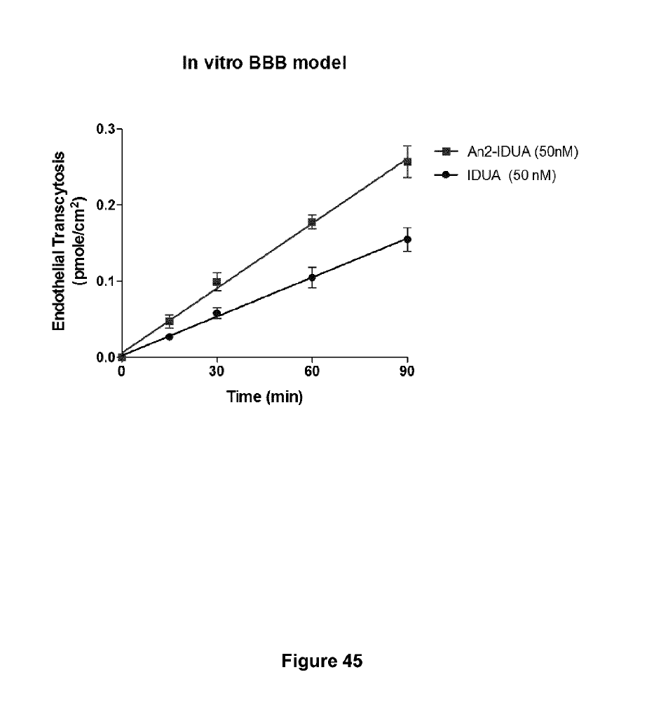

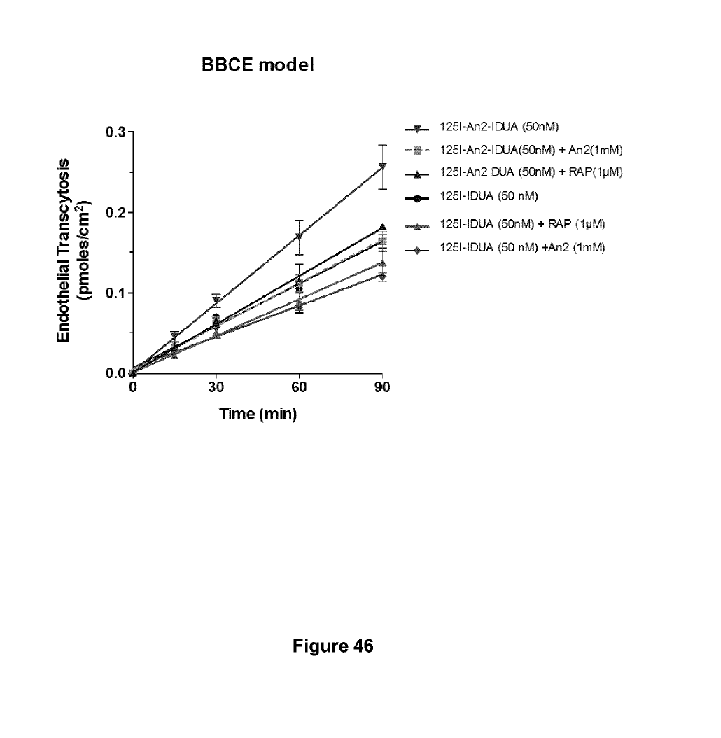

The transport of the EPiC-Enzyme derivatives across the BBB was also evaluated using an in vitro BBB model composed of a co-culture of bovine brain capillary endothelial cells with newborn rat astrocytes (FIG. 44). In order to measure and characterize the transport of IDUA and An2-IDUA derivatives, the purified proteins were radiolabeled with standard procedures. Quantification was done by measuring the amount of radiolabeled molecules crossing the model using trans-well plates. In addition, the integrity of the fusion protein was analyzed by SDS-PAGE or by LS/MS allowing determination of the molecular weight, assuring that no degradation took place during transcytosis. The transport of An2-IDUA and IDUA enzyme was compared using the in vitro BBB protocol. The results, shown in FIG. 45, indicate that the transport across the BBB of EPIC-IDUA was increased ˜2 fold compared to the enzyme only.

The transport of EPIC-IDUA and IDUA through the BBB endothelial cells was also evaluated in presence of LRP1 receptor competitors like RAP and Ant. The results, presented in FIG. 46, demonstrate that the passage of IDUA through the BBB endothelial cell is An2-transport dependent.

### Example 21

### Enzymatic Activity of an2-IDUA in MPS-I Knock Out Mice

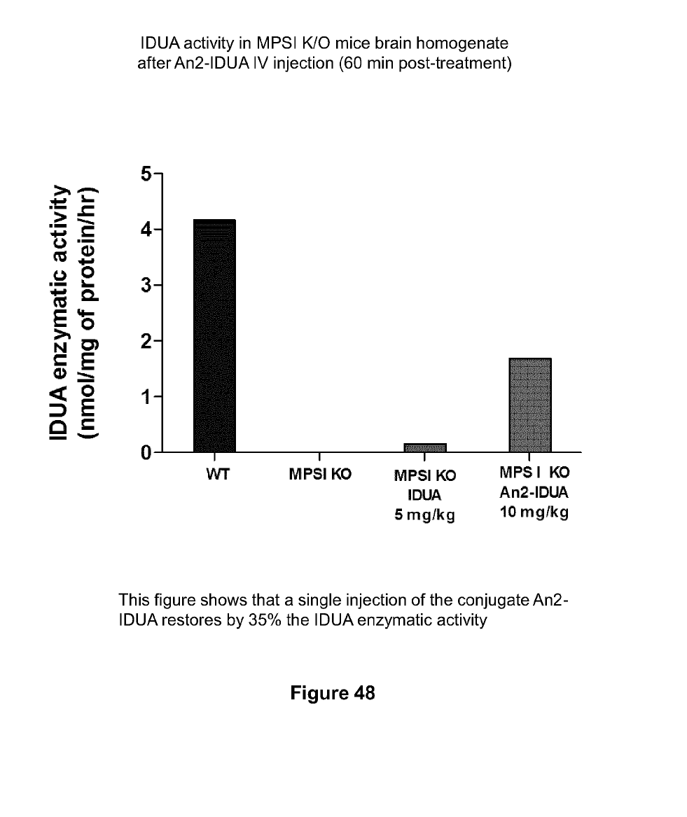

IDUA activity was measured in homogenates of mice brains prepared from MPS-I knock out mice, one hour after intravenous injection of An2-IDUA. FIG. 48 shows that a single injection of An2-IDUA restores by 35% the IDUA enzymatic activity in MPS-I knock out mice brain homogenate.

### Example 22

### Chemical Conjugation of IDUA to a Peptide

The peptide targeting moiety, such as Angiopep-2, may be attached to IDUA by a chemical linker. In one example, this is achieved using an SATA linker, which is described above. Chemical conjugation may be achieved using the following scheme.

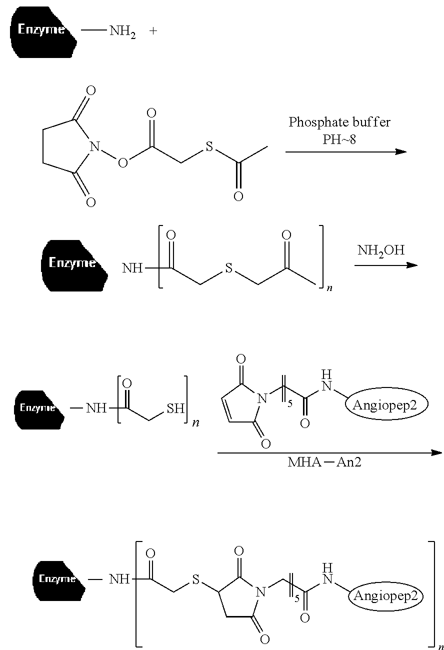

In this scheme, four equivalents of SATA are reacted with the enzyme in phosphate buffer at pH 8, thus conjugating the linker to the enzyme. The enzyme-linker is then deprotected with hydroxylamine to obtain free sulphydryl intermediate of IDUA. This compound was then conjugated to six equivalents of MHA-Angiopep-2, to generate the enzyme-peptide conjugate.

In another example, the enzyme is reacted with Traut's reagent (2-iminothialone), which is then conjugated to six equivalents of MHA-Angiopep-2, as shown below.

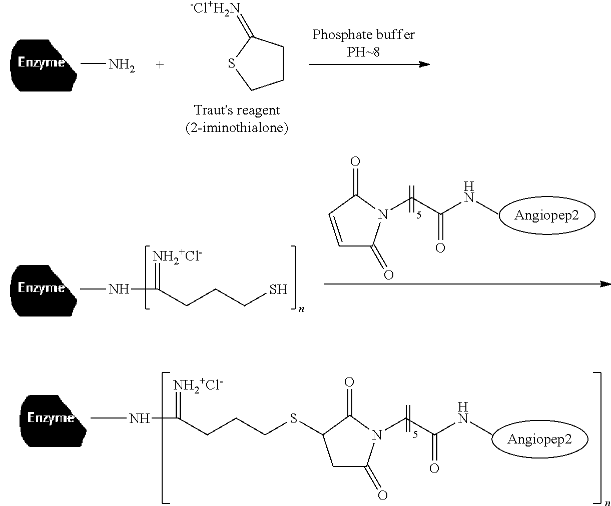

### Other Embodiments

All patents, patent applications, and publications mentioned in this specification are herein incorporated by reference including U.S. Provisional Application No. 61/565,764, filed Dec. 1, 2011 and U.S. Provisional Application No. 61/660,564, filed Jun. 15, 2012, to the same extent as if each independent patent, patent application, or publication was specifically and individually indicated to be incorporated by reference.

What is claimed is:

1. A compound comprising (a) a peptide or peptidomimetic targeting moiety less than 50 amino acids, wherein said targeting moiety comprises an amino acid sequence that is at least 70% identical to any of SEQ ID NOS:1-105 and 107-117 and (b) a lysosomal enzyme, an active fragment thereof, or an analog thereof, wherein said targeting moiety and said enzyme are joined by a linker.

2-32. (canceled)

33. The compound of claim 1, wherein said compound further comprises a second targeting moiety, said second targeting moiety being joined to said compound by a second linker.

34. The compound of claim 1, wherein said targeting moiety is capable of transporting said enzyme to the lysosome and/or across the blood brain barrier.

35. The compound of claim 1, wherein said compound maintains lysosomal enzymatic activity in an enzymatic assay and/or in a cellular assay.

36. The compound claim 1, wherein said targeting moiety comprises the sequence of Angiopep-2 (SEQ ID NO:97).

37. The compound of claim 1, wherein the peptidomimetic targeting moiety contains one or more D-amino acids.

38. The compound of claim 37, wherein said targeting moiety comprises one or more D-isomers of the amino acid recited in SEQ ID NO: 97.

39-41. (canceled)

42. The compound of claim 40, wherein said targeting moiety comprises four or more D-isomers of the amino acid sequence recited in SEQ ID NO: 97.

43. The compound of claim 42, wherein said targeting moiety has the formula Thr-Phe-Phe-Tyr-Gly-Gly-Ser-D-Arg-Gly-D-Lys-D-Arg-Asn-Asn-Phe-D-Lys-Thr-Glu-Glu-Tyr.

44. The compound of claim 1, wherein the peptidomimetic targeting moiety contains N-acyl derivatives of the amino terminal or of another free amino group.

45. The compound of claim 44, wherein the peptidomimetic targeting moiety contains an acetyl group.

46. The compound of claim 1, wherein said linker is a covalent bond or one or more amino acids.

47. The compound of claim 46, wherein said covalent bond is a peptide bond.

48. The compound of claim 1, wherein said compound is a chemical conjugate.

49. The compound of claim 48, wherein said linker is conjugated to said enzyme through a free amine on said enzyme.

50. The compound of claim 48, wherein said linker is conjugated to said targeting moiety through a free amine on said targeting moiety.

51. The compound of claim 48, wherein said compound has the structure: 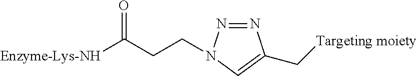
wherein the “Lys-NH” group represents either a lysine present in the enzyme or an N-terminal or C-terminal lysine.

52. (canceled)

53. The compound of claim 48, wherein said compound has the structure: 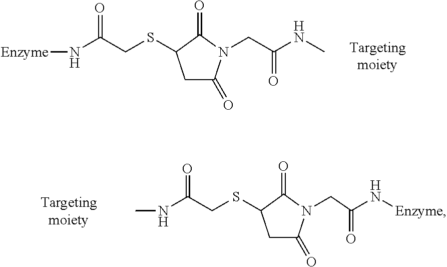
wherein each —NH— group represents a primary amino present on the targeting moiety and the enzyme, respectively.

54-56. (canceled)

57. A pharmaceutical composition comprising a compound of claim 1 and a pharmaceutically acceptable carrier.

58. A method of treating or treating prophylactically a subject having a lysosomal storage disorder, said method comprising administering to said subject a compound of claim 1.

59. The method of claim 58, wherein said subject has neurological symptoms.

60-62. (canceled)

63. The method of claim 58, wherein said administering comprises parenteral administration.
